(19) United States
(12) Patent Application Publication
(10) Pub. No.: US 2023/0159644 A1
(43) Pub. Date: May 25, 2023

(54) FLT3-TARGETED CHIMERIC ANTIGEN RECEPTOR MODIFIED CELLS FOR TREATMENT OF FLT3-POSITIVE MALIGNANCIES

(71) Applicant: City of Hope, Duarte, CA (US)

(72) Inventors: Michael Caligiuri, Wilmington, DE (US); Jianhua Yu, Wilmington, DE (US)

(21) Appl. No.: 17/919,178

(22) PCT Filed: Apr. 16, 2021
(86) PCT No.: PCT/US2021/027821

§ 371 Date: Oct. 14, 2022

Related U.S. Application Data
(60) Provisional application No. 63/011,819, filed on Apr. 17, 2020.

(51) Int. Cl. C07K 16/28; A61P 35/02

(52) U.S. Cl. CPC C07K 16/2863; A61P 35/02; A61K 2039/5156

(57) ABSTRACT

Provided are compositions comprising a population of autologous or allogeneic cells transduced by a nucleic acid molecule encoding a chimeric antigen receptor (CAR) specifically recognizes and binds FMS-like tyrosine kinase 3 (FLT3), methods of formulating preparing such CAR expressing cells and methods of use as anti-cancer agents.

### CROSS-REFERENCE TO RELATED APPLICATION

This application claims priority under 35 U.S.C. § 119(e) of U.S. Provisional Application No. 63/011,819, filed Apr. 17, 2020, the contents of which is incorporated by reference in its entirety into the present application.

### TECHNICAL FIELD

This disclosure concerns chimeric antigen receptors (CAR) engineered to bind to FMS-like tyrosine kinase 3 (FLT3) expressing cells, T cells or NK cells expressing such CAR, methods of formulating such CAR T or NK cells and methods of use as anti-cancer agents.

### BACKGROUND

FMS-like tyrosine kinase 3 (FLT3) is a transmembrane protein expressed on normal hematopoietic stem and progenitor cells. It is also expressed on malignant blasts in acute myeloid leukemia (AML).

Cells expressing a FLT3-specific chimeric antigen receptor (CAR) can suppress in vivo growth of solid and blood cancers such as leukemia, and prolong the survival of leukemia-bearing mice. (Chen et al., Leukemia 31:1830, 2017). Thus, remains needed is a FLT3-specific CAR therapy that efficiently treats a cancer in a patient.

### SUMMARY OF THE DISCLOSURE

For patients with FLT3 expression on the surface of cancer cells, such as leukemic blasts, FLT3-CAR T or NK cells could be considered for treatment of refractory disease or up front treatment, for example in the elderly with AML, given their dismal prognosis with standard therapy.

Described herein are nucleic acid molecules encoding a FLT3-specific CAR (also term “FLT3 CAR”). The nucleic acid molecules include an improved coding sequence (e.g., SEQ ID NO: 1) for a FLT3-specific CAR that allows higher expression of the CAR and greater effectiveness than a previously designed coding sequence.

In one aspect, the FLT3 CAR nucleic acid molecule comprises, or consists essentially of, or yet further consists of:

(SEQ ID NO: 1)
ATGGGGTGGTCAAGCATTATTCTGTTTCTGGTCGC
TACCGCTACAGGCGTCCATCAGGTCCAGCTGCAGC
AGCCCGGAGCCGAACTGGTGAAGCCCGGCGCCTCC
CTGAAGCTGTCTTGCAAGAGCAGCGGCTACACATT
CACCTCCTATTGGATGCACTGGGTGCGGCAGCGGC
CCGGCCACGGCCTGGAGTGGATCGGCGAGATCGAC
CCCTCTGATAGCTACAAGGACTATAACCAGAAGTT
TAAGGATAAGGCCACACTGACCGTGGACCGGTCTA
GCAATACAGCCTACATGCACCTGTCCTCTCTGACC
TCCGACGATTCTGCCGTGTACTATTGCGCCAGGGC
CATCACCACAACCCCTTTCGATTTTTGGGGCCAGG
GCACAACCCTGACCGTGAGCAGCGGAGGAGGAGGC
AGCGGAGGAGGAGGCTCCGGCGGCGGCGGCTCTGA
CATCGTGCTGACCCAGTCCCCAGCCACACTGAGCG
TGACCCCTGGCGACTCCGTGTCTCTGAGCTGTCGG
GCCTCCCAGTCTATCAGCAACAATCTGCACTGGTA
TCAGCAGAAGAGCCACGAGTCCCCTAGGCTGCTGA
TCAAGTATGCCTCCCAATCTATCAGCGGCATCCCA
AGCCGCTTCTCCGGCTCTGGCAGCGGCACAGACTT
CACCCTGTCTATCAACAGCGTGGAGACAGAGGACT
TCGGCGTGTATTTTTGTCAGCAGTCTAATACATGG
CCATATACATTCGGAGGAGGAACTAAACTGGAAAT
CAAACGACTCGAGCCCAAATCTTGTGACAAAACTC
ACACATGCCCACCGTGCCCGGATCCCAAAGGTACC
TTTTGGGTGCTGGTGGTGGTTGGTGGAGTCCTGGC
TTGCTATAGCTTGCTAGTAACAGTGGCCTTTATTA
TTTTCTGGGTGAGGAGTAAGAGGAGCAGGCTCCTG
CACAGTGACTACATGAACATGACTCCCCGCCGCCC
CGGGCCCACCCGCAAGCATTACCAGCCCTATGCCC
CACCACGCGACTTCGCAGCCTATCGCTCCAGAGTG
AAGTTCAGCAGGAGCGCAGACGCCCCCGCGTACCA
GCAGGGCCAGAACCAGCTCTATAACGAGCTCAATC
TAGGACGAAGAGAGGAGTACGATGTTTTGGACAAG
AGACGTGGCCGGGACCCTGAGATGGGGGGAAAGCC
GAGAAGGAAGAACCCTCAGGAAGGCCTGTACAATG
AACTGCAGAAAGATAAGATGGCGGAGGCCTACAGT
GAGATTGGGATGAAAGGCGAGCGCCGGAGGGGCAA
GGGGCACGATGGCCTTTACCAGGGTCTCAGTACAG
CCACCAAGGACACCTACGACGCCCTTCACATGCAG
GCCCTGCCCCCTCGC.

SEQ ID NO:1 encodes a polypeptide having the sequence:

(SEQ ID NO: 2)
MGWSSIILFLVATATGVHQVQLQQPGAELVKPGAS
LKLSCKSSGYTFTSYWMHWVRQRPGHGLEWIGEID
PSDSYKDYNQKFKDKATLTVDRSSNTAYMHLSSLT
SDDSAVYYCARAITTTPFDFWGQGTTLTVSSGGGG
SGGGGSGGGGSDIVLTQSPATLSVTPGDSVSLSCR
ASQSISNNLHWYQQKSHESPRLLIKYASQSISGIP
SRFSGSGSGTDFTLSINSVETEDFGVYFCQQSNTW
PYTFGGGTKLEIKRLEPKSCDKTHTCPPCPDPKGT
FWVLVVVGGVLACYSLLVTVAFIIFWVRSKRSRLL
HSDYMNMTPRRPGPTRKHYQPYAPPRDFAAYRSRV
KFSRSADAPAYQQGQNQLYNELNLGRREEYDVLDK
RRGRDPEMGGKPRRKNPQEGLYNELQKDKMAEAYS
EIGMKGERRRGKGHDGLYQGLSTATKDTYDALHMQ
ALPPR

In one aspect, the CAR includes, comprises, or consists essentially of, or yet further consists of: a signal sequence (e.g., MGWSSIILFLVATATGVH; SEQ ID NO:3); a FLT3-targeted single chain variable fragment (scFv) (QVQLQQPGAELVKPGASLKLSCKSSGYTFTSYWMHWVRQRPGHGLEWIGEIDPSDS YKDYNQKFKDKATLTVDRSSNTAYMHLSSLTSDDSAVYYCARAITTTPFDFWGQGT TLTVSSGGGGSGGGGSGGGGSDIVLTQSPATLSVTPGDSVSLSCRASQSISNNLHWY QQKSHESPRLLIKYASQSISGIPSRFSGSGSGTDFTLSINSVETEDFGVYFCQQSNTWPY TFGGGTKLEIKR; SEQ ID NO: 4), a spacer (i.e., a hinge domain, LEPKSCDKTHTCPPCPDPKGT; SEQ ID NO: 5); a CD28 transmembrane domain (FWVLVVVGGVLACYSLLVTVAFIIFWV; SEQ ID NO:6), a CD28 co-stimulatory domain (RSKRSRLLHSDYMNMTPRRPGPTRKHYQPYAPPRDFAAYRS; SEQ ID NO:7); and a CD3zeta intracellular signaling domain (RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR; SEQ ID NO:8). In some embodiments, the scFv comprises, or consists essentially of, or yet further consists of a heavy chain variable region: QVQLQQPGAELVKPGASLKLSCKSSGYTFTSYWMHWVRQRPGHGLEWIGEIDPSDS YKDYNQKFKDKATLTVDRSSNTAYMHLSSLTSDDSAVYYCARAITTTPFDFWGQGT TLTVSS; or amino acid (aa) 1 to aa 118 of SEQ ID NO: 4) and a light chain variable region (DIVLTQSPATLSVTPGDSVSLSCRASQSISNNLHWYQQKSHESPRLLIKYASQSISGIP SRFSGSGSGTDFTLSINSVETEDFGVYFCQQSNTWPYTFGGGTKLEIKR; or aa 134 to aa 241 of SEQ ID NO: 4) linked by a peptide linker (e.g., GGGGSGGGGSGGGGS; or aa 119 to aa 133 of SEQ ID NO: 4).

In some cases, the CAR can be produced using a vector in which the CAR open reading frame is followed by a T2A ribosome skip sequence and a truncated EGFR (referred to herein as EGFRt or tEGFR) or truncated CD19 (referred to herein as CD19t or tCD19). In this arrangement, co-expression of EGFRt or CD19t provides an inert, non-immunogenic surface marker that allows for accurate measurement of gene modified cells, and enables positive selection of gene-modified cells, as well as efficient cell tracking of the therapeutic T or NK cells in vivo following adoptive transfer. Efficiently controlling proliferation to avoid cytokine storm and off-target toxicity is an important hurdle for the success of T cell immunotherapy. The EGFRt or CD19t incorporated in a vector such as a lentiviral or retroviral vector can act as suicide gene to ablate the CAR+ T or NK cells in cases of treatment-related toxicity.

In one aspect, provided herein is a nucleic acid molecule encoding an anti-FMS-like tyrosine kinase 3 (FLT3) chimeric antigen receptor (CAR). The nucleic acid molecule comprises, or alternatively consists essentially of, or yet further consists of the polynucleotide sequence of nucleotide (nt) 55 to nt 777 of SEQ ID NO:1 or an equivalent thereof that encodes the FLT3 scFv antigen binding fragment. In some embodiments, the equivalent is at least about 75% (including but not limited to at least about 90%, or at least about 95%, or at least about 99%) identical to nucleotide (nt) 55 to nt 777 of SEQ ID NO: 1 (that encodes the FLT3 scFv antigen binding fragment) while maintaining at least one of the optimized nucleotide changes. Additionally or alternatively, the equivalent comprises, or alternatively consists essentially of, or yet further consists of the polynucleotide of nucleotide (nt) 55 to nt 777 of SEQ ID NO: 12 or an equivalent thereof that encodes the FLT3 scFv antigen binding fragment. In further embodiments, the equivalent does not comprise, or consist essentially of, or consist of (nt) 55 to nt 777 of SEQ ID NO: 14. In some embodiments, the nucleic acid molecule further comprises, or consists essentially of, or yet further consists of a polynucleotide encoding a truncated CD19 or a truncated EGFR.

In some embodiments, the nucleic acid molecule comprises, or alternatively consists essentially of, or yet further consists of the polynucleotide sequence of nucleotide (nt) 1 to nt 777 of SEQ ID NO:1 or an equivalent thereof that encodes a signal peptide and the FLT3 scFv antigen binding region. In some embodiments, the equivalent is at least about 75% (including but not limited to at least about 90%, or at least about 95%, or at least about 99%) identical to nucleotide (nt) 1 to nt 777 of SEQ ID NO: 1 while maintaining the optimized nucleotide changes. Additionally or alternatively, the equivalent comprises, or alternatively consists essentially of, or yet further consists of the polynucleotide of nucleotide (nt) 1 to nt 777 of SEQ ID NO: 12. In further embodiments, the equivalent does not comprise, or consist essentially of, or consist of (nt) 1 to nt 777 of SEQ ID NO: 14.

In some embodiments, the nucleic acid molecule comprises, or alternatively consists essentially of, or yet further consists of the polynucleotide sequence of SEQ ID NO:1 or an equivalent thereof. In some embodiments, the equivalent is at least about 75% (including but not limited to at least about 90%, or at least about 95%, or at least about 99%) identical to SEQ ID NO: 1 while maintaining the optimized nucleotide changes. Additionally or alternatively, the equivalent comprises, or alternatively consists essentially of, or yet further consists of the polynucleotide of SEQ ID NO: 12.

Also provided is a vector comprising, or alternatively consisting essentially of, or yet further consisting of a nucleic acid molecule encoding the FLT3 CAR as disclosed herein or a complementary nucleic acid molecule thereof. In some embodiments, the vector is a plasmid vector, a viral vector, an expression vector. In further embodiments, the vector is a retroviral vector. In yet further embodiments, the vector is a lentiviral vector.

In a further aspect, provided is a population of human T or NK cells comprising a nucleic acid molecule as disclosed herein encoding the FLT3 CAR or a vector as disclosed herein encoding the FLT3 CAR. Additionally provided is an isolated cell comprising a nucleic acid molecule as disclosed herein encoding the FLT3 CAR or a vector as disclosed herein encoding the FLT3 CAR, or a population thereof. In some embodiments, the cell is selected from the group of: an immune cell, an NK cell, a T cell, a stem cell, a progenitor cell or a precursor cell to the immune cell, the NK cell or the T cell.

In yet a further aspect, provided is a composition comprising, or alternatively consisting essentially of, or yet further consisting of a population of cells containing or expressing the FLT3 CAR as disclosed herein, or an isolated cell expressing the FLT3 CAR as disclosed herein, and a carrier, and optionally a stabilizer, preservative or cryopreservative.

In one aspect, provided is a method of treating a human patient suffering from cancer such as acute myeloid leukemia. The method comprises, or alternatively consists essentially of, or yet further consists of administering a population of autologous or allogeneic human T or NK cells comprising a nucleic acid molecule encoding the FLT3 CAR as disclosed herein or a vector comprising the nucleic acid molecule encoding the FLT3 CAR as disclosed herein. One of skill in the art can determine when the patient has been treated using clinical and subclinical parameters known in the art, such as a reduction in tumor burden, inhibition of metastasis, enhanced overall survival, reduced toxicity, prolonged progression free survival, slowing or halting of cancer, reduction in tumor markers or other clinical symptoms as compared to treatment with other known therapies or in the absence of therapy. These methods can be combined with other known anti-tumor or cancer therapies as a second line, third line, fourth line, or fifth line therapy, or be provided as a first line therapy.

In another aspect, provided is a method of treating a patient in need thereof. The method comprises, or alternatively consists essentially of, or yet further consists of administering a FLT3 CAR cell population as disclosed herein to the patient.

In yet another aspect, provided is a method of preparing CAR T or NK cells. The method comprises, or alternatively consists essentially of, or yet further consists of providing a population of autologous or allogeneic human T or NK cells and transducing the T or NK cells with a vector as disclosed herein or a nucleic acid molecule as disclosed herein.

Additionally provided is a method of preparing a FLT3-CAR expressing cell. The method comprises, or alternatively consists essentially of, or yet further consists of transducing the cell with a vector as disclosed herein or a nucleic acid molecule as disclosed herein.

Compositions and kits for use in a method as disclosed herein is also provided.

### BRIEF DESCRIPTION OF THE DRAWINGS

FIG. 1 provides a schematic diagram showing how pHIV-7 was constructed.

FIG. 2 provides an illustration of pHIV-7.

FIG. 3 provides an illustration of a plasmid for producing a retroviral vector genome expressing the FLT3-specific CAR.

FIGS. 4A-4D provide flow cytometry results evaluating CAR expression on four T cell groups, i.e., T cells expressing GFP serving as a control (FIG. 4A), T cell expressing tCD19 also serving as a control (FIG. 4B), T cells expressing tCD19 and FLT3-specific CAR encoded by the nucleic acid molecule without optimization (FIG. 4C), and T cells expressing tCD19 and FLT3-specific CAR encoded by the optimized nucleic acid molecule (FIG. 4D).

FIGS. 5A-5B plot tumor lysis percentages tested at two different effector to target cell ratios (E:T) using two different target cancer cells, i.e., MOLM13 and U937. FIG. 5A shows results tested at an E:T of 1:5, while FIG. 5B shows results tested at an E:T of 1:25. For each type of the target cells and each E:T, four groups of T cells were tested, i.e., T cells expressing GFP (GFP), T cell expressing tCD19 (tCD19), T cells expressing tCD19 and FLT3-specific CAR encoded by the nucleic acid molecule without optimization (Before Opt), and T cells expressing tCD19 and FLT3-specific CAR encoded by the optimized nucleic acid molecule (After Opt); and the tumor lysis percentages were plotted as bars in a set of four following the same order.

FIGS. 6A-6C provide concentrations of interleukin 2 (IL-2) in the supernatant of co-culture of cancer cells and T cells isolated from Donor 1 to Donor 3, respectively. The T cells were engineered to express GFP (GFP control), or tCD19 (tCD19 control), or tCD19 and FLT3-specific CAR encoded by the nucleic acid molecule without optimization (Before Opt. FLT3/CD19), or tCD19 and FLT3-specific CAR encoded by the optimized nucleic acid molecule (After Opt. FLT3/CD19). And then each group of the engineered T cells was incubated with two cancer cell lines separately: MOLM13 cells are FLT3 expressing (i.e., FLT3+) tumor target while U937 cells do not express FLT3 and served as a negative control. Accordingly, two bars are plotted in FIGS. 6A-6C for each Donor and each T cell group: the left one indicates IL-2 concentration in the co-culture with MOLM13, while the right one indicates IL-2 concentration in the co-culture with U937.

FIGS. 7A-7C provide concentrations of Interferon gamma (i.e., IFNγ or IFN-gamma) in the supernatant of co-culture of cancer cells and T cells isolated from Donor 1 to Donor 3, respectively. The T cells were engineered to express GFP (GFP control), or tCD19 (tCD19 control), or tCD19 and FLT3-specific CAR encoded by the nucleic acid molecule without optimization (Before Opt. FLT3/CD19), or tCD19 and FLT3-specific CAR encoded by the optimized nucleic acid molecule (After Opt. FLT3/CD19). And then each group of the engineered T cells was incubated with two cancer cell lines separately: MOLM13 cells are FLT3+ tumor target while U937 cells do not express FLT3 and served as a negative control. Accordingly, two bars are plotted in FIGS. 7A-7C for each Donor and each T cell group: the left one indicates IFNγ concentration in the co-culture with MOLM13, while the right one indicates IFNγ concentration in the co-culture with U937.

FIG. 8 shows that FLT3-specific CAR cells slow/halt AML progression in dose-dependent manner. FLT3-specific CAR expressing NK cells were compared with FLT3-specific CAR expressing T cells in a MOLM-13 model of FLT3(+) AML. All work was performed with viably frozen primary human CAR NK cells derived from cord blood.

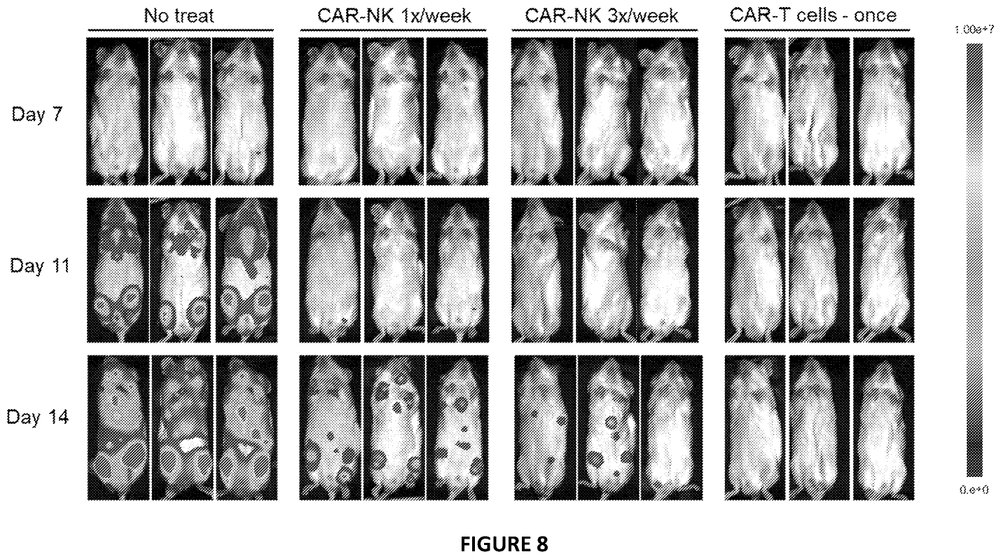

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“of”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

In some embodiments, the terms “first” “second” “third” “fourth” or similar in a component name are used to distinguish and identify more than one components sharing certain identity in their names. For example, “first administration” and “second administration” are used across the specification to distinguishing two administrations.

The terms “polynucleotide” “nucleic acid” and “nucleic acid molecule” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, and/or a reverse-complement (i.e., complementary) reference, optionally under conditions of high stringency. Additionally or alternatively, an equivalent nucleic acid, polynucleotide or oligonucleotide is one having at least 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement. In another aspect, an equivalent has at least the 70%, or at least 75%, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, or oligonucleotide, or alternatively an equivalent nucleic acid hybridizes under conditions of high stringency to a reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

In some embodiments, a coding nucleic acid molecule can be optimized, for example, to have a certain GC content. GC-content (or guanine-cytosine content) is the percentage of nitrogenous bases in a nucleic acid molecule that are either guanine (G) or cytosine (C). This measure indicates the proportion of G and C bases (i.e., G and C residues) out of an implied four total bases, also including adenine and thymine in DNA and adenine and uracil in RNA. Quantitatively, each GC base pair is held together by three hydrogen bonds, while AT and AU base pairs are held together by two hydrogen bonds. Accordingly, a nucleic acid molecule with low GC-content has a lower thermos-stability compared to that with high GC-content. It has also been demonstrated that the most important factor contributing to the thermal stability of double-stranded nucleic acids is actually due to the base stackings of adjacent bases. There is more favorable stacking energy for GC pairs than for AT or AU pairs because of the relative positions of exocyclic groups.

The term “codon” as used herein refer to a sequence of three consecutive nucleotides that corresponds with a specific amino acid residue or a stop signal during protein synthesis. In some embodiments, the codon is a standard genetic code as provided in the table below.

| 1st | 2nd base | | | | | | | | 3rd |
|---|---|---|---|---|---|---|---|---|---|
| base | T | | C | | A | | G | | base |
| T | TTT | Phe/ | TCT | Ser/S | TAT | Tyr/Y | TGT | Cys/C | T |
| | TTC | F | TCC | | TAC | | TGC | | C |
| | TTA | Leu/ | TCA | | TAA | Stop | TGA | Stop | A |
| | TTG | L | TCG | | TAG | Stop | TGG | Trp/W | G |
| C | CTT | | CCT | Pro/P | CAT | His/H | CGT | Arg/R | T |
| | CTC | | CCC | | CAC | | CGC | | C |
| | CTA | | CCA | | CAA | Gln/Q | CGA | | A |
| | CTG | | CCG | | CAG | | CGG | | G |
| A | ATT | Ile/I | ACT | Thr/T | AAT | Asn/N | AGT | Ser/S | T |
| | ATC | | ACC | | AAC | | AGC | | C |
| | ATA | | ACA | | AAA | Lys/K | AGA | Arg/R | A |
| | ATG | Met/ | ACG | | AAG | | AGG | | G |
| | | M | | | | | | | |
| G | GTT | Val/ | GCT | Ala/ | GAT | Asp/D | GGT | Gly/G | T |
| | GTC | V | GCC | A | GAC | | GGC | | C |
| | GTA | | GCA | | GAA | Glu/E | GGA | | A |
| | GTG | | GCG | | GAG | | GGG | | G |

The term codon frequency of a nucleic acid molecule refers to the rate of recurrence of a codon used by the nucleic acid molecule upon expressing a polypeptide. In some embodiments, a codon frequency as used herein is presented as the percentage of the number of the codon over the total codon number of the full nucleic acid molecule. In other embodiments, a codon frequency as used herein is presented as the average number of the codon per the 1000 codons. For example, a nucleic acid molecular having 300 nucleotide residues uses 100 codons, 10 of which are a codon consisting of GCC and encoding an alanine (Ala) amino acid residue. Thus, the GCC codon frequency may be presented as 10%, or 100 per 1000 codons. Several online tools are available for calculating codon frequency of a nucleic acid, such as www.bioinformatics.org/sms2/codon_usage.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

A “gene” refers to a polynucleotide containing at least one open reading frame (ORF) that is capable of encoding a particular polypeptide or protein after being transcribed and translated. The term “express” refers to the production of a gene product.

As used herein, the term “expression” refers to the process by which polynucleotides are transcribed into mRNA or the process by which the transcribed mRNA is subsequently being translated into peptides, polypeptides, or proteins, or both processes. If the polynucleotide is derived from genomic DNA, expression may include splicing of the mRNA in a eukaryotic cell. The expression level of a gene may be determined by measuring the amount of mRNA or protein in a cell or tissue sample.

The term “transduce” or “transduction” as it is applied to the production of CAR expressing cells refers to the process whereby a foreign nucleotide sequence is introduced into a cell. In some embodiments, this transduction is done via a vector.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. In one embodiment, the viral vector is a retroviral vector.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. In some embodiments, one or more plasmids are used in producing a viral vector or a viral genome. In some embodiments, a plasmid is used for replicating or amplifying a polynucleotide. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In embodiments where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing non-dividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” or “derived from” a particular virus means that the vector is derived from that particular virus. The genome of the vector particle comprises components from that virus as a backbone. In some embodiments, the virus is a retrovirus. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

The term “a regulatory sequence” “a regulatory element: “an expression control element” or “promoter” as used herein, intends a polynucleotide that is operatively linked to a target polynucleotide to be transcribed and/or replicated, and facilitates the expression and/or replication of the target polynucleotide.

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

A promoter is an example of an expression control element or a regulatory sequence. The term “promoter” as used herein refers to any sequence that regulates the expression of a coding sequence. Promoters can be located 5′ or upstream of a gene or other polynucleotide, that provides a control point for regulated gene transcription. Polymerase II and III are examples of promoters. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include an EF1alpha promoter, a Cytomegalovirus (CMV) promoter, and an MMLV promoter.

An EF1alpha (also referred to herein as EF-1alpha) promoter sequence is known in the art (see, e.g., addgene.org/11154/sequences/; ncbi.nlm.nih.gov/nuccore/J04617, each last accessed on Mar. 13, 2019, and Zheng and Baum (2014) Int'l. J. Med. Sci. 11(5):404-408). In some embodiments, an EF1alpha promoter comprises, or consists essentially of, or yet further consists of AAGGATCTGCGATCGCTCCGGTGCCCGTCAGTGGGCAGAGCGCACATCGCCCAC AGTCCCCGAGAAGTTGGGGGGAGGGGTCGGCAATTGAACGGGTGCCTAGAGAA GGTGGCGCGGGGTAAACTGGGAAAGTGATGTCGTGTACTGGCTCCGCCTTTTTCC CGAGGGTGGGGGAGAACCGTATATAAGTGCAGTAGTCGCCGTGAACGTTCTTTTT CGCAACGGGTTTGCCGCCAGAACACAGCTGAAGCTTCGAGGGGCTCGCATCTCT CCTTCACGCGCCCGCCGCCCTACCTGAGGCCGCCATCCACGCCGGTTGAGTCGCG TTCTGCCGCCTCCCGCCTGTGGTGCCTCCTGAACTGCGTCCGCCGTCTAGGTAAG TTTAAAGCTCAGGTCGAGACCGGGCCTTTGTCCGGCGCTCCCTTGGAGCCTACCT AGACTCAGCCGGCTCTCCACGCTTTGCCTGACCCTGCTTGCTCAACTCTACGTCTT TGTTTCGTTTTCTGTTCTGCGCCGTTACAGATCCAAGCTGTGACCGGCGCCTAC (SEQ ID NO: 23), and optionally, an equivalent thereof.

A cytomegalovirus (CMV) promoter sequence is also known in the art (see, e.g., snapgene.com/resources/plasmid-files/?set=basic_cloning_vectors&plasmid=CMV_promoter, last accessed on Mar. 13, 2019 and Zheng and Baum (2014), supra.). In some embodiments, an CMV promoter comprises, or consists essentially of, or yet further consists of

(SEQ ID NO: 30)
atcgattggctcatgtccaacattaccgccatgtt
gacattgattattgactagttattaatagtaatca
attacggggtcattagttcatagcccatatatgga
gttccgcgttacataacttacggtaaatggcccgc
ctggctgaccgcccaacgacccccgcccattgacg
tcaataatgacgtatgttcccatagtaacgccaat
agggactttccattgacgtcaatgggtggagtatt
tacggtaaactgcccacttggcagtacatcaagtg
tatcatatgccaagtacgccccctattgacgtcaa
tgacggtaaatggcccgcctggcattatgcccagt
acatgaccttatgggactttcctacttggcagtac
atctacgtattagtcatcgctattaccatggtgat
gcggttttggcagtacatcaatgggcgtggatagc
ggtttgactcacggggatttccaagtctccacccc
attgacgtcaatgggagtttgttttggcaccaaaa
tcaacgggactttccaaaatgtcgtaacaactccg
ccccattgacgcaaatgggcggtaggcgtgtacgg
aatt.

A moloney murine leukemia virus (MMLV) promoter sequence is known in the art (see, e.g., Lorens et al. Virology. 2000 Jun. 20; 272(1):7-15). In some embodiments, an EF1alpha promoter comprises, or consists essentially of, or yet further consists of SEQ ID NO: 24.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acid residues and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term equivalent and biological equivalent are used interchangeably, for example when referring to a protein or polypeptide as a reference. In some embodiments, an equivalent protein or polypeptide is one having at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to a polypeptide or protein as disclosed herein. In some embodiments, an equivalent protein or polypeptide has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to polypeptide or protein encoded by an equivalent polynucleotide as noted herein. In addition or alternatively, the equivalent of a polynucleotide would encode a protein or polypeptide of the same or similar function as the reference or parent polynucleotide. In some embodiments, the equivalent is a functional protein that optionally can be identified through one or more assays described herein or otherwise available to one of skill in the art. In another aspect, an equivalent has at least about 60%, at least about 70%, at least about 80%, at least about 85%, at least about 90%, at least about 91%, at least about 92%, at least about 93%, at least about 94%, or at least about 95%, at least about 96%, at least about 97%, at least about 98%, or at least about 99% sequence identity to the reference protein or polypeptide.

As used herein, an amino acid (aa) or nucleotide (nt) residue position in a sequence of interest “corresponding to” or “aligned to” an identified position in a reference sequence refers to that the residue position is aligned to the identified position in a sequence alignment between the sequence of interest and the reference sequence. Various programs are available for performing such sequence alignments, such as Clustal Omega and BLAST.

As used herein, the term “specifically recognizing and binding” means the contact between an antibody (or an antigen binding fragment thereof, or a biological agent comprising such antibody or antigen binding fragment, such as a CAR or a CAR expressing cell) and its target antigen with a binding affinity substantially higher than the binding affinity with another molecule. In some embodiments, the binding affinity between the antibody (or an antigen binding fragment thereof, or a biological agent comprising such antibody or antigen binding fragment, such as a CAR or a CAR expressing cell) and its target antigen is at least 10⁻³M, including any number or any range therein, such as at least about 10⁻⁶ M, or at least about 10⁻⁷ M, and preferably 10⁻⁸ M, 10⁻⁹ M, 10⁻¹⁰ M, 10⁻¹¹ M, or 10⁻¹² M.

As used herein, the terms “FLT3” and “FMS-like tyrosine kinase 3” are used interchangeably, referring to a receptor-type tyrosine-protein kinase FLT3 associated with this name, any of its alternate names (Fms-Related Tyrosine Kinase, Stem Cell Tyrosine Kinase, Fms-Like Tyrosine Kinase, FL Cytokine Receptor, CD135 Antigen, EC 2.7.10.1, CD135, FLK-2, STK1, FLK2, Growth Factor Receptor Tyrosine Kinase Type III, Receptor-Type Tyrosine-Protein Kinase FLT3, Fetal Liver Kinase 2, Fetal Liver Kinase-2, EC 2.7.10, FLT-3, or STK-1) or UniProt Accession No. P36888 and any other molecules that have analogous biological function that share at least 80% amino acid sequence identity, preferably 90% sequence identity, or alternatively at least 95% sequence identity with FLT3 and any variant or isoform thereof. Monoclonal antibodies that specifically bind FLT3 are commercially available from, for example, Becton Dickinson Biosciences and other commercial sources, e.g., those listed at the web address: biocompare.com/Search-Antibodies/?search=FLT3&said=0. Methods to prepare antigen binding fragments are known in the art. The antigen binding domains may be from any appropriate species, e.g., sheep or human.

Non-limiting examples of FLT3 include: Human FLT3 Isoform 1 consisting of MPALARDGGQLPLLVVFSAMIFGTITNQDLPVIKCVLINHKNNDSSVGKSSSYPMVSE SPEDLGCALRPQSSGTVYEAAAVEVDVSASITLQVLVDAPGNISCLWVFKHSSLNCQ PHFDLQNRGVVSMVILKMTETQAGEYLLFIQSEATNYTILF TVSIRNTLLYTLRRPYF RKMENQDALVCISESVPEPIVEWVLCDSQGESCKEESPAVVKKEEKVLHELFGTDIR CCARNELGRECTRLF TIDLNQTPQTTLPQLFLKVGEPLWIRCKAVHVNHGFGLTWEL ENKALEEGNYFEMSTYSTNRTMIRILFAFVSSVARNDTGYYTCSSSKHPSQSALVTIV EKGFINATNSSEDYEIDQYEEFCFSVRFKAYPQIRCTWTFSRKSFPCEQKGLDNGYSIS KFCNHKHQPGEYIFHAENDDAQFTKMFTLNIRRKPQVLAEASASQASCFSDGYPLPS WTWKKCSDKSPNCTEEITEGVWNRKANRKVFGQWVSSSTLNMSEAIKGFLVKCCA YNSLGTSCETILLNSPGPFPFIQDNISFYATIGVCLLFIVVLTLLICHKYKKQFRYESQL QMVQVTGSSDNEYFYVDFREYEYDLKWEFPRENLEFGKVLGSGAFGKVMNATAYG ISKTGVSIQVAVKMLKEKADSSEREALMSELKMMTQLGSHENIVNLLGACTLSGPIY LIFEYCCYGDLLNYLRSKREKFHRTWTEIFKEHNFSFYPTFQSSHPNSSMPGSREVQIHP DSDQISGLHGNSFHSEDEIEYENQKRLEEEEDLNVLTFEDLLCFAYQVAKGMEFLEFK SCVHRDLAARNVLVTHGKVVKICDFGLARDIMSDSNYVVRGNARLPVKWMAPESL FEGIYTIKSDVWSYGILLWEIFSLGVNPYPGIPVDANFYKLIQNGFKMDQPFYATEEIY IIMQSCWAFDSRKRPSFPNLTSFLGCQLADAEEAMYQNVDGRVSECPHTYQNRRPFS REMDLGLLSPQAQVEDS (SEQ ID NO: 25), and optionally an equivalent thereof, and Human FLT3 Isoform 2 consisting of MPALARDGGQLPLLVVFSAMIFGTITNQDLPVIKCVLINHKNNDSSVGKSSSYPMVSE SPEDLGCALRPQSSGTVYEAAAVEVDVSASITLQVLVDAPGNISCLWVFKHSSLNCQ PHFDLQNRGVVSMVILKMTETQAGEYLLFIQSEATNYTILF TVSIRNTLLYTLRRPYF RKMENQDALVCISESVPEPIVEWVLCDSQGESCKEESPAVVKKEEKVLHELFGTDIR CCARNELGRECTRLF TIDLNQTPQTTLPQLFLKVGEPLWIRCKAVHVNHGFGLTWEL ENKALEEGNYFEMSTYSTNRTMIRILFAFVSSVARNDTGYYTCSSSKHPSQSALVTIV EKGFINATNSSEDYEIDQYEEFCFSVRFKAYPQIRCTWTFSRKSFPCEQKGLDNGYSIS KFCNHKHQPGEYIFHAENDDAQFTKMFTLNIRRKPQVLAEASASQASCFSDGYPLPS WTWKKCSDKSPNCTEEITEGVWNRKANRKVFGQWVSSSTLNMSEAIKGFLVKCCA YNSLGTSCETILLNSPGPFPFIQDNISFYATIGVCLLFIVVLTLLICHKYKKQFRYESQL QMVQVTGSSDNEYFYVDFREYEYDLKWEFPRENLEFGKVLGSGAFGKVMNATAYG ISKTGVSIQVAVKMLKEKADSSEREALMSELKMMTQLGSHENIVNLLGACTLSGPIY LIFEYCCYGDLLNYLRSKREKFHRTWTEIFKEHNFSFYPTFQSSHPNSSMPGSREVQIHP DSDQISGLHGNSFHSEDEIEYENQKRLEEEEDLNVLTFEDLLCFAYQVAKGMEFLEFK SARLPVKWMAPESLFEGIYTIKSDVWSYGILLWEIFSLGVNPYPGIPVDANFYKLIQN GFKMDQPFYATEEIYIIMQSCWAFDSRKRPSFPNLTSFLGCQLADAEEAMYQNVDGR VSECPHTYQNRRPFSREMDLGLLSPQAQVEDS (SEQ ID NO: 26), and optionally an equivalent thereof.

As used herein, an activating mutation in Fms-like tyrosine kinase 3 (FLT3) refers to a mutant FLT3 nucleotide sequence or amino acid sequence leading to an activation of the FLT3 kinase, such as a FLT3 internal tandem duplication (ITD) mutation. FLT3 is mutated in about one third of acute myeloid leukemia cases. The most frequent FLT3 mutations in acute myeloid leukemia are internal tandem duplication (ITD) mutations in the juxtamembrane domain (23%) and point mutations in the tyrosine kinase domain (10%). The most frequent kinase domain mutation is the substitution of aspartic acid at position 838 (equivalent to the human aspartic acid residue at position 835) with a tyrosine (FLT3/D835Y), converting aspartic acid to tyrosine. Even though both of these mutations constitutively activate FLT3, patients with an ITD mutation have a much poorer prognosis. Also, see more details at U.S. Pat. No. 6,846,630, which is incorporated herein by reference in its entirety.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).”

The “extracellular domain capable of binding to an antigen” means any oligopeptide or polypeptide that can bind to a certain antigen, such as an antibody or an antigen binding fragment thereof.

As used herein, the term “antibody” (also referred to herein as “immunoglobulin”) collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ or greater, at least 10⁴ M⁻¹ or greater or at least 10⁵ M⁻¹ or greater than a binding constant for other molecules in a biological sample). The term “antibody” also includes genetically engineered forms such as chimeric antibodies (for example, murine or humanized non-primate antibodies), or heteroconjugate antibodies (such as, bispecific antibodies), or both. See also, Pierce Catalog and Handbook, 1994-1995 (Pierce Chemical Co., Rockford, Ill.); Owen et al., Kuby Immunology, 7^{th} Ed., W.H. Freeman & Co., 2013; Murphy, Janeway's Immunobiology, 8^{th} Ed., Garland Science, 2014; Male et al., Immunology (Roitt), 8^{th} Ed., Saunders, 2012; Parham, The Immune System, 4^{th} Ed., Garland Science, 2014.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDRH and light chain regions labeled CDRL). Thus, a CDRH3 is the CDR3 from the variable domain of the heavy chain of the antibody in which it is found, whereas a CDRL1 is the CDR1 from the variable domain of the light chain of the antibody in which it is found. For example, an FLT3 antibody will have a specific VH region and the VL region sequence unique to the FLT3 relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

In some embodiments, antigen of a binding moiety, such as an antibody, an antigen binding fragment thereof, or a CAR, may be provided herein in a format of “antigen” followed by the binding moiety (such as an FLT3 CAR or an FLT3-specific CAR), or having “anti-” before the antigen and the binding moiety after the antigen (such as an anti-FLT3 antibody), or the binding moiety followed by “to” or “directed to” and then the antigen (such as an antibody to FLT3).

As used herein, the term “antigen binding domain” refers to any protein or polypeptide domain that can specifically bind to an antigen target, such as FLT3. In some embodiments, the antigen binding fragment may be selected from the group consisting of Fab, F(ab′)₂, Fab′, scFv, or Fv.

A single chain Fv fragment (scFv) comprises, or consists essentially of, or yet further consists of a heavy chain variable region and a light chain variable region connected with a linker peptide (typically around 5 to 25 amino acids in length). In the scFv, the variable regions of the heavy chain and the light chain may be derived from the same antibody or different antibodies. In some embodiments, an FLT3 scFv is disclosed herein as SEQ ID NO: 4. Additional suitable FLT3 scFv can be found, for example, in U.S. Pat. No. 10,961,312 and WO2020/010284, each of which is incorporated herein by reference in its entirety.

In some embodiments, the terms “linker sequence” “linker peptide” and “linker polypeptide” are used interchangeably, relating to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In further embodiments, the linker sequence is a (Glycine₄Serine)₃ (aa 119 to aa 133 of SEQ ID NO: 4) flexible polypeptide linker comprising three copies of Gly-Gly-Gly-Gly-Ser (SEQ ID NO: 27). In other embodiments, the terms “linker sequence” “linker peptide” and “linker polypeptide” refer to a peptide consists of some (for example, about 1 to about 50) random amino acid residues.

In some embodiments, the term “equivalent” or “biological equivalent” of an antibody means the ability of the antibody to selectively bind its epitope/antigen protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

The “transmembrane domain” means any oligopeptide or polypeptide known to span the cell membrane and that can function to link the extracellular and signaling domains. The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, and TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker. In some embodiments, the transmembrane domain comprises, or alternatively consists essentially of, or yet consists of a CD8α transmembrane domain or a CD28 transmembrane domain.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are incorporated herein by reference in its entirety. In some embodiments, the CD28 transmembrane domain comprises, or alternatively consists essentially of, or yet consists of SEQ ID NO: 6 or an equivalent thereof. In further embodiments, the equivalent of SEQ ID NO: 6 may comprises 1, or 2, or 3, or 4, or 5, or 6, or 7, or 8, or 9, or 10, or 11, or 12, or 13, or 14, or 15, or more mutations compared to SEQ ID NO: 6 but is still capable of spanning the cell membrane and functioning to link the extracellular and signaling domains as SEQ ID NO: 6.

The “intracellular domain” “intracellular signaling domain” or “cytoplasmic domain” means any oligopeptide or polypeptide known to function as a domain that transmits a signal to cause activation or inhibition of a biological process in a cell. The intracellular signaling domain of the CAR is responsible for activation of at least one of the traditional effector functions of an immune cell in which a CAR has been placed. In some embodiments, the intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, and CD66d. In some embodiments, the intracellular signaling domain of the CAR can comprise, or alternatively consist essentially of, or yet consist of a CD3 (i.e., CD3zeta) signaling domain.

As used herein, the term “CD3 zeta signaling domain” or “CD3 zeta intracellular signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. In some embodiments, the CD3 zeta signaling domain comprises, or alternatively consists essentially of, or yet consists of RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR (SEQ ID NO: 8) or an equivalent thereof. In further embodiments, the equivalent of SEQ ID NO: 8 may comprises 1, or 2, or 3, or 4, or 5, or 6, or 7, or 8, or 9, or 10, or 11, or 12, or 13, or 14, or 15, or more mutations compared to SEQ ID NO: 8 but is still capable of transducing the effector function signal and directing the immune cell to perform its specific function as SEQ ID NO: 8. Exemplified methods assessing such transduction can be found, for example, in Bridgeman J S, et al. Clin Exp Immunol. 2014 February; 175(2):258-67.

Since signals generated through the TCR are alone insufficient for full activation of a T cell, a secondary or co-stimulatory signal may also be required. Thus, the intracellular region of a co-stimulatory signaling molecule (also referred to herein as a costimulatory domain or a costimulatory signaling domain or a costimulatory signaling region), including but not limited the intracellular domains of the proteins CD27, CD28, 4-IBB (CD 137), OX40, CD30, CD40, PD-1, ICOS (CD278), lymphocyte function-associated antigen-1 (LFA-1), CD2, CD7, LIGHT, NKG2C, NKG2D, DAP10, DAP12, 2B3, 4B2, B7-H3, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, ICAM-1, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, or a ligand that specifically binds with CD83, may also be included in the cytoplasmic domain of the CAR and referred to herein as a costimulatory signaling domain. In certain embodiments, the intracellular domain may comprise, alternatively consist essentially of, or yet further comprise one or more costimulatory signaling domains in addition to the primary signaling domain. For instance, a CAR may comprise one, two, or more costimulatory domains, in addition to a signaling domain (e.g., a CD3 (signaling domain). In some embodiments, the intracellular domain further comprises one or more or two or more costimulatory regions selected from a CD28 costimulatory signaling region, a 4-1BB costimulatory signaling region, an ICOS costimulatory signaling region, or an OX40 costimulatory region.

In some embodiments, the cell activation moiety (e.g., the cytoplasmic region) of the chimeric antigen receptor is a T cell or an NK cell signaling domain comprising, or alternatively consisting essentially of, or yet further consisting of, one or more proteins or fragments thereof selected from the group consisting of CD8 protein, CD28 protein, 4-1BB protein, OX40, CD30, CD40, PD-1, ICOS, LFA-1, CD2, CD7, CD27, LIGHT, NKG2C, B7-H3, and CD3-zeta protein.

In some embodiments, the cell activation moiety (e.g., the cytoplasmic region) of the chimeric antigen receptor is a T cell or an NK cell signaling domain comprising, or alternatively consisting essentially of, or yet further consisting of, one or more proteins or fragments thereof selected from the group consisting of CD8 protein, CD28 protein, 4-1BB protein, and CD3zeta protein.

As used herein, the term “CD28 costimulatory signaling region” or a “CD28 costimulatory domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). In some embodiments, the CD28 costimulatory domain comprises, or alternatively consists essentially of, or yet consists of SEQ ID NO: 7 or an equivalent thereof. In further embodiments, the equivalent of SEQ ID NO: 7 may comprises 1, or 2, or 3, or 4, or 5, or 6, or 7, or 8, or 9, or 10, or 11, or 12, or 13, or 14, or 15, or more mutations compared to SEQ ID NO: 7 but is still capable of stimulating the immune cell to perform its specific function as SEQ ID NO: 7.

A signal peptide (sometimes referred to as signal sequence, targeting signal, localization signal, localization sequence, transit peptide, leader sequence or leader peptide) is a short peptide (usually 16-30 amino acids long) present at the N-terminus of the majority of newly synthesized proteins that are destined toward the secretory pathway. These synthesized proteins are the directed to reside either inside certain organelles (the endoplasmic reticulum, Golgi or endosomes), secreted from the cell, or inserted into most cellular membranes. In some embodiments, a signal peptide as disclosed herein direct the protein to be expressed on the cell membrane.

A chimeric antigen receptor may optionally comprise a “hinge domain” which serves as a linker between the extracellular and transmembrane domains.

A spacer domain (also referred to herein as a spacer or a hinge or a hinge domain) is an extracellular structural region of the CAR that separates the binding units from the transmembrane domain. These spacers generally supply stability for efficient CAR expression and activity. Such a domain may comprise, or consist essentially of, or yet further consist of, for example, a portion of a human Fc domain, a CH3 domain, or the hinge region of any immunoglobulin, such as IgA, IgD, IgE, IgG, or IgM, or variants thereof. For example, some embodiments may comprise an IgG4 hinge with or without a S228P, L235E, and/or N297Q mutation (according to Kabat numbering). Additional spacers include, but are not limited to, CD4, CD8, and CD28 hinge regions. The hinge domain may be derived either from a natural or from a synthetic source. In some embodiments, the hinge domain is derived from a cluster of differentiation protein, such as CD8, CD28, CD3, CD45, CD4, CD5, CDS, CD9, CD 16, CD22, CD33, CD37, CD64, CD80, CD86, CD 134, CD137, CD 154. In one embodiments, the hinge domain is a CD8α hinge domain. In some embodiments, the hinge domain is derived from an immunoglobulin, such as IgA, IgD, IgE, IgG, or IgM. In one embodiment, the hinge domain is an IgG1 hinge domain. In a further embodiment, the IgG1 hinge domain comprises, or alternatively consists essentially of, or yet consists of LEPKSCDKTHTCPPCPDPKGT (SEQ ID NO: 5) or an equivalent thereof. In some embodiments, an equivalent of SEQ ID NO: 5 comprises 1, or 2, or 3, or 4, or 5, or 6, or 7, or 8, or 9, or 10, or 11, or 12, or 13, or 14, or 15, or more mutations compared to SEQ ID NO: 5 but is still capable of substantially maintaining the stability for efficient CAR expression and activity as SEQ ID NO: 5.

A protein expressed on cell surface may be used as a marker or to provide a suicide switch of a CAR expressing cell as disclosed herein. A portion of or the whole cytoplasmic region of such protein is usually truncated so that the native function of the protein is reduced or even abolished. Thus, such a protein is referred to herein as a truncated protein marker. In some embodiments, when used as a suicide switch of the CAR expressing cell, the truncated protein marker does not express or is expressed at a substantially lower level on a normal cell or a normal cell adjacent to the CAR expressing cell in the subject. Accordingly, upon removal of the CAR expressing cell (for example, by administering a neutralizing antibody specially recognizing and binding the truncated protein marker, or by administering a toxin conjugated to a moiety directing the toxin to the truncated protein marker,), a normal cell of the subject would not be jeopardized.

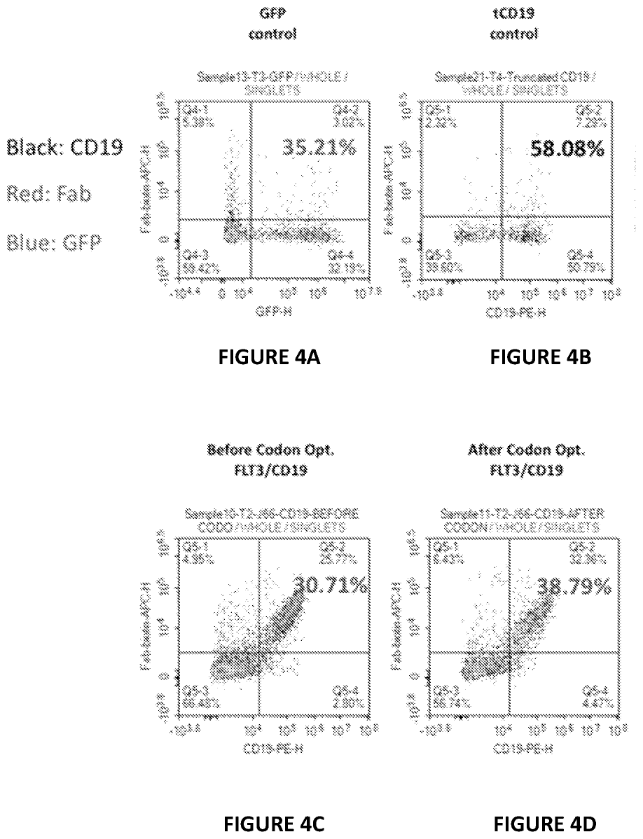

In some embodiments, the disclosure herein shows that certain truncated protein markers (for example, a truncated CD19, or a truncated EGFR) are expressed at a higher rate compared to other markers (such as a green fluorescent protein (GFP)). See, FIG. 4B versus. FIG. 4A. In further embodiments, Thus, those truncated protein markers are particular suitable for using in a CAR expressing cell as disclosed herein.

One of such suitable truncated protein markers is a truncated CD19. As used herein the terms “CD19,” and “B-lymphocyte antigen CD19” are used interchangeably to refer to a protein known to be a transmembrane protein that in humans is encoded by the gene CD19. In humans, CD19 is expressed in all B lineage cells, except for plasma cells, and in follicular dendritic cells. Non-limiting exemplary sequences of this protein or the underlying gene may be found under Gene Cards ID: GC16PO28943, HGNC: 1633, Entrez Gene: 930, Ensembl: ENSG00000177455, OMIM: 107265, and UniProtKB: P15391, which are incorporated by reference herein. An exemplified truncated CD19 is provided herein and comprising, or consisting essentially of, or yet further consisting of

(SEQ ID NO: 13)
MPPPRLLFFLLFLTPMEVRPEEPLVVKVEEGDNAV
LQCLKGTSDGPTQQLTWSRESPLKPFLKLSLGLPG
LGIHMRPLAIWLFIFNVSQQMGGFYLCQPGPPSEK
AWQPGWTVNVEGSGELFRWNVSDLGGLGCGLKNRS
SEGPSSPSGKLMSPKLYVWAKDRPEIWEGEPPCVP
PRDSLNQSLSQDLTMAPGSTLWLSCGVPPDSVSRG
PLSWTHVHPKGPKSLLSLELKDDRPARDMWVMETG
LLLPRATAQDAGKYYCHRGNLTMSFHLEITARPVL
WHWLLRTGGWKVSAVTLAYLIFCLCSLVGILHLQR
ALVLRRKR.

Another exemplified truncated CD19 is provided herein and comprising, or consisting essentially of, or yet further consisting of MPPPRLLFFLLFLTPMEVRPEEPLVVKVEEGDNAVLQCLKGTSDGPTQQLTWSRESP LKPFLKLSLGLPGLGIHMRPLAIWLFIFNVSQQMGGFYLCQPGPPSEKAWQPGWTVN VEGSGELFRWNVSDLGGLGCGLKNRSSEGPSSPSGKLMSPKLYVWAKDRPEIWEGE PPCLPPRDSLNQSLSQDLTMAPGSTLWLSCGVPPDSVSRGPLSWTH (SEQ ID NO: 11). Alternatively, an equivalent of SEQ ID NO: 13 or 11, for example those comprising 1, or 2, or 3, or 4, or 5, or 6, or 7, or 8, or 9, or 10, or 11, or 12, or 13, or 14, or 15, or more mutations compared to SEQ ID NO: 13 or 11 or those having at least 90%, at least 95%, at least 98% identical to SEQ ID NO: 13 or 11, may be used. In further embodiments, an equivalent of SEQ ID NO: 13 or 11 is still capable of being recognized and bound to by a moiety, such as an antibody or an antigen binding fragment thereof, specifically recognizing and binding CD19. In yet further embodiments, the equivalent of SEQ ID NO: 13 or 11 does not direct a cell expressing the equivalent to perform a function as a wild type CD19 does. In some embodiments, the polynucleotide encoding SEQ ID NO: 11 comprises or consists essentially of or et further consists of

(SEQ ID NO: 28)
ATGCCACCTCCTCGCCTCCTCTTCTTCCTCCTCTT
CCTCACCCCCATGGAAGTCAGGCCCGAGGAACCTC
TAGTGGTGAAGGTGGAAGAGGGAGATAACGCTGTG
CTGCAGTGCCTCAAGGGGACCTCAGATGGCCCCAC
TCAGCAGCTGACCTGGTCTCGGGAGTCCCCGCTTA
AACCCTTCTTAAAACTCAGCCTGGGGCTGCCAGGC
CTGGGAATCCACATGAGGCCCCTGGCCATCTGGCT
TTTCATCTTCAACGTCTCTCAACAGATGGGGGGCT
TCTACCTGTGCCAGCCGGGGCCCCCCTCTGAGAAG
GCCTGGCAGCCTGGCTGGACAGTCAATGTGGAGGG
CAGCGGGGAGCTGTTCCGGTGGAATGTTTCGGACC
TAGGTGGCCTGGGCTGTGGCCTGAAGAACAGGTCC
TCAGAGGGCCCCAGCTCCCCTTCCGGGAAGCTCAT
GAGCCCCAAGCTGTATGTGTGGGCCAAAGACCGCC
CTGAGATCTGGGAGGGAGAGCCTCCGTGTcTCCCA
CCGAGGGACAGCCTGAACCAGAGCCTCAGCCAGGA
CCTCACCATGGCCCCTGGCTCCACACTCTGGCTGT
CCTGTGGGGTACCCCCTGACTCTGTGTCCAGGGGC
CCCCTCTCCTGGACCCAT.

Another suitable truncated protein marker is a truncated EGFR. As used herein the terms “EGFR” and “Epidermal Growth Factor Receptor” are used interchangeably to refer to a protein known to be a transmembrane protein that in humans is encoded by the gene EGFR. Non-limiting exemplary sequences of this protein or the underlying gene may be found under Gene Cards ID: GC07P055019, HGNC: 3236, NCBI Entrez Gene: 1956, Ensembl: ENSG00000146648, OMIM®: 131550, UniProtKB/Swiss-Prot: P00533, each of which is incorporated by reference herein by its entirety. An exemplified truncated EGFR is provided herein and comprising, or consisting essentially of, or yet further consisting of LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 10). Alternatively, an equivalent of SEQ ID NO: 10, for example those comprising 1, or 2, or 3, or 4, or 5, or 6, or 7, or 8, or 9, or 10, or 11, or 12, or 13, or 14, or 15, or more mutations compared to SEQ ID NO: 10 or those having at least 90%, at least 95%, at least 98% identical to SEQ ID NO: 10, may be used. In further embodiments, an equivalent of SEQ ID NO: 10 is still capable of being recognized and bound to by a moiety, such as an antibody or an antigen binding fragment thereof, specifically recognizing and binding EGFR. In yet further embodiments, the equivalent of SEQ ID NO: 10 does not direct a cell expressing the equivalent to perform a function as a wild type EGFR does.

As used herein, a ribosomal skip sequence which is also referred to as a cleavable peptide, or a cleavable linker, means a peptide that can be cleaved, for example, by an enzyme or a peptide that can induce ribosomal skipping during translation of a protein in a cell, or both. One translated polypeptide comprising such cleavable peptide can produce two final products, therefore, allowing expressing more than one polypeptides from one open reading frame. Accordingly, a ribosomal skip sequence can be used to express a CAR and a truncated protein marker on a cell as disclosed herein. Alternatively, other methods can be used to achieve such co-expression, for example, by a nucleic acid molecule comprising a CAR coding sequence, a truncated protein marker coding sequence, an internal ribosome entry site (IRES) located therebetween.

One example of cleavable peptides is a self-cleaving peptide, such as a 2A self-cleaving peptide. 2A self-cleaving peptides, is a class of 18-22 aa-long peptides, which can induce the cleaving of the recombinant protein in a cell. In some embodiments, the 2A self-cleaving peptide is selected from P2A, T2A, E2A, F2A and BmCPV2A. See, for example, Wang Y, et al. 2A self-cleaving peptide-based multi-gene expression system in the silkworm Bombyx mori. Sci Rep. 2015; 5:16273. Published 2015 Nov. 5.

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P (SEQ ID NO: 29), wherein X refers to any amino acid generally thought to be self-cleaving.

The term “internal ribosome entry site” or “IRES” as used herein interchangeably refers to a polynucleotide that directly promotes ribosome binding and mRNA translation and thereby permits initiation of translation in cap-independent manner. In some embodiments, an IRES refers an RNA sequence on a messenger RNA (mRNA). Additionally or alternatively, an IRES also refers to a polynucleotide sequence (such as an RNA sequence, a DNA sequence or a hybrid thereof) complementary, or reverse, or both complementary and reverse to an IRES RNA sequence. Non-limiting examples of IRES can be found in Hellen CU and Sarnow P. Internal ribosome entry sites in eukaryotic mRNA molecules. Genes Dev. 2001 Jul. 1; 15(13):1593-612.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, CASCADE BLUE™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

The polypeptide or equivalents of each thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

Further embodiments of each CAR component as exemplified herein include other proteins that have analogous biological function that share at least 70%, or alternatively at least 80% amino acid sequence identity, preferably 90% sequence identity, more preferably at least 95% sequence identity with the exemplified component.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or residues) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Another preferred alignment program is Clustal Omega accessible at www.ebi.ac.uk/Tools/msa/clustalo/ or a Pairwise Sequence Alignment accessible at www.ebi.ac.uk/Tools/psa/, using default parameters.

As used herein, a cell may be a prokaryotic or a eukaryotic cell. In further embodiments, the cell is an immune cell.

“Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

“Immune cells” includes, e.g., white blood cells (leukocytes, such as granulocytes (neutrophils, eosinophils, and basophils), monocytes, and lymphocytes (T cells, B cells, natural killer (NK) cells and NKT cells)), lymphocytes (T cells, B cells, natural killer (NK) cells) and myeloid-derived cells (neutrophil, eosinophil, basophil, monocyte, macrophage, dendritic cells). In some embodiments, the immune cell is derived from hematopoietic stem cells (HSCs). In further embodiments, the HSCs are produced in the bone marrow. In yet further embodiments, the HSCs are isolated from cord blood. In other embodiments, the HSCs are isolated from peripheral blood. some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSc), hematopoietic stem cells (HSCs), or immortalized cells. In some embodiments, the HSC are derived from umbilical cord blood of a subject, peripheral blood of a subject, or bone marrow of a subject.

In some embodiments, immune cells refer to central memory T cells, NK cells, native memory T cells, pan T cells, or any combination thereof. In some embodiments, immune cells refer to peripheral blood mononuclear cells (PBMCs) substantially depleted for CD25+ cells and CD14+ cells.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™) TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (www.atcc.org/) and the German Collection of Microorganisms and Cell Cultures (www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (www.atcc.org/) and the German Collection of Microorganisms and Cell Cultures (www.dsmz.de/).

The term “stem cell” refers to a cell that is in an undifferentiated or partially differentiated state and has the capacity for self-renewal or to generate differentiated progeny or both. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural or neuronal stem cells (NSCs). In some embodiments, the stem or progenitor cells can be embryonic stem cells or an induced pluripotent stem cell (iPSC). In some embodiments, the stem or progenitor cells are hematopoietic stem cells (HSCs). As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, immune or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers.

A “precursor” or “precursor cell” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

As used herein, a “pluripotent cell” defines a less differentiated cell that can give rise to at least two distinct (genotypically or phenotypically or both) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, the term “pluripotent gene or marker” intends an expressed gene or protein that has been correlated with an immature or undifferentiated phenotype, e.g., Oct 3/4, Sox2, Nanog, c-Myc and LIN-28. Methods to identify such are known in the art and systems to identify such are commercially available from, for example, EMD Millipore (MILLIPLEX® Map Kit).

As used herein, hematopoietic stem cells (HSCs) are cells, such as stem cells, that give rise to all types of blood cells, including but not limited to white blood cells, red blood cells, and platelets. Hematopoietic stem cells can be found in the peripheral blood and the bone marrow. In some embodiments, an immune cell as disclosed herein is derived from an HSC.

The term “phenotype” refers to a description of an individual's trait or characteristic that is measurable and that is expressed only in a subset of individuals within a population. In some embodiments of the disclosure, an individual's phenotype includes the phenotype of a single cell, a substantially homogeneous population of cells, a population of differentiated cells, or a tissue comprised of a population of cells.

In some embodiments, a population of cells as described herein is substantially homogeneous. As used herein, “substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively more than 95%, of the cells are of the same or similar phenotype. Phenotype can be determined by a pre-selected cell surface marker or other marker.

The term “isolated” as used herein refers to molecules, biologicals, cellular materials, cells or biological samples being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

A population of cells intends a collection of more than one cell that is identical (clonal) or non-identical in phenotype or genotype or both. The population can be purified, highly purified, substantially homogenous or heterogeneous as described herein.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, the term “purified” does not require absolute purity; rather, it is intended as a relative term. Thus, for example, a purified nucleic acid, peptide, protein, biological complexes, cell or other active compound is one that is isolated in whole or in part from proteins or other contaminants. Generally, substantially purified peptides, proteins, biological complexes, cell or other active compounds for use within the disclosure comprise more than 80% of all macromolecular species present in a preparation prior to admixture or formulation of the peptide, protein, biological complex, cell or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

As used herein, the term “autologous,” in reference to cells refers to cells that are isolated and infused back into the same subject (recipient or host). “Allogeneic” refers to non-autologous cells.

A “composition” as used herein, refers to an active agent, such as a compound as disclosed herein and a carrier, inert or active. The carrier can be, without limitation, solid such as a bead or resin, or liquid, such as phosphate buffered saline.

A “pharmaceutical composition” is intended to include the combination of an active polypeptide, polynucleotide, antibody or a cell with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

Administration or treatment in “combination” refers to administering two agents such that their pharmacological effects are manifest at the same time. Combination does not require administration at the same time or substantially the same time, although combination can include such administrations. Such additional agents are described herein, e.g., cytoreductive therapy.

“Cytoreductive therapy,” as used herein, includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m² to not more than about 50 J/m², usually about 10 J/m².

As used herein, a combined therapy may be a drug increasing expression of FLT3 on a cancer cell. In some embodiments, the drug comprises an FLT3 inhibitor. As used herein, the term “FLT3 inhibitor” refers to a molecule that binds FLT3 and decreases its activity. Not to be bound by theory, it is believed that such FLT3 inhibitors can increase surface FLT3 expression on cells. Non-limiting examples of FLT3 inhibitors include gilteritinib (Astellas, CID 49803313), quizaritinib (Ambit Biosciences, CID 24889392), midostaurin (Novartis, CID 9829523), sorafenib (Bayer and Onxy Pharmaceuticals, CID 216239), sunitinib (Pfizer, CID 5329102), lestarutinib (Cephalon, CID 126565), FF-10101 (Fuijfilm), dovitinib (Novartis or Oncology Venture, CID 9886808), and equivalents thereof such as but not limited to salts and hydrates, for example, Gilteritinib Fumarate (CID 76970819), Quizartinib Dihydrochloride (CID 25184035), Midostaurin Hydrate (CID 71311854), Sorafenib tosylate (CID 406563), Sorafenib sulphate (CID 86672519), Sorafenib hydrobromide (CID 44599974), Sorafenib hydrochloride (CID 44599975), Sunitinib Malate (CID 6456015), Lestarutinib Hydrate (CID 45111934), Lestarutinib Methanolate (CID 131738508), Dovitinib Lactate (CID 44150621), Dovitinib Dilactic Acid (CID 66553150).

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “contacting” means direct or indirect binding or interaction between two or more molecules or other entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include intravenous, intracranial, oral administration, intraperitoneal, infusion, nasal administration, inhalation, injection, and topical application. In some embodiments, the administration is an intratumoral administration, or administration to a tumor microenvironment, or both. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

“Administration” can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. In some embodiments, 1×10⁴ to 1×10¹⁵ or ranges in between of cells as disclosed herein are administrated to a subject, such as 1×10⁷ to 1×10¹⁰. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

An agent of the present disclosure can be administered for therapy by any suitable route of administration. It will also be appreciated that the optimal route will vary with the condition and age of the recipient, and the disease being treated.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the term “disease” or “disorder” as used herein refers to a cancer or tumor (which are used interchangeably), a status of being diagnosed with such disease, a status of being suspect of having such disease, or a status of at high risk of having such disease. The disease can be a primary, recurrent, recalcitrant or metastatic cancer.

In some embodiments, the disease is a cancer. In further embodiments, the cancer is a leukemia or a lymphoma. As used herein a “leukemia” is a cancer of the blood or bone marrow characterized by an abnormal increase of immature white blood cells. In yet further embodiments, the disease is having a cancer cell expressing FLT3. In certain embodiments, the cancer is acute myeloid leukemia (AML) or acute lymphoblastic leukemia (ALL). The specific condition of acute myeloid leukemia (AML)—also referred to as acute myelogenous leukemia or acute myeloblastic leukemia—is a cancer of the myeloid origin blood cells, characterized by the rapid growth of abnormal meyloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired. In some embodiments, the disease is a solid tumor cancer. Non-limiting examples include sarcomas and carcinomas of the tissues, e.g., brain cancer, renal cancer, breast cancer, adenocarcinoma, neurological cancer, lung cancer, colorectal cancer or glioblastoma (GBM). In some embodiments, the disease is selected from breast cancer, melanoma, carcinoid, ovarian cancer, cervical cancer, pancreatic cancer, colorectal cancer, prostate cancer, endometrial cancer, renal caner, glioma, neurological cancer, skin cancer, head and neck cancer, stomach cancer, liver cancer, testis cancer, lung cancer, thyroid cancer, lymphoma, urothelial cancer, or any other cancer as identified in www.proteinatlas.org/ENSG00000122025-FLT3/pathology or Sung Hee Lim et al. Oncotarget. 2017 Jan. 10; 8(2): 3237-3245. In some embodiments, the disease is myelodysplastic syndromes. Accordingly, it would be understood by one of skill in the art that the term cancer as recited in the methods and other disclosure herein may be replaced by the term disease or myelodysplastic syndromes.

The term “suffering” as it related to the term “treatment” refers to a patient or individual who has been diagnosed with or is predisposed to a disease as disclosed herein. This patient has not yet developed characteristic disease pathology.

As used herein, a “cancer” is a disease state characterized by the presence in a subject of cells demonstrating abnormal uncontrolled replication and in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen.

The terms or “acceptable,” “effective,” or “sufficient” when used to describe the selection of any components, ranges, dose forms, etc. as disclosed herein intend that said component, range, dose form, etc. is suitable for the disclosed purpose.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped. The methods and treatments as described herein can be provides as a first line, second line, third line, fourth line or fifth line therapy.

### Modes for Carrying Out the Disclosure

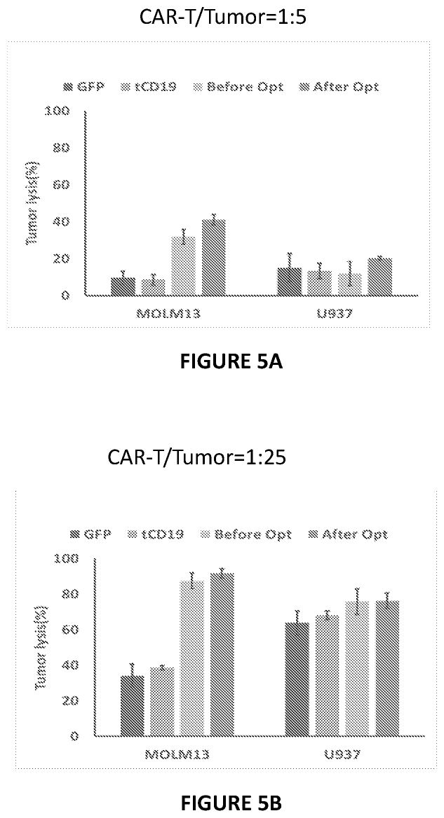

In this disclosure the generation and anti-tumor efficacy of CAR with an improved FLT3-CAR coding sequence are described. The improved sequence, which is also referred to herein as an optimized sequence, provides higher expression level of the CAR as well as the truncated protein marker on a cell, and thus is especially suitable for use in transducing a cell and identifying or isolating the transduced cell. See, e.g., FIG. 4C v.s. FIG. 4D. In further embodiments, the CAR expressing cells transduced with the optimized sequence demonstrates higher cytotoxicity against tumor cells. See, e.g., FIG. 5A.

In one aspect, provided is a nucleic acid molecule encoding an anti-FMS-like tyrosine kinase 3 (FLT3) chimeric antigen receptor (CAR). The nucleic acid molecule comprises, or alternatively consists essentially of, or yet further consists of the polynucleotide of nucleotide (nt) 55 to nt 777, or nt 1 to nt 777, or the full length of SEQ ID NO: 1 or an equivalent thereof.

In one aspect, provided is a nucleic acid molecule encoding an anti-FMS-like tyrosine kinase 3 (FLT3) antigen binding fragment comprising, or consisting essentially of, or yet further consisting of the polynucleotide of nucleotide (nt) 55 to nt 777 of SEQ ID NO:1 or an equivalent there of or the polynucleotide of nt 55 to nt 777 of SEQ ID NO: 12. In some embodiments, the nucleic acid molecule comprises, or consists essentially of, or yet further consists of the polynucleotide of nt 1 to nt 777 of SEQ ID NO: 1 or an equivalent thereof which is at least 85% identical to nt 1 to nt 777 of SEQ ID NO: 1 or comprising the polynucleotide of nt 1 to nt 777 of SEQ ID NO: 12.

In some embodiments, the equivalent is at least about 75% (including but not limited to at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%) identical to nucleotide (nt) 55 to nt 777, or nt 1 to nt 777, or the full length of SEQ ID NO: 1 with the proviso that in one aspect, the at least one of optimized nucleotides of SEQ ID NO: 1 are retained in the equivalent. Additionally or alternatively, the equivalent comprises the polynucleotide of nucleotide (nt) 55 to nt 777, or nt 1 to nt 777, or the full length of ATGGGgTGGtcaagcATtATtCTgTTtcTGGTcGCtACcGCTACAGGcGTCCAtCAGGTCCA gCTGCAGCAGCCcGGaGCcGAaCTgGTGAAGCCcGGcGCcTCccTGAAGCTGTCtTGCA AGagcagCGGcTACACaTTCACCtcCTAtTGGATGCACTGGGTGcGGCAGcGGCCcGGcC AcGGCCTgGAGTGGATCGGcGAGATcGAcCCcTCTGAtAGcTAcAAgGACTAtAAcCA GAAGTTtAAGGAtAAGGCCACAcTGACcGTGGACcGgTCtagCAAtACAGCCTACATG CACCTgtcCtctCTGACcTCcGAcGAtTCTGCcGTgTAcTATTGcGCcAGgGCcATcACcACa ACCCCtTTcGAtTTtTGGGGCCAgGGCACaACcCTgACcGTgagCagcGGaGGaGGaGGcag cGGaGGaGGaGGCTCCGGCGGcGGCGGcTCTGAcATcGTGCTgACcCAGTCcCCAGCC ACaCTGagcGTGACcCCtGGcGActcCGTgtcTCTgagCTGtcGGGCCtcCCAGtcTATcAGCA ACAAtCTgCACTGGTATCAgCAgAAgagcCAcGAGTCcCCtAGGCTgCTgATCAAGTAT GCcTCCCAaTCtATCagcGGcATCCCaagCcGcTTCtccGGCtcTGGcagcGGcACAGAcTTC ACcCTgtcTATCAACAGcGTGGAGACaGAgGAcTTcGGcGTGTATTTtTGTCAgCAGtcT AAtACaTGGCCaTAtACaTTCGGAGGaGGaACtAAaCTGGAAATcAAACGactcgagcccaaa tcttgtgacaaaactcacacatgcccaccgtgcccggatcccaaaggtaccttttgggtgctggtggtggttggtggagtcctggcttgc tatagcttgctagtaacagtggcctttattattttctgggtgaggagtaagaggagcaggctcctgcacagtgactacatgaacatgactc cccgccgccccgggcccacccgcaagcattaccagccctatgccccaccacgcgacttcgcagcctatcgctccagagtgaagttc agcaggagcgcagacgcccccgcgtaccagcagggccagaaccagctctataacgagctcaatctaggacgaagagaggagtac gatgttttggacaagagacgtggccgggaccctgagatggggggaaagccgagaaggaagaaccctcaggaaggcctgtacaatg aactgcagaaagataagatggcggaggcctacagtgagattgggatgaaaggcgagcgccggaggggcaaggggcacgatggc ctttaccagggtctcagtacagccaccaaggacacctacgacgcccttcacatgcaggccctgccccctcgc, wherein the small letter indicates any nucleotide (SEQ ID NO: 12). In some embodiments, the small letter indicates a nucleotide located in the corresponding position in SEQ ID NO: 1. In some embodiments, the equivalent does not comprises the polynucleotide of nucleotide (nt) 55 to nt 777, or nt 1 to nt 777 of

(SEQ ID NO: 14)
ATGGGATGGAGCTCTATCATCCTCTTCTTGGTAGC
AACAGCTACAGGTGTCCACCAGGTCCAACTGCAGC
AGCCTGGGGCTGAGCTTGTGAAGCCTGGGGCTTCA
TTGAAGCTGTCCTGCAAGTCTTCCGGGTACACCTT
CACCAGCTACTGGATGCACTGGGTGAGGCAGAGGC
CTGGACATGGCCTTGAGTGGATCGGAGAGATTGAT
CCTTCTGACAGTTATAAAGACTACAATCAGAAGTT
CAAGGACAAGGCCACATTGACTGTGGACAGATCCT
CCAACACAGCCTACATGCACCTCAGCAGCCTGACA
TCTGATGACTCTGCGGTCTATTATTGTGCAAGAGC
GATTACGACGACCCCCTTTGACTTCTGGGGCCAAG
GCACCACTCTCACAGTCTCCTCAGGCGGTGGCGGT
TCTGGTGGCGGTGGCTCCGGCGGTGGCGGTTCTGA
TATTGTGCTAACTCAGTCTCCAGCCACCCTGTCTG
TGACTCCAGGAGATAGCGTCAGTCTTTCCTGCAGG
GCCAGCCAGAGTATTAGCAACAACCTACACTGGTA
TCAACAAAAATCACATGAGTCTCCAAGGCTTCTCA
TCAAGTATGCTTCCCAGTCCATCTCTGGGATCCCC
TCCAGGTTCAGTGGCAGTGGATCAGGGACAGATTT
CACTCTCAGTATCAACAGTGTGGAGACTGAAGATT
TTGGAGTGTATTTCTGTCAACAGAGTAACACCTGG
CCGTACACGTTCGGAGGGGGGACCAAGCTGGAAAT
AAAACGG.

In some embodiments, the nucleic acid molecule or the equivalent thereof comprises, or alternatively consists essentially of, or yet further consists of: a polynucleotide encoding a light chain complementarity-determining region 1 (CDRL1) as set forth in RASQSISNNLH (SEQ ID NO: 15), a polynucleotide encoding a light chain complementarity-determining region 2 (CDRL2) as set forth in YASQSIS (SEQ ID NO: 16), a polynucleotide encoding a light chain complementarity-determining region 3 (CDRL3) as set forth in QQSNTWPYT (SEQ ID NO: 17), a polynucleotide encoding a heavy chain complementarity-determining region 1 (CDRH1) as set forth in SYWMH (SEQ ID NO: 18), a polynucleotide encoding a heavy chain complementarity-determining region 1 (CDRH2) as set forth in EIDPSDSYKDYNQKFKD (SEQ ID NO: 19), and a polynucleotide encoding a heavy chain complementarity-determining region 1 (CDRH3) as set forth in AITTTPFDF (SEQ ID NO: 20).

In some embodiments, the nucleic acid molecule or the equivalent thereof comprises a polynucleotide encoding an antibody heavy chain variable region of QVQLQQPGAELVKPGASLKLSCKSSGYTFTSYWMHWVRQRPGHGLEWIGEIDPSDS YKDYNQKFKDKATLTVDRSSNTAYMHLSSLTSDDSAVYYCARAITTTPFDFWGQGT TLTVSS (SEQ ID NO: 21) and an antibody light chain variable region of

(SEQ ID NO: 22)
DIVLTQSPATLSVTPGDSVSLSCRASQSISNNL
HWYQQKSHESPRLLIKYASQSISGIPSRFSGSG
SGTDFTLSINSVETEDFGVYFCQQSNTWPYTFG
GGTKLEIKR.

In some embodiments, the nucleic acid molecule or the equivalent encodes a single chain Fv (scFv) specifically recognizing and binding FLT3, a signal sequence, a spacer, a transmembrane domain, a co-stimulatory domain, and a intracellular signaling domain. In some embodiments, the nucleic acid molecule or the equivalent comprises, or consists essentially of, or yet further consists of a polypeptide that encodes a signal peptide, a polypeptide that encodes an FLT3 antigen binding fragment, a polypeptide that encodes a hinge domain, a polypeptide that encodes a transmembrane domain, a polypeptide that encodes a costimulatory signaling region, a polypeptide that encodes an intracellular signaling domain. In some embodiments, the nucleic acid molecule or the equivalent comprises, or consists essentially of, or yet further consists of a polypeptide that encodes a signal peptide, a polypeptide that encodes an FLT3 antigen binding fragment, a polypeptide that encodes a hinge domain, a polypeptide that encodes a CD28 transmembrane domain, a polypeptide that encodes a CD28 costimulatory signaling region, a polypeptide that encodes an intracellular signaling domain.

In some embodiments, the nucleic acid molecule or the equivalent as disclosed herein encodes the polypeptide of amino acid (aa) 19 to aa 259 of SEQ ID NO: 2. In further embodiments, the nucleic acid molecule or the equivalent as disclosed herein encodes the polypeptide of aa 1 to aa 259 of SEQ ID NO: 2. In some embodiments, the nucleic acid molecule or the equivalent encodes the polypeptide of SEQ ID NO: 2.

In some embodiments, the nucleic acid molecule further comprises a polynucleotide encoding a truncated CD19, or a truncated EGFR. In further embodiments, the truncated EGFR comprises, or alternatively consists essentially of, or yet further consists of the polypeptide of SEQ ID NO: 10. In yet further embodiments, the truncated CD19 comprises, or alternatively consists essentially of, or yet further consists of the polypeptide of SEQ ID NO: 13 or 11. In some embodiments, the nucleic acid molecule as disclosed herein further comprises a polynucleotide as set forth in SEQ ID NO: 28 or an equivalent thereof encoding a truncated CD19.

In some embodiments, the nucleic acid molecule further comprises a polynucleotide encoding a ribosomal skip sequence located between the polynucleotide encoding the CAR and the polynucleotide encoding the truncated CD19 or the truncated EGFR. In some embodiments, the nucleic acid molecule further comprises a T2A skip sequence and a sequence encoding the truncated EGFR or the truncated CD19. In some embodiments, the nucleic acid molecule further comprises a T2A skip sequence and a sequence encoding a truncated EGFR. In some embodiments, the nucleic acid molecule further comprises a T2A skip sequence and a sequence encoding a truncated CD19. In some embodiments, the nucleic acid molecule further comprises a T2A skip sequence and a sequence encoding a truncated EGFR.

In some embodiments, some embodiments, the nucleic acid molecule as disclosed herein comprises, or alternatively consists essentially of, or yet further consists of an equivalent of SEQ ID NO: 1. In some embodiments, the equivalent of SEQ ID NO: 1 consists of about 55% to about 60% of G and C residues, or any percentage or range there between. In some embodiments, the equivalent of SEQ ID NO: 1 consists of about 58% of G and C residues.

In some embodiments, at least about 70% of the codon(s) encoding an alanine (Ala) amino acid residue in the equivalent of SEQ ID NO: 1 consists of GCC. In some embodiments, at least about 60% of the codon(s) encoding a cysteine (Cys) amino acid residue in the equivalent of SEQ ID NO: 1 consists of TGC. In some embodiments, at least about 60% of the codon(s) encoding an aspartic acid (Asp) amino acid residue in the equivalent of SEQ ID NO: 1 consists of GAC. In some embodiments, at least about 75% of the codon(s) encoding a glutamic acid (Glu) amino acid residue in the equivalent of SEQ ID NO: 1 consists of GAG. In some embodiments, at least about 60% of the codon(s) encoding a phenylalanine (Phe) amino acid residue in the equivalent of SEQ ID NO: 1 consists of TTC. In some embodiments, at least about 55% of the codon(s) encoding a glycine (Gly) amino acid residue in the equivalent of SEQ ID NO: 1 consists of GGC. In some embodiments, at least about 25% of the codon(s) encoding a glycine (Gly) amino acid residue in the equivalent of SEQ ID NO: 1 consists of GGA. In some embodiments, at least about 80% of the codon(s) encoding a histidine (His) amino acid residue in the equivalent of SEQ ID NO: 1 consists of CAC. In some embodiments, at least about 65% of the codon(s) encoding an isoleucine (Ile) amino acid residue in the equivalent of SEQ ID NO: 1 consists of ATC. In some embodiments, at least about 70% of the codon(s) encoding a lysine (Lys) amino acid residue in the equivalent of SEQ ID NO: 1 consists of AAG. In some embodiments, at least about 65% of the codon(s) encoding a leucine (Leu) amino acid residue in the equivalent of SEQ ID NO: 1 consists of CTG. In some embodiments, at least about 90% of the codon(s) encoding a methionine (Met) amino acid residue in the equivalent of SEQ ID NO: 1 consists of ATG. In some embodiments, at least about 55% of the codon(s) encoding an asparagine (Asn) amino acid residue in the equivalent of SEQ ID NO: 1 consists of AAC. In some embodiments, at least about 40% of the codon(s) encoding a proline (Pro) amino acid residue in the equivalent of SEQ ID NO: 1 consists of CCC. In some embodiments, at least about 95% of the codon(s) encoding a glutamine (Gln) amino acid residue in the equivalent of SEQ ID NO: 1 consists of CAG. In some embodiments, at least about 25% of the codon(s) encoding an arginine (Arg) amino acid residue in the equivalent of SEQ ID NO: 1 consists of AGG. In some embodiments, at least about 25% of the codon(s) encoding an Arg amino acid residue in the equivalent of SEQ ID NO: 1 consists of CGC. In some embodiments, at least about 40% of the codon(s) encoding a serine (Ser) amino acid residue in the equivalent of SEQ ID NO: 1 consists of AGC. In some embodiments, at least about 45% of the codon(s) encoding a threonine (Thr) amino acid residue in the equivalent of SEQ ID NO: 1 consists of ACC. In some embodiments, at least about 40% of the codon(s) encoding a Thr amino acid residue in the equivalent of SEQ ID NO: 1 consists of ACA. In some embodiments, at least about 65% of the codon(s) encoding a valine (Val) amino acid residue in the equivalent of SEQ ID NO: 1 consists of GTG. In some embodiments, at least about 90% of the codon(s) encoding a tryptophan (Trp) amino acid residue in the equivalent of SEQ ID NO: 1 consists of TGG. In some embodiments, at least about 50% of the codon(s) encoding a tyrosine (Tyr) amino acid residue in the equivalent of SEQ ID NO: 1 consists of TAC. In some embodiments, at least about 45% of the codon(s) encoding a Tyr amino acid residue in the equivalent of SEQ ID NO: 1 consists of TAT.

In some embodiments, the equivalent of SEQ ID NO: 1 comprises, or alternatively consists essentially of, or yet further consists of the codon consisting of GCG at a frequency of about 0% to about 1% optionally about 0.435%, the codon consisting of GCA at a frequency of about 0% to about 1% optionally about 0.435%, the codon consisting of GCT at a frequency of about 0% to about 1% optionally about 0.652%, the codon consisting of GCC at a frequency of about 3% or higher optionally about 3.913%; the codon consisting of TGT at a frequency of about 0% to about 1% optionally about 0.652%, the codon consisting of TGC at a frequency of about 1% or higher optionally about 1.087%; the codon consisting of GAT at a frequency of about 0% to about 2% optionally about 1.739%, the codon consisting of GAC at a frequency of about 3% or higher optionally about 3.478%; the codon consisting of GAG at a frequency of about 2% or higher optionally about 2.826%, the codon consisting of GAA at a frequency of about 0% to about 1% optionally about 0.870%; the codon consisting of TTT at a frequency of about 0% to about 2% optionally about 1.304%, the codon consisting of TTC at a frequency of about 1% or higher optionally about 1.957%; the codon consisting of GGG at a frequency of about 0% to about 2% optionally about 1.087%, the codon consisting of GGA at a frequency of about 0% to about 3% optionally about 2.826%, the codon consisting of GGT at a frequency of about 0% to about 1% optionally about 0.652%, the codon consisting of GGC at a frequency of about 5% or higher optionally about 5.652%; the codon consisting of CAT at a frequency of about 0% to about 1% optionally about 0.435%, the codon consisting of CAC at a frequency of about 1% or higher optionally about 1.957%; the codon consisting of ATA at a frequency of about 0%, the codon consisting of ATT at a frequency of about 0% to about 2% optionally about 1.087%, the codon consisting of ATC at a frequency of about 2% or higher optionally about 2.174%; the codon consisting of AAG at a frequency of about 3% or higher optionally about 3.913%, the codon consisting of AAA at a frequency of about 0% to about 2% optionally about 1.522%; the codon consisting of TTG at a frequency of about 0% to about 1% optionally about 0.435%, the codon consisting of TTA at a frequency of about 0%, the codon consisting of CTG at a frequency of about 5% or higher about 5.435%, the codon consisting of CTA at a frequency of about 0% to about 1% optionally about 0.435%, the codon consisting of CTT at a frequency of about 0% to about 1% optionally about 0.435%, the codon consisting of CTC at a frequency of about 0% to about 2% optionally about 1.087%; the codon consisting of ATG at a frequency of about 1% or higher optionally about 1.957%; the codon consisting of AAT at a frequency of about 0% to about 2% optionally about 1.087%, the codon consisting of AAC at a frequency of about 1% or higher about 1.522%; the codon consisting of CCG at a frequency of about 0% to about 1% optionally about 0.652%, the codon consisting of CCA at a frequency of about 0% to about 2% optionally about 1.304%, the codon consisting of CCT at a frequency of about 0% to about 2% optionally about 1.304%, the codon consisting of CCC at a frequency of about 2% or higher optionally about 2.609%; the codon consisting of CAG at a frequency of about 4% or higher optionally about 4.783%, the codon consisting of CAA at a frequency of about 0% to about 1% optionally about 0.217%; the codon consisting of AGG at a frequency of about 1% or higher optionally about 1.739%, the codon consisting of AGA at a frequency of about 0% to about 1% optionally about 0.870%, the codon consisting of CGG at a frequency of about 0% to about 2% optionally about 1.304%, the codon consisting of CGA at a frequency of about 0% to about 1% optionally about 0.435%, the codon consisting of CGT at a frequency of about 0% to about 1% optionally about 0.217%, the codon consisting of CGC at a frequency of about 1% or higher optionally about 1.739%; the codon consisting of AGT at a frequency of about 0% to about 1% optionally about 0.870%, the codon consisting of AGC at a frequency of about 4% or higher about 4.348%, the codon consisting of TCG at a frequency of about 0%, the codon consisting of TCA at a frequency of about 0% to about 1% optionally about 0.217%, the codon consisting of TCT at a frequency of about 0% to about 3% optionally about 2.826%, the codon consisting of TCC at a frequency of about 0% to about 3% optionally about 2.609%; the codon consisting of ACG at a frequency of about 0%, the codon consisting of ACA at a frequency of about 3% or higher optionally about 3.043%, the codon consisting of ACT at a frequency of about 0% to about 1% optionally about 0.652%, the codon consisting of ACC at a frequency of about 3% or higher optionally about 3.261%; the codon consisting of GTG at a frequency of about 3% or higher optionally about 3.478%, the codon consisting of GTA at a frequency of about 0% to about 1% optionally about 0.217%, the codon consisting of GTT at a frequency of about 0% to about 1% optionally about 0.435%, the codon consisting of GTC at a frequency of about 0% to about 1% optionally about 0.870%; the codon consisting of TGG at a frequency of about 1% or higher optionally about 1.957%; the codon consisting of TAT at a frequency of about 2% or higher optionally about 2.391%, and the codon consisting of TAC at a frequency of about 2% or higher optionally about 2.609%.

In some embodiments, the equivalent of SEQ ID NO: 1 comprises a codon frequency or codon usage preference as disclosed in the Table below.

| AmAcid | Codon | Number | /1000 | Fraction |
|---|---|---|---|---|
| Ala | GCG | 2.00 | 4.35 | 0.08 |
| Ala | GCA | 2.00 | 4.35 | 0.08 |
| Ala | GCT | 3.00 | 6.52 | 0.12 |
| Ala | GCC | 18.00 | 39.13 | 0.72 |
| Cys | TGT | 3.00 | 6.52 | 0.38 |
| Cys | TGC | 5.00 | 10.87 | 0.63 |
| Asp | GAT | 8.00 | 17.39 | 0.33 |
| Asp | GAC | 16.00 | 34.78 | 0.67 |
| Glu | GAG | 13.00 | 28.26 | 0.76 |
| Glu | GAA | 4.00 | 8.70 | 0.24 |
| Phe | TTT | 6.00 | 13.04 | 0.40 |
| Phe | TTC | 9.00 | 19.57 | 0.60 |
| Gly | GGG | 5.00 | 10.87 | 0.11 |
| Gly | GGA | 13.00 | 28.26 | 0.28 |
| Gly | GGT | 3.00 | 6.52 | 0.06 |
| Gly | GGC | 26.00 | 56.52 | 0.55 |
| His | CAT | 2.00 | 4.35 | 0.18 |
| His | CAC | 9.00 | 19.57 | 0.82 |
| Ile | ATA | 0.00 | 0.00 | 0.00 |
| Ile | ATT | 5.00 | 10.87 | 0.33 |
| Ile | ATC | 10.00 | 21.74 | 0.67 |
| Lys | AAG | 18.00 | 39.13 | 0.72 |
| Lys | AAA | 7.00 | 15.22 | 0.28 |
| Leu | TTG | 2.00 | 4.35 | 0.06 |
| Leu | TTA | 0.00 | 0.00 | 0.00 |
| Leu | CTG | 25.00 | 54.35 | 0.69 |
| Leu | CTA | 2.00 | 4.35 | 0.06 |
| Leu | CTT | 2.00 | 4.35 | 0.06 |
| Leu | CTC | 5.00 | 10.87 | 0.14 |
| Met | ATG | 9.00 | 19.57 | 1.00 |
| Asn | AAT | 5.00 | 10.87 | 0.42 |
| Asn | AAC | 7.00 | 15.22 | 0.58 |
| Pro | CCG | 3.00 | 6.52 | 0.11 |
| Pro | CCA | 6.00 | 13.04 | 0.22 |
| Pro | CCT | 6.00 | 13.04 | 0.22 |
| Pro | CCC | 12.00 | 26.09 | 0.44 |
| Gln | CAG | 22.00 | 47.83 | 0.96 |
| Gln | CAA | 1.00 | 2.17 | 0.04 |
| Arg | AGG | 8.00 | 17.39 | 0.28 |
| Arg | AGA | 4.00 | 8.70 | 0.14 |
| Arg | CGG | 6.00 | 13.04 | 0.21 |
| Arg | CGA | 2.00 | 4.35 | 0.07 |
| Arg | CGT | 1.00 | 2.17 | 0.03 |
| Arg | CGC | 8.00 | 17.39 | 0.28 |
| Ser | AGT | 4.00 | 8.70 | 0.08 |
| Ser | AGC | 20.00 | 43.48 | 0.40 |
| Ser | TCG | 0.00 | 0.00 | 0.00 |
| Ser | TCA | 1.00 | 2.17 | 0.02 |
| Ser | TCT | 13.00 | 28.26 | 0.26 |
| Ser | TCC | 12.00 | 26.09 | 0.24 |
| Thr | ACG | 0.00 | 0.00 | 0.00 |
| Thr | ACA | 14.00 | 30.43 | 0.44 |
| Thr | ACT | 3.00 | 6.52 | 0.09 |
| Thr | ACC | 15.00 | 32.61 | 0.47 |
| Val | GTG | 16.00 | 34.78 | 0.70 |
| Val | GTA | 1.00 | 2.17 | 0.04 |
| Val | GTT | 2.00 | 4.35 | 0.09 |
| Val | GTC | 4.00 | 8.70 | 0.17 |
| Trp | TGG | 9.00 | 19.57 | 1.00 |
| Tyr | TAT | 11.00 | 23.91 | 0.48 |
| Tyr | TAC | 12.00 | 26.09 | 0.52 |
| End | TGA | 0.00 | 0.00 | 0.00 |
| End | TAG | 0.00 | 0.00 | 0.00 |
| End | TAA | 0.00 | 0.00 | 0.00 |

In some embodiments, the nucleic acid molecule as disclosed herein comprises, or alternatively consists essentially of, or yet further consists of an equivalent of nt 1 to nt 777 of SEQ ID NO: 1. In some embodiments, the equivalent of nt 1 to nt 777 of SEQ ID NO: 1 consists of about 55% to about 60% of G and C residues, or any percentage or range there between. In some embodiments, the equivalent of nt 1 to nt 777 of SEQ ID NO: 1 consists of about 58% of G and C residues.

In some embodiments, at least about 80% of the codon(s) encoding an alanine (Ala) amino acid residue in the equivalent of nt 1 to nt 777 of SEQ ID NO: 1 consists of GCC. In some embodiments, at least about 50% of the codon(s) encoding a cysteine (Cys) amino acid residue in the equivalent of nt 1 to nt 777 of SEQ ID NO: 1 consists of TGC. In some embodiments, at least about 50% of the codon(s) encoding a cysteine (Cys) amino acid residue in the equivalent of nt 1 to nt 777 of SEQ ID NO: 1 consists of TGT. In some embodiments, at least about 60% of the codon(s) encoding an aspartic acid (Asp) amino acid residue in the equivalent of nt 1 to nt 777 of SEQ ID NO: 1 consists of GAC. In some embodiments, at least about 70% of the codon(s) encoding a glutamic acid (Glu) amino acid residue in the equivalent of nt 1 to nt 777 of SEQ ID NO: 1 consists of GAG. In some embodiments, at least about 55% of the codon(s) encoding a phenylalanine (Phe) amino acid residue in the equivalent of nt 1 to nt 777 of SEQ ID NO: 1 consists of TTC. In some embodiments, at least about 60% of the codon(s) encoding a glycine (Gly) amino acid residue in the equivalent of nt 1 to nt 777 of SEQ ID NO: 1 consists of GGC. In some embodiments, at least about 30% of the codon(s) encoding a glycine (Gly) amino acid residue in the equivalent of nt 1 to nt 777 of SEQ ID NO: 1 consists of GGA. In some embodiments, at least about 80% of the codon(s) encoding a histidine (His) amino acid residue in the equivalent of nt 1 to nt 777 of SEQ ID NO: 1 consists of CAC. In some embodiments, at least about 80% of the codon(s) encoding an isoleucine (Ile) amino acid residue in the equivalent of nt 1 to nt 777 of SEQ ID NO: 1 consists of ATC. In some embodiments, at least about 80% of the codon(s) encoding a lysine (Lys) amino acid residue in the equivalent of nt 1 to nt 777 of SEQ ID NO: 1 consists of AAG. In some embodiments, at least about 90% of the codon(s) encoding a leucine (Leu) amino acid residue in the equivalent of nt 1 to nt 777 of SEQ ID NO: 1 consists of CTG. In some embodiments, at least about 90% of the codon(s) encoding a methionine (Met) amino acid residue in the equivalent of nt 1 to nt 777 of SEQ ID NO: 1 consists of ATG. In some embodiments, at least about 50% of the codon(s) encoding an asparagine (Asn) amino acid residue in the equivalent of nt 1 to nt 777 of SEQ ID NO: 1 consists of AAC. In some embodiments, at least about 50% of the codon(s) encoding an asparagine (Asn) amino acid residue in the equivalent of nt 1 to nt 777 of SEQ ID NO: 1 consists of AAT. In some embodiments, at least about 40% of the codon(s) encoding a proline (Pro) amino acid residue in the equivalent of nt 1 to nt 777 of SEQ ID NO: 1 consists of CCC. In some embodiments, at least about 90% of the codon(s) encoding a glutamine (Gln) amino acid residue in the equivalent of nt 1 to nt 777 of SEQ ID NO: 1 consists of CAG. In some embodiments, at least about 50% of the codon(s) encoding an arginine (Arg) amino acid residue in the equivalent of nt 1 to nt 777 of SEQ ID NO: 1 consists of CGG. In some embodiments, at least about 20% of the codon(s) encoding an Arg amino acid residue in the equivalent of nt 1 to nt 777 of SEQ ID NO: 1 consists of AGG. In some embodiments, at least about 60% of the codon(s) encoding a serine (Ser) amino acid residue in the equivalent of nt 1 to nt 777 of SEQ ID NO: 1 consists of AGC. In some embodiments, at least about 45% of the codon(s) encoding a threonine (Thr) amino acid residue in the equivalent of nt 1 to nt 777 of SEQ ID NO: 1 consists of ACC. In some embodiments, at least about 45% of the codon(s) encoding a Thr amino acid residue in the equivalent of nt 1 to nt 777 of SEQ ID NO: 1 consists of ACA. In some embodiments, at least about 70% of the codon(s) encoding a valine (Val) amino acid residue in the equivalent of nt 1 to nt 777 of SEQ ID NO: 1 consists of GTG. In some embodiments, at least about 90% of the codon(s) encoding a tryptophan (Trp) amino acid residue in the equivalent of nt 1 to nt 777 of SEQ ID NO: 1 consists of TGG. In some embodiments, at least about 60% of the codon(s) encoding a tyrosine (Tyr) amino acid residue in the equivalent of nt 1 to nt 777 of SEQ ID NO: 1 consists of TAT. In some embodiments, at least about 30% of the codon(s) encoding a Tyr amino acid residue in the equivalent of nt 1 to nt 777 of SEQ ID NO: 1 consists of TAT.

In some embodiments, the equivalent of nt 1 to nt 777 of SEQ ID NO: 1 comprises, or alternatively consists essentially of, or yet further consists of the codon consisting of GCG at a frequency of about 0% to about 1% optionally about 0.000%, the codon consisting of GCA at a frequency of about 0% to about 1% optionally about 0.000%, the codon consisting of GCT at a frequency of about 0% to about 1% optionally about 0.772%, the codon consisting of GCC at a frequency of about 3% or higher optionally about 3.861%; the codon consisting of TGT or TGC at a frequency of about 0% to about 1% optionally about 0.772%, the codon consisting of TGT or TGC at a frequency of about 0.5% or higher optionally about 0.772%; the codon consisting of GAT at a frequency of about 0% to about 2% optionally about 1.544%, the codon consisting of GAC at a frequency of about 3% or higher optionally about 3.089%; the codon consisting of GAG at a frequency of about 1% or higher optionally about 1.931%, the codon consisting of GAA at a frequency of about 0% to about 1% optionally about 0.772%; the codon consisting of TTT at a frequency of about 0% to about 2% optionally about 1.544%, the codon consisting of TTC at a frequency of about 2% or higher optionally about 2.317%; the codon consisting of GGG at a frequency of about 0% to about 1% optionally about 0.386%, the codon consisting of GGA at a frequency of about 0% to about 4% optionally about 3.861%, the codon consisting of GGT at a frequency of about 0% to about 1% optionally about 0.000%, the codon consisting of GGC at a frequency of about 7% or higher optionally about 7.722%; the codon consisting of CAT at a frequency of about 0% to about 1% optionally about 0.386%, the codon consisting of CAC at a frequency of about 1% or higher optionally about 1.931%; the codon consisting of ATA at a frequency of about 0% to about 1% optionally about 0%, the codon consisting of ATT at a frequency of about 0% to about 1% optionally about 0.772%, the codon consisting of ATC at a frequency of about 3% or higher optionally about 3.861%; the codon consisting of AAG at a frequency of about 3% or higher optionally about 3.475%, the codon consisting of AAA at a frequency of about 0% to about 1% optionally about 0.772%; the codon consisting of TTG at a frequency of about 0% to about 1% optionally about 0.000%, the codon consisting of TTA at a frequency of about 0% to about 1% optionally about 0.000%, the codon consisting of CTG at a frequency of about 7% or higher about 7.336%, the codon consisting of CTA at a frequency of about 0% to about 1% optionally about 0.000%, the codon consisting of CTT at a frequency of about 0% to about 1% optionally about 0.000%, the codon consisting of CTC at a frequency of about 0% to about 1% optionally about 0.000%; the codon consisting of ATG at a frequency of about 1% or higher optionally about 1.158%; the codon consisting of AAC or AAT at a frequency of about 0% to about 2% optionally about 1.158%, the codon consisting of AAC or AAT at a frequency of about 1% or higher about 1.158%; the codon consisting of CCG at a frequency of about 0% to about 1% optionally about 0.000%, the codon consisting of CCA at a frequency of about 0% to about 2% optionally about 1.158%, the codon consisting of CCT at a frequency of about 0% to about 2% optionally about 1.158%, the codon consisting of CCC at a frequency of about 1% or higher optionally about 1.544%; the codon consisting of CAG at a frequency of about 5% or higher optionally about 5.019%, the codon consisting of CAA at a frequency of about 0% to about 1% optionally about 0.386%; the codon consisting of AGG at a frequency of about 0% to about 1% optionally about 0.772%, the codon consisting of AGA at a frequency of about 0% to about 1% optionally about 0.000%, the codon consisting of CGG at a frequency of about 1% or higher optionally about 1.544%, the codon consisting of CGA at a frequency of about 0% to about 1% optionally about 0.386%, the codon consisting of CGT at a frequency of about 0% to about 1% optionally about 0.000%, the codon consisting of CGC at a frequency of about 0% to about 1% optionally about 0.386%; the codon consisting of AGT at a frequency of about 0% to about 1% optionally about 0.000%, the codon consisting of AGC at a frequency of about 6% or higher about 6.178%, the codon consisting of TCG at a frequency of about 0% to about 1% optionally about 0.000%, the codon consisting of TCA at a frequency of about 0% to about 1% optionally about 0.386%, the codon consisting of TCT at a frequency of about 4% or higher optionally about 4.633%, the codon consisting of TCC at a frequency of about 4% or higher optionally about 4.247%; the codon consisting of ACG at a frequency of about 0% to about 1% optionally about 0.000%, the codon consisting of ACA at a frequency of about 4% or higher optionally about 4.247%, the codon consisting of ACT at a frequency of about 0% to about 1% optionally about 0.386%, the codon consisting of ACC at a frequency of about 4% or higher optionally about 4.247%; the codon consisting of GTG at a frequency of about 3% or higher optionally about 3.861%, the codon consisting of GTA at a frequency of about 0% to about 1% optionally about 0.386%, the codon consisting of GTT at a frequency of about 0% to about 1% optionally about 0.000%, the codon consisting of GTC at a frequency of about 0% to about 2% optionally about 1.158%; the codon consisting of TGG at a frequency of about 2% or higher optionally about 2.703%; the codon consisting of TAT at a frequency of about 2% or higher optionally about 2.703%, and the codon consisting of TAC at a frequency of about 0% to about 2% optionally about 1.544%.

In some embodiments, the equivalent of nt 1 to nt 777 of SEQ ID NO: 1 comprises a codon frequency or codon usage preference as disclosed in the Table below.

| AmAcid | Codon | Number | /1000 | Fraction |
|---|---|---|---|---|
| Ala | GCG | 0.00 | 0.00 | 0.00 |
| Ala | GCA | 0.00 | 0.00 | 0.00 |
| Ala | GCT | 2.00 | 7.72 | 0.17 |
| Ala | GCC | 10.00 | 38.61 | 0.83 |
| Cys | TGT | 2.00 | 7.72 | 0.50 |
| Cys | TGC | 2.00 | 7.72 | 0.50 |
| Asp | GAT | 4.00 | 15.44 | 0.33 |
| Asp | GAC | 8.00 | 30.89 | 0.67 |
| Glu | GAG | 5.00 | 19.31 | 0.71 |
| Glu | GAA | 2.00 | 7.72 | 0.29 |
| Phe | TTT | 4.00 | 15.44 | 0.40 |
| Phe | TTC | 6.00 | 23.17 | 0.60 |
| Gly | GGG | 1.00 | 3.86 | 0.03 |
| Gly | GGA | 10.00 | 38.61 | 0.32 |
| Gly | GGT | 0.00 | 0.00 | 0.00 |
| Gly | GGC | 20.00 | 77.22 | 0.65 |
| His | CAT | 1.00 | 3.86 | 0.17 |
| His | CAC | 5.00 | 19.31 | 0.83 |
| Ile | ATA | 0.00 | 0.00 | 0.00 |
| Ile | ATT | 2.00 | 7.72 | 0.17 |
| Ile | ATC | 10.00 | 38.61 | 0.83 |
| Lys | AAG | 9.00 | 34.75 | 0.82 |
| Lys | AAA | 2.00 | 7.72 | 0.18 |
| Leu | TTG | 0.00 | 0.00 | 0.00 |
| Leu | TTA | 0.00 | 0.00 | 0.00 |
| Leu | CTG | 19.00 | 73.36 | 1.00 |
| Leu | CTA | 0.00 | 0.00 | 0.00 |
| Leu | CTT | 0.00 | 0.00 | 0.00 |
| Leu | CTC | 0.00 | 0.00 | 0.00 |
| Met | ATG | 3.00 | 11.58 | 1.00 |
| Asn | AAT | 3.00 | 11.58 | 0.50 |
| Asn | AAC | 3.00 | 11.58 | 0.50 |
| Pro | CCG | 0.00 | 0.00 | 0.00 |
| Pro | CCA | 3.00 | 11.58 | 0.30 |
| Pro | CCT | 3.00 | 11.58 | 0.30 |
| Pro | CCC | 4.00 | 15.44 | 0.40 |
| Gln | CAG | 13.00 | 50.19 | 0.93 |
| Gln | CAA | 1.00 | 3.86 | 0.07 |
| Arg | AGG | 2.00 | 7.72 | 0.25 |
| Arg | AGA | 0.00 | 0.00 | 0.00 |
| Arg | CGC | 4.00 | 15.44 | 0.50 |
| Arg | CGA | 1.00 | 3.86 | 0.13 |
| Arg | CGT | 0.00 | 0.00 | 0.00 |
| Arg | CGC | 1.00 | 3.86 | 0.13 |
| Ser | AGT | 0.00 | 0.00 | 0.00 |
| Ser | AGC | 16.00 | 61.78 | 0.40 |
| Ser | TCG | 0.00 | 0.00 | 0.00 |
| Ser | TCA | 1.00 | 3.86 | 0.03 |
| Ser | TCT | 12.00 | 46.33 | 0.30 |
| Ser | TCC | 11.00 | 42.47 | 0.28 |
| Thr | ACG | 0.00 | 0.00 | 0.00 |
| Thr | ACA | 11.00 | 42.47 | 0.48 |
| Thr | ACT | 1.00 | 3.86 | 0.04 |
| Thr | ACC | 11.00 | 42.47 | 0.48 |
| Val | GTG | 10.00 | 38.61 | 0.77 |
| Val | GTA | 0.00 | 0.00 | 0.00 |
| Val | GTT | 0.00 | 0.00 | 0.00 |
| Val | GTC | 3.00 | 11.58 | 0.23 |
| Trp | TGG | 7.00 | 27.03 | 1.00 |
| Tyr | TAT | 7.00 | 27.03 | 0.64 |
| Tyr | TAC | 4.00 | 15.44 | 0.36 |
| End | TGA | 0.00 | 0.00 | 0.00 |
| End | TAG | 0.00 | 0.00 | 0.00 |
| End | TAA | 0.00 | 0.00 | 0.00 |

some embodiments, the nucleic acid molecule as disclosed herein comprises, or alternatively consists essentially of, or yet further consists of an equivalent of nt 55 to nt 777 of SEQ ID NO: 1.

In some embodiments, the equivalent of SEQ ID NO: 1 comprises nucleotide(nt) 55 to nt 408 of SEQ ID NO: 1. In some embodiments, the equivalent of SEQ ID NO: 1 comprises nt 454 to nt 777 of SEQ ID NO: 1. In some embodiments, the equivalent of SEQ ID NO: 1 comprises nt 841 to nt 921 of SEQ ID NO: 1. In some embodiments, the equivalent of SEQ ID NO: 1 comprises nt 922 to nt 1044 of SEQ ID NO: 1. In some embodiments, the equivalent of SEQ ID NO: 1 comprises nt 1045 to nt 1380 of SEQ ID NO: 1. In some embodiments, the equivalent of SEQ ID NO: 1 comprises nt 55 to nt 408 of SEQ ID NO: 1 and nt 454 to nt 777 of SEQ ID NO: 1. In some embodiments, the equivalent of SEQ ID NO: 1 comprises nt 55 to nt 408 of SEQ ID NO: 1, nt 454 to nt 777 of SEQ ID NO: 1, nt 841 to nt 921 of SEQ ID NO: 1, nt 922 to nt 1044 of SEQ ID NO: 1, and nt 1045 to nt 1380 of SEQ ID NO: 1. In some embodiments, the nucleic acid molecule comprises, or alternatively consists essentially of, or yet further consists of SEQ ID NO: 1. In some embodiments, the equivalent of SEQ ID NO: 1 comprises, or alternatively consists essentially of, or yet further consists of the polynucleotide of SEQ ID NO: 14 comprising at least one nt residue substituted with the aligned nt residue of SEQ ID NO: 1. In further embodiments, the substituted nt residue is different from the original nt residue.

In one aspect, provided is a nucleic acid molecule that encodes the CAR. In some embodiments, the nucleic acid molecule further comprise the necessary regulatory sequences, e.g., a promoter for expression in a cell, or an enhancer. In some embodiments, the nucleic acid molecule further comprise a first regulatory sequence directing the expression of the CAR. In further embodiments, the regulatory sequences comprises one or more of the following: a promoter, an intron, an enhancer, or a polyadenylation signal. In some embodiments, the promoter is an EF1alpha promoter. In some embodiments, the promoter is a CMV promoter. In some embodiments, the promoter is a MMLV promoter. In further embodiments, the nucleic acid molecule further encode a truncated protein marker that may be regulated from the same first regulatory sequence or a second regulatory sequence, such as a promoter element. In some embodiments, the second promoter comprises an EF1 alpha promoter. As is apparent to the skilled artisan, the promoter(s) are selected for the host expression system and will vary with the host and the expression vector and intended use. In one embodiment, any one or both of the regulatory sequences can be cell specific or tissue specific.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CLINIMACS® device to immunomagnetically select cells expressing the desired receptors. The cells enriched for central memory T cells can be activated with anti-CD3/CD28, transduced with, for example, a lentiviral vector that directs the expression of the CAR as well as a non-immunogenic surface marker for in vivo detection, ablation, and potential ex vivo selection. The activated/genetically modified CAR T cells can be expanded in vitro with IL-2/IL-15 and then cryopreserved. Additional methods of preparing CAR T cells can be found in PCT/US2016/043392.

In some embodiments, further non-limiting examples of FLT3 CDR domain amino acid sequences are described in U.S. Pat. No. 10,961,312, WO2020/010284, Tables 1-4 of the US Patent Application No.: US20180346601, Table V of US Patent Application No.: US20180037657, Table 10 of US Patent Application No.: US20170037149, Table V of US Patent Application No.: US20160272716, Tables 1-3 of US Patent Application No.: US20110091470 and Tables 1-3 of US Patent Application No.: US20090297529. Non-limiting examples of FLT3 heavy chain variable region and light chain variable region amino acid sequences are described in U.S. Pat. No. 10,961,312, WO2020/010284, Tables 1 and 3 of the US Patent Application No.: US20180346601, Table X of US Patent Application No.: US20180037657, Table 10 of US Patent Application No.: US20170037149 and Table VII of US Patent Application No.: US20160272716.

In one aspect, the polynucleotide further encodes a transmembrane domain that comprises, or consists essentially of, or yet further consists of, a CD28 transmembrane domain or a CD8α transmembrane domain. In another aspect, the polynucleotide further encodes an intracellular domain that comprises, or alternatively consists essentially of, or yet further consists of, a CD28 costimulatory signaling region or a 4-1BB costimulatory signaling region or both. In a further aspect, the polynucleotide further comprises a polynucleotide that encodes a CD3 zeta signaling domain.

In some embodiments, a CAR or a cytoplasmic domain thereof as disclosed herein further comprises an IL2Rβ or a fragment thereof. In further embodiments, the fragments of IL2Rβ comprises, or alternatively consists essentially of, or yet consists of a JAK-STAT activation domain of the IL2Rβ, facilitating activation of the immune cell.

In some embodiments, the CAR expressing cell further expresses and optionally secrets an immunoregulatory molecular or a cytokine. In further embodiments, the CAR expressing cell comprises a lower expression level or a lower activity of a suppressor of the immunoeregulatory molecular or a cytokine. In one embodiment, the CAR expressing cell expresses IL-15 but does not express cytokine-inducible Src homology 2-containing protein (CIS). See, for example, Daher et al. Blood 2021 Feb. 4; 137(5):624-636.

### Truncated EGFR and Truncated CD19

In some embodiments, the CAR expressing cell may also comprise a switch mechanism for controlling expression or activation or both expression and activation of the CAR. For example, a CAR may comprise, or consist of, or consist essentially of an extracellular, transmembrane, and intracellular domain, in which the extracellular domain comprises a target-specific binding element that binds a label, binding domain, or tag that is specific for a molecule other than the target antigen that is expressed on or by a target cell (such as a cancer cell). In some embodiments, such label, binding domain or tag recognizes and binds the target antigen that is expressed on or by the target cell. In such embodiments, the specificity of the CAR is provided by a second construct that comprises, consists, or consists essentially of a target antigen binding domain and a domain on the CAR that is recognized by or binds to the label, binding domain, or tag. See, e.g., WO 2013/044225, WO 2016/000304, WO 2015/057834, WO 2015/057852, WO 2016/070061, U.S. Pat. No. 9,233,125, US 2016/0129109. In this way, a T-cell, NK cell or other cells that express the CAR can be administered to a subject, but it cannot bind a target antigen (i.e., FLT3) until a second composition comprising the label, binding domain, or tag, such as an FLT3-specific binding domain is administered.

CARs of the present disclosure may likewise require multimerization in order to active their function (see, e.g., US 2015/0368342, US 2016/0175359, US 2015/0368360) and/or an exogenous signal, such as a small molecule drug (US 2016/0166613, Yung et al., Science, 2015) in order to elicit an immune cell response, such as a T-cell response or a NK cell response.

Furthermore, the disclosed CAR expressing cell can comprise a “suicide switch” to induce cell death of the CAR expressing cells following treatment (Buddee et al., PLoS One, 2013) or to downregulate expression of the CAR following binding to the target antigen (WO 2016/011210). A non-limiting exemplary suicide switch or suicide gene is iCasp. In some embodiments, a CAR and/or a cytoplasmic domain thereof as disclosed herein further comprise a suicide gene product. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). In some embodiments, the suicide gene product is a truncated protein maker as disclosed herein.

The CD3ζ signaling domain can be followed by a ribosomal skip sequence (e.g., LEGGGEGRGSLLTCGDVEENPGPR; SEQ ID NO: 9) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVA FRGDSFTHTPPLDPQELDILKTVKEITGFLLIQAWPENRTDLHAFENLEIIRGRTKQHG QFSLAVVSLNITSLGLRSLKEISDGDVIISGNKNLCYANTINWKKLFGTSGQKTKIISN RGENSCKATGQVCHALCSPEGCWGPEPRDCVSCRNVSRGRECVDKCNLLEGEPREF VENSECIQCHPECLPQAMNITCTGRGPDNCIQCAHYIDGPHCVKTCPAGVMGENNTL VWKYADAGHVCHLCHPNCTYGCTGPGLEGCPTNGPKIPSIATGMVGALLLLLVVAL GIGLFM (SEQ ID NO: 10). In some cases, the truncated EGFR has 1, 2, 3, 4 of 5 amino acid changes (preferably conservative) compared to SEQ ID NO: 10.

Alternatively the CD3ζ signaling domain can be followed by a ribosomal skip sequence (e.g., LEGGGEGRGSLLTCGDVEENPGPR; SEQ ID NO: 9) and a truncated CD19 having a sequence that is at least 90%, at least 95%, at least 98% identical to or identical to:

(SEQ ID NO: 13)
MPPPRLLFFLLFLTPMEVRPEEPLVVKVEEGDNAV
LQCLKGTSDGPTQQLTWSRESPLKPFLKLSLGLPG
LGIHMRPLAIWLFIFNVSQQMGGFYLCQPGPPSEK
AWQPGWTVNVEGSGELFRWNVSDLGGLGCGLKNRS
SEGPSSPSGKLMSPKLYVWAKDRPEIWEGEPPCVP
PRDSLNQSLSQDLTMAPGSTLWLSCGVPPDSVSRG
PLSWTHVHPKGPKSLLSLELKDDRPARDMWVMETG
LLLPRATAQDAGKYYCHRGNLTMSFHLEITARPVL
WHWLLRTGGWKVSAVTLAYLIFCLCSLVGILHLQR
ALVLRRKR.

### Vector

This disclosure also provides a vector that comprises, or alternatively consists essentially of, or yet further consists of any one or more of the polynucleotides as disclosed herein. In some embodiments, the polynucleotide or vector further comprises, or consists essentially of, or yet further consists of, a regulatory element to drive expression of the polynucleotide or the CAR. In further embodiments, the regulatory element comprises one or more of the following: a promoter, an intron, an enhancer, or a polyadenylation signal. In some embodiments, the vector further comprises a detectable or purification marker.

In some aspects, provided is a vector comprising, or alternatively consisting essentially of, or yet further consisting of a nucleic acid molecule as disclosed herein or a complementary nucleic acid molecule thereof. In some embodiments, the vector is a viral vector. Additionally or alternatively, the vector is an expression vector. In some embodiments, the vector is a lentiviral vector. In some embodiments, the vector is a retroviral vector. In some embodiments, in the vector, the expression of the nucleotide sequence of SEQ ID NO: 1 or the equivalent thereof is under the control of a promoter, optionally an EF-1alpha promoter, a CMV promoter, or a MMLV promoter.

In some embodiments, the vector is a non-viral vector (such as a plasmid). In other embodiments, the vector is a viral vector, non-limiting examples of such are selected from the group of a retroviral vector, a lentiviral vector, an adenoviral vector, and an adeno-associated viral vector. In some embodiments, the nucleic acid molecule as disclosed herein can be inserted into a vector, such as an expression vector, e.g., a lentiviral vector or retroviral vector (between the 5′ and 3′ LTRs) or an adenovirus vector or any other vectors that can express a gene from.

In some embodiments, the vector is derived from or based on a wild-type virus. In further embodiments, the vector is derived from or based on a wild-type lentivirus. Examples of such include without limitation, human immunodeficiency virus (HIV), equine infectious anemia virus (EIAV), visna/maedi virus (VMV), caprine arthritis-encephalitis virus (CAEV), equine infectious anemia virus (EIAV), simian immunodeficiency virus (SIV), bovine immunodeficiency virus (BIV) and feline immunodeficiency virus (FIV). Alternatively, it is contemplated that other retrovirus can be used as a basis for a vector backbone such as murine leukemia virus (MLV). In some embodiments, retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charite Medical School, Institute of Virology (CBF), Berlin, Germany. Recombinant lentiviral vectors are known in the art, e.g., see U.S. Pat. Nos. 6,924,123; 7,056,699; 7,419,829 and 7,442,551, incorporated herein by reference. It will be evident that a viral vector according to the disclosure need not be confined to the components of a particular virus. The viral vector may comprise components derived from two or more different viruses, and may also comprise synthetic components. Vector components can be manipulated to obtain desired characteristics, such as target cell specificity.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome, the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

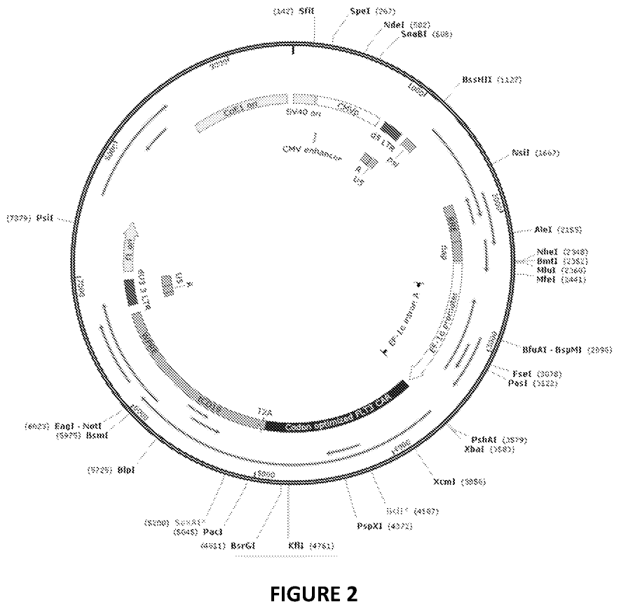

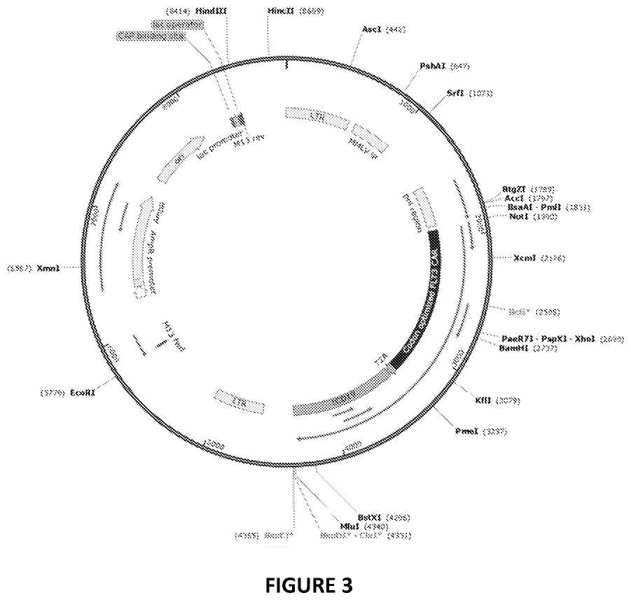

In some embodiments, provided is a vector for expression of a nucleic acid molecule as disclosed herein in a cell, i.e., an expression vector. In some embodiments, provided is a vector for producing an expression vector, i.e., a production vector or a packaging vector, such as a plasmid for duplicating a viral vector genome, and such vector genome after appropriate packaging into the viral vector is able to transduce a cell resulting in expression of the nucleic acid molecule in the cell. Two exemplified production vectors are illustrated in FIG. 2 and FIG. 3.

The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently co-transfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then co-cultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either co-transfection with a selectable marker or infection with pseudotyped virus. In both cases, the vectors integrate. Alternatively, vectors can be introduced in an episomally maintained plasmid. High titer recombinant retrovirus-containing supernatants are produced.

As is apparent, when used clinically in a human patient, marker or purification tags will be omitted from the construct. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.”

Further methods of introducing exogenous nucleic acids into the art are known and include but are not limited to gene delivery using one or more of RNA electroporation, nanotechnology, sleeping beauty vectors, retroviruses, and/or adenoviruses.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Cells

In some aspects, provided is a population of human T or NK cells comprising a nucleic acid molecule as disclosed herein or a vector as disclosed herein. In some embodiments, the isolated cell or the population of human T or NK cells comprise central memory T cells, NK cells, naive memory T cells, pan T cells, or PBMC substantially depleted for CD25+ cells and CD14+ cells.

In further aspects, provided is an isolated cell comprising a nucleic acid molecule as disclosed herein or a vector as disclosed herein. In some embodiments, the cell is selected from the group of: an immune cell, an NK cell, a T cell, a stem cell, a progenitor cell or a precursor cell. Also provided is a population of cells comprising the isolated cell.

In yet further embodiments, provided is a composition comprising, or consisting essentially of, or yet further consisting of a cell population as disclosed herein a cell as disclosed herein, and a carrier, and optionally a stabilizer, preservative or cryopreservative.

In some embodiments, the cell population is substantially homogenous. In further embodiments, the population comprises at least 90% (including but not limited to at least about 91%, at least about 92%, at least about 93%, at least about 94%, at least about 95%, at least about 96%, at least about 97%, at least about 98%, or at least about 99%) of cells of the same at least one cell subtype, e.g., at least 90% of the cells are T cells or NK cells.

Also provided herein is an isolated cell comprising, or alternatively consisting essentially of, or yet further consisting of, any one or more of: a vector as disclosed herein, a CAR as disclosed herein, a truncated protein maker as disclosed herein, or a polynucleotide as described herein.

The cell can be a prokaryotic cell (e.g., a bacterial cell) or a eukaryotic cell. Non-limiting examples of eukaryotic cells include, but are not limited to, a yeast cell, an animal cell, a mammalian cell, a bovine cell, a feline cell, a canine cell, a murine cell, an equine cell, or a human cell. In some embodiments, the isolated cell expresses the CAR. In further embodiments, the isolated cell expresses the truncated protein marker. In some embodiments, the cell further comprise a detectable or purification marker.

In some embodiments, the eukaryotic cell, mammalian or human cell is an immune cell, optionally a T-cell, a B cell, a NK cell, an NKT cell, a dendritic cell, a myeloid cell, a monocyte, a macrophage, any subsets thereof, or any other immune cell. In some embodiments, the cell is an immune cell optionally selected from a T-cell, a B cell, an NK cell, an NKT cell, a dendritic cell, a myeloid cell, a monocyte, a macrophage. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell. In certain embodiments, the isolated cell is an NKT-cell, e.g., an animal NKT-cell, a mammalian NKT-cell, a feline NKT-cell, a canine NKT-cell or a human NKT-cell. In certain embodiments, the isolated cell is a B-cell, e.g., an animal B-cell, a mammalian B-cell, a feline B-cell, a canine B-cell or a human B-cell. It is appreciated that the same or similar embodiments for each species apply with respect to dendritic cells, myeloid cells, monocytes, macrophages, any subsets of these or the T-cells, NK-cells, NKT-cells, and B-cells as described, or any other immune cells. In further embodiments, the T cell is a gamma-delta T cell. In one aspect, the cell is a T cell that has been modified to remove CD52 expression using gene editing technology, e.g., CRISPR or TALEN.

In some embodiments, the cell is selected from a Hematopoietic stem cell (HSC), an induced pluripotent stem cell (iPSCs), or an immune cell. In further embodiments, the immune cell is derived from hematopoietic stem cells (HSCs) or induced pluripotent stem cells (iPSCs).

The cells may be derived from patients, donors, or cell lines, such as those available off-the-shelf. The cells can be autologous or allogeneic to the subject being treated. In some embodiments, prior to expansion and genetic modification of the cells disclosed herein, cells may be obtained from a subject—for instance, in embodiments involving autologous therapy—or a commercially available cell line or culture, or a stem cell such as an induced pluripotent stem cell (iPSC).

Cells can be obtained from a number of sources in a subject, including peripheral blood, peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies DYNABEADS® System; STEMcell Technologies EASYSEP™ ROBOSEP™, ROSETTESEP™, SEPMATE™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells. Alternate non-limiting examples of cells that may be isolated according to known techniques include bulked T-cells, NK T-cells, and gamma delta T-cells.

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™) NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Plyi, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

In some embodiments, T cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Also provided is a cell population comprising, or alternatively consisting essentially of, or yet consisting of a cell as disclosed herein. In further embodiments, the cell population is substantially homogenous. In some embodiments, the cell population comprises, or alternatively consists essentially of, or yet consists of a Hematopoietic stem cell (HSC), an induced pluripotent stem cell (iPSCs), or an immune cell. In some embodiments, the immune cells is selected from a group consisting of T-cells, B cells, NK cells, NKT cells, dendritic cells, myeloid cells, monocytes, or macrophages. In some embodiments, the immune cell is derived from HSCs or iPSCs. In one embodiment, the cell is a T cell that has been modified to remove CD52 expression using gene editing technology, e.g., CRISPR or TALEN.

In some embodiments, provided is a method of preparing CAR T or NK cells. The method comprises, or alternatively consists essentially of, or yet further consists of providing a population of autologous or allogeneic human T or NK cells and transducing the T or NK cells by a vector as disclosed herein or a nucleic acid molecule as disclosed herein.

In some embodiments, provided is a method of preparing a CAR expressing cell. The method comprises, or alternatively consists essentially of, or yet further consists of transducing the cell with a vector as disclosed herein or a nucleic acid molecule as disclosed herein.

In some aspects, the disclosure is drawn to a method of producing a CAR-expressing cell comprising transducing an isolated cell with any of the polynucleotides as disclosed herein. In further embodiments, the method further comprises selecting and isolating the cell expressing the CAR. In certain embodiments, the method of producing a CAR expressing cell further comprises, or alternatively consists essentially of, or yet further consists of activating and expanding the population of CAR expressing cells.

Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the tumor relevant antigen ex vivo can activate and expand the selected CAR expressing cell subpopulation. Alternatively, the cells may be activated in vivo by interaction with a tumor relevant antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® System activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

### Lentiviral Vector for Expression of FLT3-Specific CAR

The FLT3-specific CAR can be expressed in T cells or NK cells using any of a variety of vectors, including lentiviral vectors and retroviral vectors.

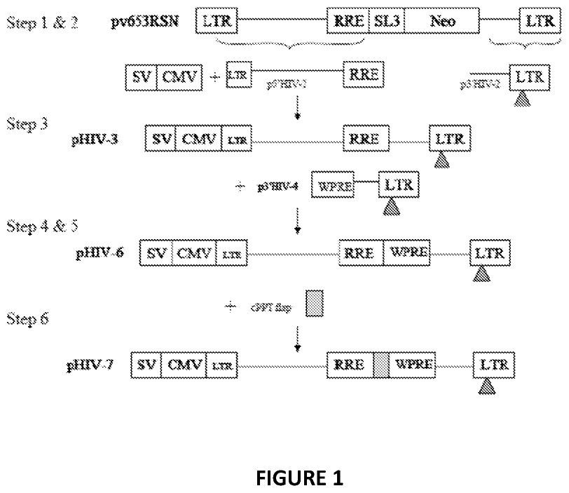

The pHIV7 vector, developed at the T Cell Therapeutics Research Laboratory (TCTRL) at City of Hope (Duarte Calif.), is a useful vector for CAR expression. This vector uses the human EF1 promoter to drive expression of the CAR. Construction of pHIV7 is schematically depicted in FIG. 1. Briefly, pv653RSN, containing 653 bp from gag-pol plus 5′ and 3′ long-terminal repeats (LTRs) with an intervening SL3-neomycin phosphotransferase gene (Neo), was subcloned into pBluescript, as follows: In Step 1, the sequences from 5′ LTR to rev-responsive element (RRE) made p5′HIV-1 51, and then the 5′ LTR was modified by removing sequences upstream of the TATA box, and ligated first to a CMV enhancer and then to the SV40 origin of replication (p5′HIV-2). In Step 2, after cloning the 3′ LTR into pBluescript to make p3′HIV-1, a 400-bp deletion in the 3′ LTR enhancer/promoter was made to remove cis-regulatory elements in HIV U3 and form p3′HIV-2. In Step 3, fragments isolated from the p5′HIV-3 and p3′HIV-2 were ligated to make pHIV-3. In Step 4, the p3′HIV-2 was further modified by removing extra upstream HIV sequences to generate p3′HIV-3 and a 600-bp BamHI-SalI fragment containing WPRE was added to p3′HIV-3 to make the p3′HIV-4. In Step 5, the pHIV-3 RRE was reduced in size by PCR and ligated to a 5′ fragment from pHIV-3 (not shown) and to the p3′HIV-4, to make pHIV-6. In Step 6, a 190-bp BglII-BamHI fragment containing the cPPT DNA flap sequence from HIV-1 pNL4-3 was amplified from pNL4-3 and placed between the RRE and the WPRE sequences in pHIV6 to make pHIV-7. This parent plasmid pHIV7-GFP (GFP, green fluorescent protein) was used to package the parent vector using a four-plasmid system.

A packaging signal, psi ψ, is required for efficient packaging of viral genome into the vector. The RRE and WPRE enhance the RNA transcript transport and expression of the transgene. The flap sequence, in combination with WPRE, has been demonstrated to enhance the transduction efficiency of lentiviral vector in mammalian cells.

The helper functions, (which are required for production of the viral vector), are divided into three separate plasmids to reduce the probability of generation of replication competent lentivirus via recombination: 1) pCgp encodes the gag/pol protein required for viral vector assembly; 2) pCMV-Rev2 encodes the Rev protein, which acts on the RRE sequence to assist in the transportation of the viral genome for efficient packaging; and 3) pCMV-G encodes the glycoprotein of the vesiculo-stomatitis virus (VSV), which is required for infectivity of the viral vector.

There is minimal DNA sequence homology between the pHIV7 encoded vector genome and the helper plasmids. The regions of homology include a packaging signal region of approximately 600 nucleotides, located in the gag/pol sequence of the pCgp helper plasmid; a CMV promoter sequence in all three helper plasmids; and a RRE sequence in the helper plasmid pCgp. It is highly improbable that replication competent recombinant virus could be generated due to the homology in these regions, as it would require multiple recombination events. Additionally, any resulting recombinants would be missing the functional LTR and tat sequences required for lentiviral replication. The CMV promoter was replaced by the EF1α-HTLV promoter (EF1p). The EF1p has 563 bp and was introduced into epHIV7 using NruI and NheI, after the CMV promoter was excised. The lentiviral genome, excluding gag/pol and rev that are necessary for the pathogenicity of the wild-type virus and are required for productive infection of target cells, has been removed from this system. In addition, the vector construct does not contain an intact 3′LTR promoter, so the resulting expressed and reverse transcribed DNA proviral genome in targeted cells will have inactive LTRs. As a result of this design, no HIV-I derived sequences will be transcribed from the provirus and only the therapeutic sequences will be expressed from their respective promoters. The removal of the LTR promoter activity in the SIN vector is expected to significantly reduce the possibility of unintentional activation of host genes.

### Compositions

Further provided herein are compositions comprising, or alternatively consisting essentially of, or yet further consisting of, a carrier and any one or more of the polynucleotides as disclosed herein, a polypeptide as disclosed herein, any one of the vectors as disclosed herein, any one of the cells as disclosed herein, or a cell population as disclosed herein. In some embodiments, the carrier is a pharmaceutically acceptable carrier.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for local or systemic administration, e.g., oral, intravenous, intracranial, topical, enteral, and/or parenteral administration. In certain embodiments, the compositions of the present disclosure are formulated for intravenous administration.

Administration of the cells or compositions can be effected in one dose, continuously or intermittently throughout the course of treatment and an effective amount to achieve the desired therapeutic benefit is provided. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In further embodiments, the cells and composition of the disclosure further comprises an agent used in a combined therapy. In yet a further embodiment, the cells and composition of the disclosure can be administered in combination with other treatments or therapy. In one aspect, a sample from the subject is isolated and assayed for FLT3 expression prior to administration of the disclosed therapy. Thus, in one aspect the methods further comprise assaying for FLT3 expression in a sample isolated from the subject, the sample containing a cancer or tumor cell.

The cells and populations of cell are administered to the host and/or subject using methods known in the art and described, for example, in PCT/US2011/064191. When the subject is a non-human animal, this administration of the cells or compositions of the disclosure can be done for therapy or to generate an animal model of the desired disease, disorder, or condition for experimental and screening assays for the therapy or combination therapies

In some aspects, the disclosure is drawn to an isolated complex comprising, or alternatively consisting essentially of, or yet consisting of any one of the isolated cells as disclosed herein bound to a cancer or tumor cell, wherein the cancer or tumor cell is bound to the CAR-expressing isolated cell by the antigen binding domain of the antigen or tumor targeting antibody expressed by the CAR.

### Methods of Use

In some embodiments, provided is a method of treating a human patient suffering from cancer, e.g. a FLT3 expressing cancer such as acute myeloid leukemia. The method comprises, or alternatively consists essentially of, or yet further consists of administering a population of autologous or allogeneic human cells, e.g., immune cells, T or NK cells, stem cells, progenitor cells, cord blood cells, hematopoietic stem cells, comprising a nucleic acid molecule as disclosed herein. The therapy can be combined with other therapies and can be administered as a first line, second line, third line, fourth line, or fifth line therapy. The cancer can be a primary cancer or metastatic or recurrent cancer. In one aspect, the patient has been treated with an agent to increase FLT3 expression on the cancer cell prior to being treated.

In some embodiments, provided is a method of treating a patient in need thereof. In one aspect, the patient is suffering from a FLT3-expressing cancer or tumor. The method comprises, or alternatively consists essentially of, or yet further consists of administering a cell population as disclosed herein to the patient. The cells, e.g., immune cells, T or NK cells, stem cells, progenitor cells, cord blood cells, hematopoietic stem cells can be allogenic or autologous to the patient being treated. The therapy can be combined with other therapies and can be administered as a first line, second line, third line, fourth line, or fifth line therapy. The cancer can be a primary cancer or metastatic or recurrent cancer. In one aspect, the patient has been treated with an agent to increase FLT3 expression on the cancer cell prior to being treated.

In some embodiments, the patient harbors tumor cell having an activating mutation in Fms-like tyrosine kinase 3 (FLT3). In further embodiments, the patient harbors tumor cell having an FLT3-ITD mutation.

In some embodiments, the chimeric antigen receptor expressing cell is administered locally or systemically.

In some embodiments, the chimeric antigen receptor expressing cell is administered by single or repeat dosing. In some embodiments, the administration is repeated for at least once, or at least twice, or more times.

In some embodiments, the cell population comprises, or alternatively consists essentially of, or yet further consists of an NK cell. In some embodiments, the NK cell is allogenic to the patient.

In some embodiments, the method further comprises administering a second cell population as disclosed herein to the patient. In further embodiments, the second cell population comprises a CAR T cell. The cells can be autologous or allogenic to the patient. In yet further embodiments, the CAR T cell is autologous to the patient.

In some embodiments, the cell population comprises, or alternatively consists essentially of, or yet further consists of a T cell.

In some embodiments, the method further comprises administering a drug increasing expression of FLT3 on a cancer cell.

In some aspects, the disclosure is drawn to a method of inhibiting the growth of a cancer cell expressing FLT3 or a tissue comprising the cancer cell. The method comprises, or alternatively consists essentially of, or yet further consists of, contacting the cancer cell or tissue with any of the cells expressing the CARs and optionally the truncated protein maker as disclosed herein. In some embodiments, the contacting is ex vivo or in vitro or in vivo. In some embodiments, the contacting is in vivo and the expressing cells are autologous or allogeneic to a subject being treated. In some embodiments, the contacting is in vivo and the expressing cells are allogenic to a subject being treated. The contacting in vivo can be by administration of the expressing cells to the subject in need of such treatment.

In some aspects, this disclosure provides a method of inhibiting the growth of a cancer cell expressing FLT3 or a tissue in a subject, comprising the cancer cell by administering to the subject, for example an effective amount of, the cells expressing the CAR as described herein and optionally a combined therapy.

In some aspects, the disclosure is drawn to a method of treating a cancer in a subject in need thereof. In some embodiments, the subject comprises a cancer cell expressing FLT3. The method comprises, or alternatively consists essentially of, or yet further consists of, administering to the subject, for example an effective amount of, the cells expressing the CARs and optionally the truncated protein maker as disclosed herein. In some embodiments, the CAR expressing cells are autologous or allogeneic to a subject being treated. In some embodiments, the CAR expressing cells are allogenic to a subject being treated. In some embodiments, the method further comprises administering the subject a combined therapy.

In some aspects, provided herein is a method for one or more of: inhibiting the growth of a cancer or tumor, inhibiting metastasis of a cancer or a tumor, or treating a cancer or a tumor, in a subject in need thereof. The method comprises, or consists essentially of, or yet further consists of administering the cells expressing the CAR and optionally the truncated protein marker to the subject. In some embodiments, the CAR expressing cell is autologous or allogeneic to the subject in need. In some embodiments, the CAR expressing cell is allogenic to the subject in need.

In one aspect, provided is a method for treating a cancer in a subject selected for the treatment. The method comprises, or alternatively consists essentially of, or yet consists of administering, for example an effective amount of, a cell or a cell population as disclosed herein to the subject. In some embodiments, the subject is selected if a cancer cell of the subject expresses FLT3. In some embodiments, the FLT3 expression is determined by contacting a sample, such as a biopsy of a cancer, of the subject with an antibody or an antigen binding domain specifically recognizing and binding the FLT3 in vitro or in vivo, and detecting binding between the sample and the antibody or antigen binding domain. In further embodiments, the antigen binding domain further comprises a detectable marker. In some embodiments, the isolated or engineered cell is autologous to the subject in need. In some embodiments, the isolated or engineered cell is allogenic to the subject in need.

In some aspects, a method as disclosed herein further comprises, or alternatively consists essentially of, or yet further consists of administering to the subject a combined therapy. Appropriate treatment regimens will be determined by the treating physician or veterinarian.

In some embodiments, the FLT3 CAR cells may be administered before or after any one of these non-limiting exemplary combined therapies, e.g., before hematopoietic stem cell transplantation (HSCT) or after radiation therapy or chemotherapy. In embodiments where the FLT3 CAR cells are used before hematopoietic stem cell transplantation, the FLT3 CAR cells may be used to achieve remission prior to the delivery of hematopoietic stem cells; in general, hematopoietic stem cell transplantation is more successful after remission. Further non-limiting examples include other relevant cell types, such as unmodified immune cells, modified immune cells comprising vectors expressing one or more immunoregulatory molecules, or CAR cells specific to a different antigen than those disclosed herein. As with the CAR cells of the present disclosure, in some embodiments, these cells may be autologous or allogeneic.

In some embodiments, the combined therapy is selected from one or both of a cytoreductive therapy or a therapy that upregulates the expression of FLT3 in a cancer cell. In some embodiments, the cytoreductive therapy comprises, or alternatively consists essentially of, or yet further consists of one or more of a chemotherapy, a cryotherapy, a hyperthermia, a targeted therapy, an immunotherapy, or a radiation therapy. In further embodiments, a combined therapy comprises, or alternatively consists essentially of, or yet further consists of physically removal of the cancer cells or tissue comprising the cancer cells. In yet further embodiments, the combined therapy may be used prior to, concurrently with, or after the FLT3-specific CAR therapy as disclosed herein. In some embodiments, the therapy the upregulates the FLT3 expression in a cancer cell is used prior to or concurrently with the FLT3-specific CAR therapy as disclosed herein.

In some embodiments, the immunotherapy refers to enhancing the immune response in the subject to a cancer cell, for example, an anti-PD-1 therapy, or an anti-PD-L1 therapy. Non-limiting examples of commercially available antibodies to PD-1 include pembrolizumab (Merck), nivolumab (Bristol-Myers Squibb), pidilizumab (Cure Tech), AMP-224 (GSK), AMP-514 (GSK), PDR001 (Novartis), and cemiplimab (Regeneron and Sanofi). Non-limiting examples of commercially available antibodies to PD-L1 include atezolizumab (Roche Genentech), avelumab (Merck Soreno and Pfizer), durvalumab (AstraZeneca), BMS-936559 (Bristol-Myers Suibb), and CK-301 (Checkpoint Therapeutics).

In some embodiments, a method as disclosed herein is used as a first line therapy. In other embodiments, a method as disclosed herein is used as a second line therapy or a third line therapy.

In certain embodiments, the patient or subject maintains or recovers normal hematopoiesis after receiving, i.e., being administered, the effective amount of the isolated cell. Normal hematopoiesis is a critical endpoint for certain cancers, such as but not limited to cancers affecting the blood or bone marrow, e.g., lymphoma or leukemia, such as but not limited to acute myeloid leukemia or acute lymphoblastic leukemia. Methods of determining “normal hematopoiesis” after treatment are known in the art and include but are not limited to a “pin prick” blood test comparing baseline blood cell counts to post-treatment blood cell counts and/or similar comparisons for circulating CD34+ cells. Further non-limiting exemplary methods include bone marrow biopsy to verify engraftment. Failure to maintain or recover normal hematopoiesis (also known as normal engraftment) is associated with recurrent need for transfusions and/or need for antibiotics and/or high morbidity and mortality, in addition to symptomatic indicators such as but not limited to anemia, paleness, orthostatic hypotension, and bleeding and/or bruising due to a lack of platelet recovery. Normal hematopoiesis and/or engraftment may be defined by a clinically acceptable threshold, such as but not limited to a sustained granulocyte count of >1.0×10⁹/L, a sustained platelet count of >50×10⁹, a sustained hemoglobin level of ˜9 or 10 g/dL, and/or the absence of a need for red blood cell transfusions. In some embodiments, normal hematopoiesis is defined by a lack of significant depletion of Lin-CD34+CD38− CD90+CD45RA− cells. In some embodiments, adequate long-term hematopoiesis or successful long-term hematopoietic engraftment can be correlated with sufficient numbers of Lin-CD34+CD38−CD90+CD45RA− cells in the hematopoietic product being infused into a subject following myeloablative preparation for stem cell transplantation.

The total dose of CAR expressing cells may vary depending on, for example, the above disclosed factors. In some embodiments, the doses may be on the order of between 1 to 10¹⁰ cells, e.g., at least 1, at least 10¹, at least 10², at least 10³, at least 10⁴, at least 10⁵, at least 10⁶, at least 10⁷, at most 10⁸, at most 10⁹, at most 10¹⁰, between 10² and 10¹⁰, between 10³ and 10⁹, between 10⁴ and 10⁸, per patient or per kilogram (kg) body weight of the patient. In some embodiments, the dose may be further limited by an integer coefficient to the order of magnitude, e.g., 1, 2, 3, 4, 5, 6, 7, 8, or 9, resulting a dose range listed according to the following non-limiting example: between 5×10⁴ and 1×10⁸ per patient or per kg body weight of the patient.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. In one embodiment, they are administered directly by direct injection or systemically such as intravenous injection.

Aspects of the disclosure provide an exemplary method for determining if a patient is likely to respond to, or is not likely to respond to, CAR therapy. The method comprises, or alternatively consists essentially of, or further consists of determining the presence or absence of necrosis in a tumor sample isolated from the patient and quantitating the amount of cancer or tumor cells expressing the cancer or tumor antigen. In certain embodiments, the method further comprises, or alternatively consists essentially of, or yet further consists of administering an effective amount of the CAR therapy to the patient that is determined likely to respond to the CAR therapy. The CAR therapy can be autologous or allogenic to the patient and the patient can be subject that suffers from a cancer, animal or human.

Techniques of histological staining for necrosis are well known in the art. For example, hematoxylin and eosin stains, also referred to as “H&E staining,” are a common technique for identifying the presence of necrosis in tissues, especially in tumorigenic or cancerous growth. Cytoplasmic H&E staining demonstrates increased eosinophilia, attributable in part to the loss of cytoplasmic RNA and in part to denatured cytoplasmic proteins. In necrotic tissue stains, the cytoplasm often appears “moth eaten” due to enzyme digestion of cytoplasmic organelles. Myelin figures, calcification, and evidence of phagocytosis into other cells are also hallmarks of necrotic tissues that can be detected by histological staining. Necrotic tissues also have specific hallmarks in nuclear staining often demonstrating karyolysis, pyknosis, and karyorrhexis as a result of cell death. Using microscopy and either manual or automated quantitation of such necrotic hallmarks, relevance of CAR therapy may be determined. Alternate means of detecting tumorigenic or cancerous growth or necrotic tissues in general, including but not limited to biomarker-based or imaging-based diagnostics, are also equally relevant to determining whether a patient will respond to certain types of CAR therapy, and may be used accordingly. As is apparent, the CAR therapy is selected based on the genotype and/or phenotype of the cancer or tumor in the patient sample such that the antigen binding domain will target and treat the specific cancer or tumor.

### Kits

Also provided herein is a kit that comprises, or alternatively consists essentially of, or yet further consists of, one or more of a CAR, a truncated protein marker, a polynucleotide, a cell, a cell population, or a composition as disclosed herein and optionally, instructions for making or using the same.

In one particular aspect, the present disclosure provides kits for performing these methods as well as instructions for carrying out the methods of the present disclosure such as collecting cells or tissues or both; performing the screening, transduction, etc.; analyzing the results, or any combination thereof.

In one aspect, the kit comprises, or alternatively consists essentially of, or yet further consists of, any one or more of: a polypeptide as disclosed herein, a polynucleotide as disclosed herein, a vector as disclosed herein, a vector comprising said nucleic acid, a cell as disclosed herein, such as isolated allogenic cells, preferably T cells or NK cells, a cell population as disclosed herein, a composition as disclosed herein, an isolated complex as disclosed herein, or instructions optionally on the procuring of autologous cells from a patient. Such a kit may also comprise, or alternatively consist essentially of, or yet further comprise media and other reagents appropriate for the transduction, the selection, the activation, or the expansion of CAR.

In one aspect the kit comprises, or alternatively consists essentially of, or yet further consists of, a CAR expressing cell or a population thereof. In some embodiments, the cells of this kit may require activation or expansion or both prior to administration to a subject in need thereof. In further embodiments, the kit may further comprise, or consist essentially of, media and reagents, such as those covered in the disclosure above, to activate or expand or both activate and expand the isolated CAR expressing cell. In some embodiments, the cell is to be used for a CAR therapy. In further embodiments, the kit comprises instructions on the administration of the isolated cell to a patient in need of CAR therapy.

The kits of this disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

Other features and advantages of the invention will be apparent from the following detailed description and figures, and from the claims.

### EXAMPLES

The invention is further described in the following examples, which do not limit the scope of the invention described in the claims.

### Example 1: Vectors for Expression of FLT3 CAR

A variety of vectors and expression systems can be used to transduce T cells to express a FLT3 CAR. One suitable vector is depicted in FIG. 2. In this lentiviral vector, which is based on the pHIV7 vector (Chung et al. Mol Ther. 2014 May; 22(5): 952-963. Epub 2014 Feb. 28., FLT3 CAR expression is driven by the EF-1alpha promoter and the FLT3 CAR is co-expressed with a truncated CD19, which can be used as a marker.

An alternative vector for FLT3 CAR expression is depicted in FIG. 3. Here expression is driven by an MMLV promoter and the FLT3 CAR is co-expressed with a truncated CD19, which can be used as a marker.

### Example 2: Improved FLT3 CAR Expression

PBMC enriched for CD4+ and CD8+ cells were activated using CD3/CD28 beads. The following day the cells were transduced with a vector expressing a FLT3-specific CAR encoded by the improved coding sequence (SEQ ID NO: 1) or an otherwise identical vector expressing the same FLT3-specific CAR, but encoded by a previously used coding sequence. The vectors used the pHIV7 backbone. The FLT3-specific CAR were co-expressed with a CD19t tag, as described herein. Two controls were used: a vector expressing GFP and a vector expressing the CD19t tag. In all cases the T cells were transfected at a MOI of 1. FLT3 CAR-T expression and cytotoxicity were performed on Day 5.

As can be seen in FIGS. 4A-4D, the highest CAR expression was seen when the T cells were transduced with the improved FLT3-specific CAR coding sequence.

### Example 3: Improved Killing of FLT3+ Tumor Cells

Cytotoxicity of T cells transduced with the various constructs was evaluated at 24 h and 72 h using MOLM13 cells as FLT3+ tumor target and U937 cells as a negative control. The E:T ratios were 1:5 and 1:25. Results are shown as averages of data from the 3 healthy donors as the % tumor lysis±SEM. As can be seen in FIGS. 5A-5B, T cells transduced with the improved FLT3-specific CAR coding sequence were more effective than T cells transduced with the earlier FLT3-specific CAR coding sequence.

### Example 4: Improved Cytokine Expression

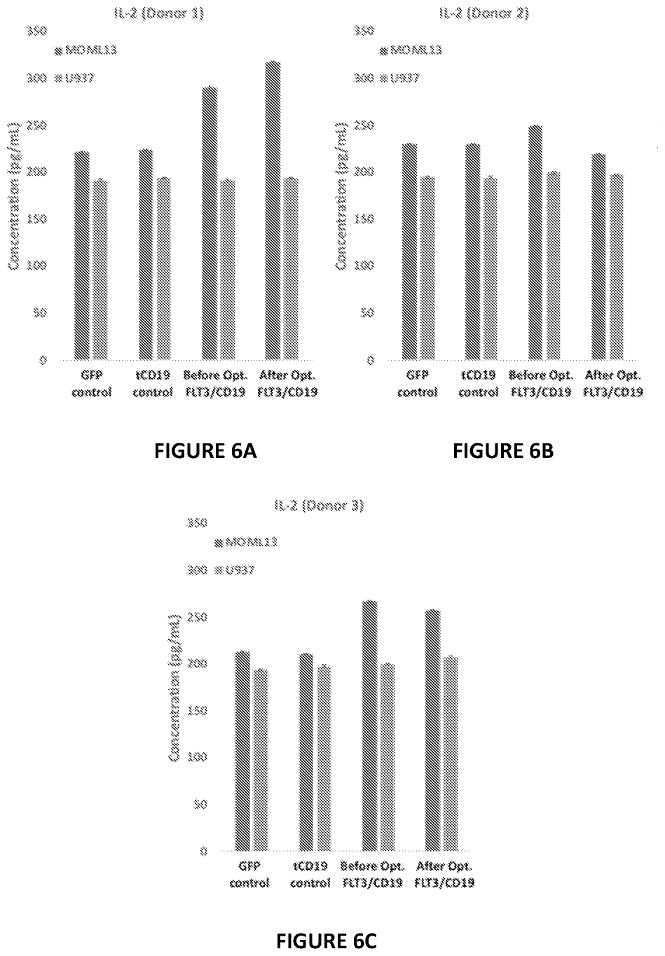

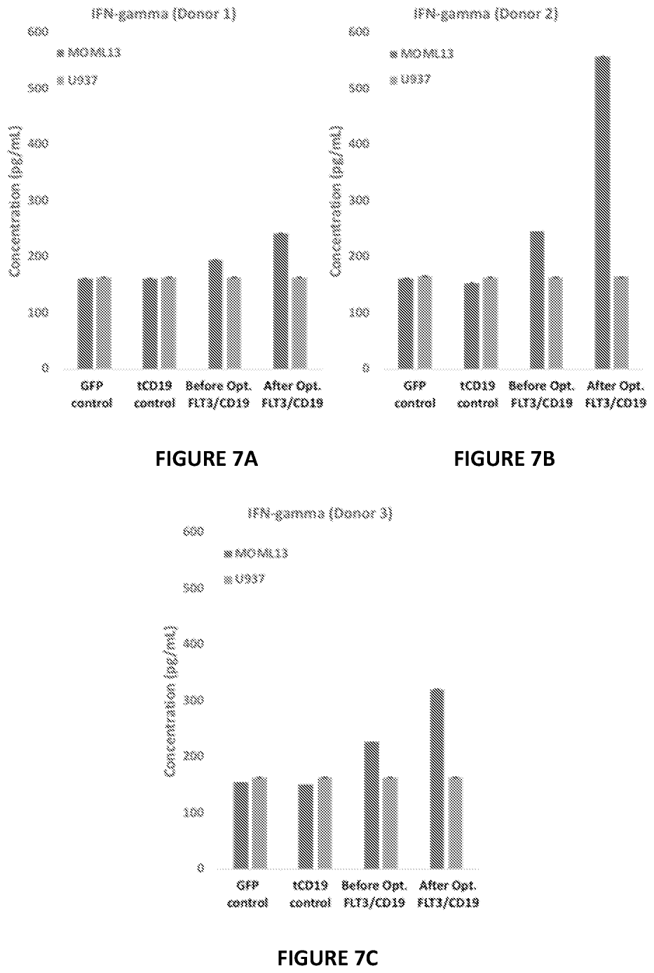

Tumor cells were obtained from 3 patients and were co-cultured with T cells transduced with the various constructs. The expression of IL-2 (FIGS. 6A-6C) and IFN-γ (FIGS. 7A-7C) in culture supernatants was measured. As can be seen in FIGS. 4A-4D and FIGS. 5A-5B, despite some interpatient variability, T cells transduced with the improved FLT3-specific CAR coding sequence were superior to T cells transduced with the earlier FLT3-specific CAR coding sequence.

### Example 5: FLT3 CAR Activity in a Mouse Model of Aggressive Human FLT3(+) AML (MOLM-13)

1×10⁵ MOLM-13 cells expressing luciferase were injected into 11-week-old male NSG mice on day 0. Viably frozen and thawed FLT3 CAR NK cells (20×10⁶) and viably frozen FLT3 CAR T cells (5×10⁶) cells were injected IV on day 0, one hour after the MOLM-13 injection. Mice receiving FLT3 CAR NK cells were divided into two groups: one receiving a single IV weekly injection of FLT3 CAR NK cells, the other group receiving 3 doses of FLT3 CAR-NK cells over 3 consecutive days weekly (Day 0, 1, 2, and 7, 8, 9, etc), and the last group receiving a single dose of FLT3 CAR T cells (with relapse appearing at Day 21).

### Embodiments

Embodiment 1. A nucleic molecule comprising the nucleotide sequence of SEQ ID NO:1.

Embodiment 2. A viral vector or an expression vector comprising the nucleic acid molecule of Embodiment 1.

Embodiment 3. The nucleic acid molecule of Embodiment 1, further comprising a T2A skip sequence and a sequence encoding a truncated EGFR or a truncated CD19.

Embodiment 4. The nucleic acid molecule of Embodiment 1, further comprising a T2A skip sequence and a sequence encoding a truncated EGFR.

Embodiment 5. The nucleic acid molecule of Embodiment 1, further comprising a T2A skip sequence and a sequence encoding a truncated CD19.

Embodiment 6. The nucleic acid molecule of Embodiment 1, further comprising a T2A skip sequence and a sequence encoding a truncated EGFR.

Embodiment 7. The nucleic acid molecule of Embodiment 1, further comprising a T2A skip sequence and a sequence encoding a truncated CD19.

Embodiment 8. The vector of Embodiment 2, wherein the vector is a lentiviral vector.

Embodiment 9. The vector of Embodiment 2, wherein the vector is a retroviral vector.

Embodiment 10. The vector of Embodiment 2, wherein expression of nucleotide sequence of SEQ ID NO: 1 is under the control of an EF-1alpha promoter or a MMLV promoter.

Embodiment 11. A population of human T cells transduced by a vector comprising the nucleic acid molecule of Embodiment 1.

Embodiment 12. The population of human T cells of Embodiment 11, wherein the population of human T cells comprise central memory T cells, NK cells, naive memory T cells, pan T cells, or PBMC substantially depleted for CD25+ cells and CD14+ cells.

Embodiment 13. A method of treating a patient suffering from acute myeloid leukemia, comprising administering a population of autologous or allogeneic human T cells transduced by a vector comprising the nucleic acid molecule of Embodiment 1.

Embodiment 14. The method of Embodiment 13, wherein the patient harbors tumor cell having an activating mutation in Fms-like tyrosine kinase 3 (FLT3).

Embodiment 15. The method of Embodiment 13, wherein the patient harbors tumor cell having an FLT3-ITD mutation.

Embodiment 16. The method of Embodiment 13, wherein Embodiment chimeric antigen receptor is administered locally or systemically.

Embodiment 17. The method of Embodiment 13, wherein the chimeric antigen receptor is administered by single or repeat dosing.

Embodiment 18. A method of preparing CAR T cells comprising: providing a population of autologous or allogeneic human T cells and transducing the T cells by a vector comprising the nucleic acid molecule of Embodiment 1.

Sequence Listing
SEQ ID NO: 1:
optimized CAR coding sequence,
ATGGGGTGGTCAAGCATTATTCTGTTTCTGGTC
GCTACCGCTACAGGCGTCCATCAGGTCCAGCTG
CAGCAGCCCGGAGCCGAACTGGTGAAGCCCGGC
GCCTCCCTGAAGCTGTCTTGCAAGAGCAGCGGC
TACACATTCACCTCCTATTGGATGCACTGGGTG
CGGCAGCGGCCCGGCCACGGCCTGGAGTGGATC
GGCGAGATCGACCCCTCTGATAGCTACAAGGAC
TATAACCAGAAGTTTAAGGATAAGGCCACACTG
ACCGTGGACCGGTCTAGCAATACAGCCTACATG
CACCTGTCCTCTCTGACCTCCGACGATTCTGCC
GTGTACTATTGCGCCAGGGCCATCACCACAACC
CCTTTCGATTTTTGGGGCCAGGGCACAACCCTG
ACCGTGAGCAGCGGAGGAGGAGGCAGCGGAGGA
GGAGGCTCCGGCGGCGGCGGCTCTGACATCGTG
CTGACCCAGTCCCCAGCCACACTGAGCGTGACC
CCTGGCGACTCCGTGTCTCTGAGCTGTCGGGCC
TCCCAGTCTATCAGCAACAATCTGCACTGGTAT
CAGCAGAAGAGCCACGAGTCCCCTAGGCTGCTG
ATCAAGTATGCCTCCCAATCTATCAGCGGCATC
CCAAGCCGCTTCTCCGGCTCTGGCAGCGGCACA
GACTTCACCCTGTCTATCAACAGCGTGGAGACA
GAGGACTTCGGCGTGTATTTTTGTCAGCAGTCT
AATACATGGCCATATACATTCGGAGGAGGAACT
AAACTGGAAATCAAACGACTCGAGCCCAAATCT
TGTGACAAAACTCACACATGCCCACCGTGCCCG
GATCCCAAAggtaccTTTTGGGTGCTGGTGGTG
GTTGGTGGAGTCCTGGCTTGCTATAGCTTGCTA
GTAACAGTGGCCTTTATTATTTTCTGGGTGAGG
AGTAAGAGGAGCAGGCTCCTGCACAGTGACTAC
ATGAACATGACTCCCCGCCGCCCCGGGCCCACC
CGCAAGCATTACCAGCCCTATGCCCCACCACGC
GACTTCGCAGCCTATCGCTCCAGAGTGAAGTTC
AGCAGGAGCGCAGACGCCCCCGCGTACCAGCAG
GGCCAGAACCAGCTCTATAACGAGCTCAATCTA
GGACGAAGAGAGGAGTACGATGTTTTGGACAAG
AGACGTGGCCGGGACCCTGAGATGGGGGGAAAG
CCGAGAAGGAAGAACCCTCAGGAAGGCCTGTAC
AATGAACTGCAGAAAGATAAGATGGCGGAGGCC
TACAGTGAGATTGGGATGAAAGGCGAGCGCCGG
AGGGGCAAGGGGCACGATGGCCTTTACCAGGGT
CTCAGTACAGCCACCAAGGACACCTACGACGCC
CTTCACATGCAGGCCCTGCCCCCTCGC
SEQ ID NO: 2: an FLT3 CAR,
MGWSSIILFLVATATGVHQVQLQQPGAELVKPG
ASLKLSCKSSGYTFTSYWMHWVRQRPGHGLEWI
GEIDPSDSYKDYNQKFKDKATLTVDRSSNTAYM
HLSSLTSDDSAVYYCARAITTTPFDFWGQGTTL
TVSSGGGGSGGGGSGGGGSDIVLTQSPATLSVT
PGDSVSLSCRASQSISNNLHWYQQKSHESPRLL
IKYASQSISGIPSRFSGSGSGTDFTLSINSVET
EDFGVYFCQQSNTWPYTFGGGTKLEIKRLEPKS
CDKTHTCPPCPDPKGTFWVLVVVGGVLACYSLL
VTVAFIIFWVRSKRSRLLHSDYMNMTPRRPGPT
RKHYQPYAPPRDFAAYRSRVKFSRSADAPAYQQ
GQNQLYNELNLGRREEYDVLDKRRGRDPEMGGK
PRRKNPQEGLYNELQKDKMAEAYSEIGMKGERR
RGKGHDGLYQGLSTATKDTYDALHMQALPPR
SEQ ID NO: 3: a signal sequence,
MGWSSIILFLVATATGVH
SEQ ID NO: 4: an FLT3 scFv, wherein amino acid
(aa) 1 to aa 118 of SEQ ID NO: 4 is a heavy
chain variable region, aa 134 to aa 241 of
SEQ ID NO: 4 is a light chain variable region,
and aa 119 to aa 133 of SEQ ID NO: 4 is a
peptide linker.
QVQLQQPGAELVKPGASLKLSCKSSGYTFTSYW
MHWVRQRPGHGLEWIGEIDPSDSYKDYNQKFKD
KATLTVDRSSNTAYMHLSSLTSDDSAVYYCARA
ITTTPFDFWGQGTTLTVSSGGGGSGGGGSGGGG
SDIVLTQSPATLSVTPGDSVSLSCRASQSISNN
LHWYQQKSHESPRLLIKYASQSISGIPSRFSGS
GSGTDFTLSINSVETEDFGVYFCQQSNTWPYTF
GGGTKLEIKR
SEQ ID NO: 5: an IgG1 hinge domain,
LEPKSCDKTHTCPPCPDPKGT
SEQ ID NO: 6: a CD28 transmembrane domain,
FWVLVVVGGVLACYSLLVTVAFIIFWV
SEQ ID NO: 7: a CD28 costimulatory domain,
RSKRSRLLHSDYMNMTPRRPGPTRKHYQPYAPPRDFAAYRS
SEQ ID NO: 8:
a CD3zeta intracellular signaling domain,
RVKFSRSADAPAYQQGQNQLYNELNLGRREEYD
VLDKRRGRDPEMGGKPRRKNPQEGLYNELQKDK
MAEAYSEIGMKGERRRGKGHDGLYQGLSTATKD
TYDALHMQALPPR
SEQ ID NO: 9: a ribosomal skip sequence,
LEGGGEGRGSLLTCGDVEENPGPR
SEQ ID NO: 10: rEGFR,
LVTSLLLCELPHPAFLLIPRKVCNGIGIGEFKD
SLSINATNIKHFKNCTSISGDLHILPVAFRGDS
FTHTPPLDPQELDILKTVKEITGFLLIQAWPEN
RTDLHAFENLEIIRGRTKQHGQFSLAVVSLNIT
SLGLRSLKEISDGDVIISGNKNLCYANTINWKK
LFGTSGQKTKIISNRGENSCKATGQVCHALCSP
EGCWGPEPRDCVSCRNVSRGRECVDKCNLLEGE
PREFVENSECIQCHPECLPQAMNITCTGRGPDN
CIQCAHYIDGPHCVKTCPAGVMGENNTLVWKYA
DAGHVCHLCHPNCTYGCTGPGLEGCPTNGPKIP
SIATGMVGALLLLLVVALGIGLFM
SEQ ID NO: 11: tCD19,
MPPPRLLFFLLFLTPMEVRPEEPLVVKVEEGDN
AVLQCLKGTSDGPTQQLTWSRESPLKPFLKLSL
GLPGLGIHMRPLAIWLFIFNVSQQMGGFYLCQP
GPPSEKAWQPGWTVNVEGSGELFRWNVSDLGGL
GCGLKNRSSEGPSSPSGKLMSPKLYVWAKDRPE
IWEGEPPCLPPRDSLNQSLSQDLTMAPGSTLWL
SCGVPPDSVSRGPLSWTH
SEQ ID NO: 12: consensus sequence, wherein
the small letter indicates any
nucleotide or a nucleotide located in the
corresponding position in SEQ ID NO: 1,
ATGGGgTGGtcaagcATtATtCTgTTtcTGGTc
GCtACcGCTACAGGcGTCCAtCAGGTCCAgCTG
CAGCAGCCcGGaGCcGAaCTgGTGAAGCCcGGc
GCcTCccTGAAGCTGTCtTGCAAGagcagCGGc
TACACaTTCACCtcCTAtTGGATGCACTGGGTG
cGGCAGcGGCCcGGcCAcGGCCTgGAGTGGATC
GGcGAGATcGAcCCcTCTGAtAGcTAcAAgGAC
TAtAAcCAGAAGTTtAAGGAtAAGGCCACAcTG
ACcGTGGACcGgTCtagCAAtACAGCCTACATG
CACCTgtcCtctCTGACcTCcGAcGAtTCTGCc
GTgTAcTATTGcGCcAGgGCcATcACcACaACC
CCtTTcGAtTTtTGGGGCCAgGGCACaACcCTg
ACcGTgagCagcGGaGGaGGaGGcagcGGaGGa
GGaGGCTCCGGCGGcGGCGGcTCTGAcATcGTG
CTgACcCAGTCcCCAGCCACaCTGagcGTGACc
CCtGGcGActcCGTgtcTCTgagCTGtcGGGCC
tcCCAGtcTATcAGCAACAAtCTgCACTGGTAT
CAgCAgAAgagcCAcGAGTCcCCtAGGCTgCTg
ATCAAGTATGCcTCCCAaTCtATCagcGGcATC
CCaagCcGcTTCtccGGCtcTGGcagcGGcACA
GAcTTCACcCTgtcTATCAACAGcGTGGAGACa
GAgGAcTTcGGcGTGTATTTtTGTCAgCAGtcT
AAtACaTGGCCaTAtACaTTCGGAGGaGGaACt
AAaCTGGAAATcAAACGactcgagcccaaatct
tgtgacaaaactcacacatgcccaccgtgcccg
gatcccaaaggtaccttttgggtgctggtggtg
gttggtggagtcctggcttgctatagcttgcta
gtaacagtggcctttattattttctgggtgagg
agtaagaggagcaggctcctgcacagtgactac
atgaacatgactccccgccgccccgggcccacc
cgcaagcattaccagccctatgccccaccacgc
gacttcgcagcctatcgctccagagtgaagttc
agcaggagcgcagacgcccccgcgtaccagcag
ggccagaaccagctctataacgagctcaatcta
ggacgaagagaggagtacgatgttttggacaag
agacgtggccgggaccctgagatggggggaaag
ccgagaaggaagaaccctcaggaaggcctgtac
aatgaactgcagaaagataagatggcggaggcc
tacagtgagattgggatgaaaggcgagcgccgg
aggggcaaggggcacgatggcctttaccagggt
ctcagtacagccaccaaggacacctacgacgcc
cttcacatgcaggccctgccccctcgc
SEQ ID NO: 13: tCD19,
MPPPRLLFFLLFLTPMEVRPEEPLVVKVEEGDN
AVLQCLKGTSDGPTQQLTWSRESPLKPFLKLSL
GLPGLGIHMRPLAIWLFIFNVSQQMGGFYLCQP
GPPSEKAWQPGWTVNVEGSGELFRWNVSDLGGL
GCGLKNRSSEGPSSPSGKLMSPKLYVWAKDRPE
IWEGEPPCVPPRDSLNQSLSQDLTMAPGSTLWL
SCGVPPDSVSRGPLSWTHVHPKGPKSLLSLELK
DDRPARDMWVMETGLLLPRATAQDAGKYYCHRG
NLTMSFHLEITARPVLWHWLLRTGGWKVSAVTL
AYLIFCLCSLVGILHLQRALVLRRKR
SEQ ID NO: 14:
an scFv coding sequence prior to
optimization
ATGGGATGGAGCTCTATCATCCTCTTCTTGGTA
GCAACAGCTACAGGTGTCCACCAGGTCCAACTG
CAGCAGCCTGGGGCTGAGCTTGTGAAGCCTGGG
GCTTCATTGAAGCTGTCCTGCAAGTCTTCCGGG
TACACCTTCACCAGCTACTGGATGCACTGGGTG
AGGCAGAGGCCTGGACATGGCCTTGAGTGGATC
GGAGAGATTGATCCTTCTGACAGTTATAAAGAC
TACAATCAGAAGTTCAAGGACAAGGCCACATTG
ACTGTGGACAGATCCTCCAACACAGCCTACATG
CACCTCAGCAGCCTGACATCTGATGACTCTGCG
GTCTATTATTGTGCAAGAGCGATTACGACGACC
CCCTTTGACTTCTGGGGCCAAGGCACCACTCTC
ACAGTCTCCTCAGGCGGTGGCGGTTCTGGTGGC
GGTGGCTCCGGCGGTGGCGGTTCTGATATTGTG
CTAACTCAGTCTCCAGCCACCCTGTCTGTGACT
CCAGGAGATAGCGTCAGTCTTTCCTGCAGGGCC
AGCCAGAGTATTAGCAACAACCTACACTGGTAT
CAACAAAAATCACATGAGTCTCCAAGGCTTCTC
ATCAAGTATGCTTCCCAGTCCATCTCTGGGATC
CCCTCCAGGTTCAGTGGCAGTGGATCAGGGACA
GATTTCACTCTCAGTATCAACAGTGTGGAGACT
GAAGATTTTGGAGTGTATTTCTGTCAACAGAGT
AACACCTGGCCGTACACGTTCGGAGGGGGGACC
AAGCTGGAAATAAAACGG
SEQ ID NO: 15: CDRL1, RASQSISNNLH
SEQ ID NO: 16: CDRL2, YASQSIS
SEQ ID NO: 17: CDRL3, QQSNTWPYT
SEQ ID NO: 18: CDRH1, SYWMH
SEQ ID NO: 19: CDRH2, EIDPSDSYKDYNQKFKD
SEQ ID NO: 20: CDRH3, AITTTPFDF
SEQ ID NO: 21:
an FLT3 heavy chain variable region,
QVQLQQPGAELVKPGASLKLSCKSSGYTFTSY
WMHWVRQRPGHGLEWIGEIDPSDSYKDYNQKF
KDKATLTVDRSSNTAYMHLSSLTSDDSAVYYC
ARAITTTPFDFWGQGTTLTVSS
SEQ ID NO: 22:
an FLT3 light chain variable region,
DIVLTQSPATLSVTPGDSVSLSCRASQSISNNLH
WYQQKSHESPRLLIKYASQSISGIPSRFSGSGSG
TDFTLSINSVETEDFGVYFCQQSNTWPYTFGGGT
KLEIKR
SEQ ID NO: 23: EF1 alpha promoter
AAGGATCTGCGATCGCTCCGGTGCCCGTCAGTG
GGCAGAGCGCACATCGCCCACAGTCCCCGAGAA
GTTGGGGGGAGGGGTCGGCAATTGAACGGGTGC
CTAGAGAAGGTGGCGCGGGGTAAACTGGGAAAG
TGATGTCGTGTACTGGCTCCGCCTTTTTCCCGA
GGGTGGGGGAGAACCGTATATAAGTGCAGTAGT
CGCCGTGAACGTTCTTTTTCGCAACGGGTTTGC
CGCCAGAACACAGCTGAAGCTTCGAGGGGCTCG
CATCTCTCCTTCACGCGCCCGCCGCCCTACCTG
AGGCCGCCATCCACGCCGGTTGAGTCGCGTTCT
GCCGCCTCCCGCCTGTGGTGCCTCCTGAACTGC
GTCCGCCGTCTAGGTAAGTTTAAAGCTCAGGTC
GAGACCGGGCCTTTGTCCGGCGCTCCCTTGGAG
CCTACCTAGACTCAGCCGGCTCTCCACGCTTTG
CCTGACCCTGCTTGCTCAACTCTACGTCTTTGT
TTCGTTTTCTGTTCTGCGCCGTTACAGATCCAA
GCTGTGACCGGCGCCTAC
SEQ ID NO: 24: MMLV promoter,
TTAATTAAGT AACGCCATTT TGCAAGGCAT
GGAAAAATAC ATAACTGAGA ATAGAGAAGT
TCAGATCAAG GTCAGGAACA GATGGAACAG
CTGAATATGG GCCAAACAGG ATATCTGTGG
TAAGCAGTTC CTGCCCCGGC TCAGGGCCAA
GAACAGATGG AACAGCTGAA TATGGGCCAA
ACAGGATATC TGTGGTAAGC AGTTCCTGCC
CCGGCTCAGG GCCAAGAACA GATGGTCCCC
AGATGCGGTC CAGCCCTCAG CAGTTTCTAG
AGAACCATCA GATGTTTCCA GGGTGCCCCA
AGGACCTGAA ATGACCCTGT GCCTTATTTG
AACTAACCAA TCAGTTCGCT TCTCGCTTCT
GTTCGCGCGC TTCTGCTCCC CGAGCTCAAT
AAAAGAGCCC ACAACCCCTC ACTCGGGGCG
CCAGTCCTCC GATTGACTGA GTCGCCCGCT
TAAG
SEQ ID NO: 25: FLT3 isoform 1,
MPALARDGGQLPLLVVFSAMIFGTITNQDLPVI
KCVLINHKNNDSSVGKSSSYPMVSESPEDLGCA
LRPQSSGTVYEAAAVEVDVSASITLQVLVDAPG
NISCLWVFKHSSLNCQPHFDLQNRGVVSMVILK
MTETQAGEYLLFIQSEATNYTILFTVSIRNTLL
YTLRRPYFRKMENQDALVCISESVPEPIVEWVL
CDSQGESCKEESPAVVKKEEKVLHELFGTDIRC
CARNELGRECTRLFTIDLNQTPQTTLPQLFLKV
GEPLWIRCKAVHVNHGFGLTWELENKALEEGNY
FEMSTYSTNRTMIRILFAFVSSVARNDTGYYTC
SSSKHPSQSALVTIVEKGFINATNSSEDYEIDQ
YEEFCFSVRFKAYPQIRCTWTFSRKSFPCEQKG
LDNGYSISKFCNHKHQPGEYIFHAENDDAQFTK
MFTLNIRRKPQVLAEASASQASCFSDGYPLPSW
TWKKCSDKSPNCTEEITEGVWNRKANRKVFGQW
VSSSTLNMSEAIKGFLVKCCAYNSLGTSCETIL
LNSPGPFPFIQDNISFYATIGVCLLFIVVLTLL
ICHKYKKQFRYESQLQMVQVTGSSDNEYFYVDF
REYEYDLKWEFPRENLEFGKVLGSGAFGKVMNA
TAYGISKTGVSIQVAVKMLKEKADSSEREALMS
ELKMMTQLGSHENIVNLLGACTLSGPIYLIFEY
CCYGDLLNYLRSKREKFHRTWTEIFKEHNFSFY
PTFQSHPNSSMPGSREVQIHPDSDQISGLHGNS
FHSEDEIEYENQKRLEEEEDLNVLTFEDLLCFA
YQVAKGMEFLEFKSCVHRDLAARNVLVTHGKVV
KICDFGLARDIMSDSNYVVRGNARLPVKWMAPE
SLFEGIYTIKSDVWSYGILLWEIFSLGVNPYPG
IPVDANFYKLIQNGFKMDQPFYATEEIYIIMQS
CWAFDSRKRPSFPNLTSFLGCQLADAEEAMYQN
VDGRVSECPHTYQNRRPFSREMDLGLLSPQAQV
EDS
SEQ ID NO: 26: FLT3 isoform 2,
MPALARDGGQLPLLVVFSAMIFGTITNQDLPVI
KCVLINHKNNDSSVGKSSSYPMVSESPEDLGCA
LRPQSSGTVYEAAAVEVDVSASITLQVLVDAPG
NISCLWVFKHSSLNCQPHFDLQNRGVVSMVILK
MTETQAGEYLLFIQSEATNYTILFTVSIRNTLL
YTLRRPYFRKMENQDALVCISESVPEPIVEWVL
CDSQGESCKEESPAVVKKEEKVLHELFGTDIRC
CARNELGRECTRLFTIDLNQTPQTTLPQLFLKV
GEPLWIRCKAVHVNHGFGLTWELENKALEEGNY
FEMSTYSTNRTMIRILFAFVSSVARNDTGYYTC
SSSKHPSQSALVTIVEKGFINATNSSEDYEIDQ
YEEFCFSVRFKAYPQIRCTWTFSRKSFPCEQKG
LDNGYSISKFCNHKHQPGEYIFHAENDDAQFTK
MFTLNIRRKPQVLAEASASQASCFSDGYPLPSW
TWKKCSDKSPNCTEEITEGVWNRKANRKVFGQW
VSSSTLNMSEAIKGFLVKCCAYNSLGTSCETIL
LNSPGPFPFIQDNISFYATIGVCLLFIVVLTLL
ICHKYKKQFRYESQLQMVQVTGSSDNEYFYVDF
REYEYDLKWEFPRENLEFGKVLGSGAFGKVMNA
TAYGISKTGVSIQVAVKMLKEKADSSEREALMS
ELKMMTQLGSHENIVNLLGACTLSGPIYLIFEY
CCYGDLLNYLRSKREKFHRTWTEIFKEHNFSFY
PTFQSHPNSSMPGSREVQIHPDSDQISGLHGNS
FHSEDEIEYENQKRLEEEEDLNVLTFEDLLCFA
YQVAKGMEFLEFKSARLPVKWMAPESLFEGIYT
IKSDVWSYGILLWEIFSLGVNPYPGIPVDANFY
KLIQNGFKMDQPFYATEEIYIIMQSCWAFDSRK
RPSFPNLTSFLGCQLADAEEAMYQNVDGRVSEC
PHTYQNRRPFSREMDLGLLSPQAQVEDS
SEQ ID NO: 27: a peptide linker, GGGGS
SEQ ID NO: 28: a tCD19 coding sequence,
ATGCCACCTCCTCGCCTCCTCTTCTTCCTCCTC
TTCCTCACCCCCATGGAAGTCAGGCCCGAGGAA
CCTCTAGTGGTGAAGGTGGAAGAGGGAGATAAC
GCTGTGCTGCAGTGCCTCAAGGGGACCTCAGAT
GGCCCCACTCAGCAGCTGACCTGGTCTCGGGAG
TCCCCGCTTAAACCCTTCTTAAAACTCAGCCTG
GGGCTGCCAGGCCTGGGAATCCACATGAGGCCC
CTGGCCATCTGGCTTTTCATCTTCAACGTCTCT
CAACAGATGGGGGGCTTCTACCTGTGCCAGCCG
GGGCCCCCCTCTGAGAAGGCCTGGCAGCCTGGC
TGGACAGTCAATGTGGAGGGCAGCGGGGAGCTG
TTCCGGTGGAATGTTTCGGACCTAGGTGGCCTG
GGCTGTGGCCTGAAGAACAGGTCCTCAGAGGGC
CCCAGCTCCCCTTCCGGGAAGCTCATGAGCCCC
AAGCTGTATGTGTGGGCCAAAGACCGCCCTGAG
ATCTGGGAGGGAGAGCCTCCGTGTcTCCCACCG
AGGGACAGCCTGAACCAGAGCCTCAGCCAGGAC
CTCACCATGGCCCCTGGCTCCACACTCTGGCTG
TCCTGTGGGGTACCCCCTGACTCTGTGTCCAGG
GGCCCCCTCTCCTGGACCCAT
SEQ ID NO: 29: a 2A peptide consensus motif,
D-V/I-E-X-N-P-G-P,
wherein X refers to
any amino acid
SEQ ID NO: 30: a CMV promoter,
ATCGATTGGCTCATGTCCAACATTACCGCCATG
TTGACATTGATTATTGACTAGTTATTAATAGTA
ATCAATTACGGGGTCATTAGTTCATAGCCCATA
TATGGAGTTCCGCGTTACATAACTTACGGTAAA
TGGCCCGCCTGGCTGACCGCCCAACGACCCCCG
CCCATTGACGTCAATAATGACGTATGTTCCCAT
AGTAACGCCAATAGGGACTTTCCATTGACGTCA
ATGGGTGGAGTATTTACGGTAAACTGCCCACTT
GGCAGTACATCAAGTGTATCATATGCCAAGTAC
GCCCCCTATTGACGTCAATGACGGTAAATGGCC
CGCCTGGCATTATGCCCAGTACATGACCTTATG
GGACTTTCCTACTTGGCAGTACATCTACGTATT
AGTCATCGCTATTACCATGGTGATGCGGTTTTG
GCAGTACATCAATGGGCGTGGATAGCGGTTTGA
CTCACGGGGATTTCCAAGTCTCCACCCCATTGA
CGTCAATGGGAGTTTGTTTTGGCACCAAAATCA
ACGGGACTTTCCAAAATGTCGTAACAACTCCGC
CCCATTGACGCAAATGGGCGGTAGGCGTGTACG
GAATT

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A nucleic acid molecule encoding an anti-FMS-like tyrosine kinase 3 (FLT3) antigen binding fragment comprising a polynucleotide having at least 85% identity to the polynucleotide of nucleotide (nt) 55 to nt 777 of SEQ ID NO:1 or the polynucleotide of nt 55 to nt 777 of SEQ ID NO: 12.

2. (canceled)

3. The nucleic acid molecule of claim 1 encoding an anti-FMS-like tyrosine kinase 3 (FLT3) chimeric antigen receptor (CAR), wherein the CAR comprises the polynucleotide of SEQ ID NO: 1 or an equivalent thereof which is at least 85% identical to SEQ ID NO: 1 or the polynucleotide of SEQ ID NO: 12.

4. The nucleic acid molecule of claim 1, wherein the nucleic acid molecule comprises:
a polynucleotide encoding a light chain Complementarity-determining region 1 (CDRL1) as set forth in RASQSISNNLH (SEQ ID NO: 15),
a polynucleotide encoding a light chain Complementarity-determining region 2 (CDRL2) as set forth in YASQSIS (SEQ ID NO: 16),
a polynucleotide encoding a light chain Complementarity-determining region 3 (CDRL3) as set forth in QQSNTWPYT (SEQ ID NO: 17),
a polynucleotide encoding a heavy chain Complementarity-determining region 1 (CDRH1) as set forth in SYWMH (SEQ ID NO: 18),
a polynucleotide encoding a heavy chain Complementarity-determining region 1 (CDRH2) as set forth in EIDPSDSYKDYNQKFKD (SEQ ID NO: 19), and
a polynucleotide encoding a heavy chain Complementarity-determining region 1 (CDRH3) as set forth in AITTTPFDF (SEQ ID NO: 20).

5. The nucleic acid molecule of claim 1, wherein the nucleic acid molecule comprises a polynucleotide encoding an antibody heavy chain variable region of QVQLQQPGAELVKPGASLKLSCKSSGYTFTSYWMHWVRQRPGHGLEWIGEIDPSDSYK DYNQKFKDKATLTVDRSSNTAYMHLSSLTSDDSAVYYCARAITTTPFDFWGQGTTLTV SS (SEQ ID NO: 21) and an antibody light chain variable region of
(SEQ ID NO: 22)
DIVLTQSPATLSVTPGDSVSLSCRASQSISNNLH
WYQQKSHESPRLLIKYASQSISGIPSRFSGSGSG
TDFTLSINSVETEDFGVYFCQQSNTWPYTFGGG
TKLEIKR.

6-10. (canceled)

11. The nucleic acid molecule of claim 1, wherein the nucleic acid molecule encodes a polypeptide selected from the polypeptide of aa 1 to aa 259 of SEQ ID NO: 2, the polypeptide of aa 19 to aa 259 of SEQ ID NO: 2, or the polypeptide of SEQ ID NO: 2.

12. (canceled)

13. The nucleic acid molecule of claim 1, further comprising a polynucleotide encoding a truncated CD19, or a truncated EGFR, wherein the truncated EGFR comprises the polypeptide of SEQ ID NO: 10, and wherein the truncated CD19 comprises the polypeptide of SEQ ID NO: 13 or 11.

14. (canceled)

15. The nucleic acid molecule of claim 1, further comprising a polynucleotide of SEQ ID NO: 28 or an equivalent thereof encoding a truncated CD19.

16. The nucleic acid molecule of claim 13, further comprising a polynucleotide encoding a ribosomal skip sequence located between the polynucleotide encoding the CAR and the polynucleotide encoding the truncated CD19 or the truncated EGFR, wherein the ribosomal skip sequence is a T2A skip sequence.

17. (canceled)

18. (canceled)

19. (canceled)

20. A vector comprising the nucleic acid molecule of claim 1 or a complementary nucleic acid molecule thereof, wherein the vector is a viral vector or an expression vector, optionally wherein the vector is a lentiviral vector or wherein the vector is a retroviral vector.

21. The vector of claim 20, wherein expression of the nucleotide sequence of SEQ ID NO: 1 is under the control of a promoter, wherein the promoter is selected from an EF-1alpha promoter, a CMV promoter, or a MMLV promoter.

22. A population of human T or NK cells comprising the nucleic acid molecule of claim 1.

23. The population of human T or NK cells of claim 22, wherein the population of human T or NK cells comprise central memory T cells, NK cells, naive memory T cells, pan T cells, or PBMC substantially depleted for CD25+ cells and CD14+ cells; wherein the population comprises at least 90% of cells of the same at least one cell subtype.

24. (canceled)

25. An isolated cell comprising the nucleic acid molecule of claim 1, wherein the cell is selected from the group of: an immune cell, an NK cell, a T cell, an NKT cell, a macrophage, a gamma-delta T cell, a stem cell, a progenitor cell or a precursor cell.

26. (canceled)

27. A population of cells comprising the isolated cell of claim 25.

28. A composition comprising the population of claim 22, and a carrier, and optionally a stabilizer, preservative or cryopreservative.

29. A method of treating a human patient suffering from acute myeloid leukemia, comprising administering a population of autologous or allogeneic human T or NK cells comprising the nucleic acid molecule of claim 1.

30. A method of treating a patient in need thereof, comprising administering a cell population of claim 22, wherein the patient having a disease selected from the group of brain cancer, renal cancer, breast cancer, adenocarcinoma, neurological cancer, lung cancer, colorectal cancer, glioblastoma (GBM), melanoma, carcinoid, ovarian cancer, cervical cancer, pancreatic cancer, prostate cancer, endometrial cancer, glioma, neurological cancer, skin cancer, head and neck cancer, stomach cancer, liver cancer, testis cancer, thyroid cancer, lymphoma, urothelial cancer, or myelodysplastic syndromes.

31. (canceled)

32. (canceled)

33. The method of claim 29, wherein the patient harbors tumor cell having an activating mutation in Fms-like tyrosine kinase 3 (FLT3), optionally wherein the activating mutation is an FLT3-ITD mutation.

34. (canceled)

35. (canceled)

36. (canceled)

37. A method of preparing CAR T or NK cells comprising: providing a population of autologous or allogeneic human T or NK cells and transducing the T or NK cells by a vector comprising the nucleic acid molecule of claim 1.

38. A method of preparing a CAR expressing cell comprising transducing the cell with the nucleic acid molecule of claim 1.


SEQUENCE LISTING (30 sequences)

SEQ ID NO: 1 - DNA, 1380 bp, Artificial Sequence
  source: /note=“Description of Artificial Sequence Synthetic polynucleotide”
atggggtggtcaagcattattctgtttctggtcgctaccgctacaggcgtccatcaggtccagctgcagcagcccggagccgaactggtgaagcccggcgcctccctgaagctgtcttgcaagagcagcggctacacattcacctcctattggatgcactgggtgcggcagcggcccggccacggcctggagtggatcggcgagatcgacccctctgatagctacaaggactataaccagaagtttaaggataaggccacactgaccgtggaccggtctagcaatacagcctacatgcacctgtcctctctgacctccgacgattctgccgtgtactattgcgccagggccatcaccacaacccctttcgatttttggggccagggcacaaccctgaccgtgagcagcggaggaggaggcagcggaggaggaggctccggcggcggcggctctgacatcgtgctgacccagtccccagccacactgagcgtgacccctggcgactccgtgtctctgagctgtcgggcctcccagtctatcagcaacaatctgcactggtatcagcagaagagccacgagtcccctaggctgctgatcaagtatgcctcccaatctatcagcggcatcccaagccgcttctccggctctggcagcggcacagacttcaccctgtctatcaacagcgtggagacagaggacttcggcgtgtatttttgtcagcagtctaatacatggccatatacattcggaggaggaactaaactggaaatcaaacgactcgagcccaaatcttgtgacaaaactcacacatgcccaccgtgcccggatcccaaaggtaccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 2 - protein, 460 aa, Artificial Sequence
  source: /note=“Description of Artificial Sequence Synthetic polypeptide”
MGWSSIILFLVATATGVHQVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKRLEPKSCDKTHTCPPCPDPKGTFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 18 aa, Artificial Sequence
  source: /note=“Description of Artificial Sequence Synthetic peptide”
MGWSSIILFLVATATGVH

SEQ ID NO: 4 - protein, 241 aa, Artificial Sequence
  source: /note=“Description of Artificial Sequence Synthetic polypeptide”
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSGGGGSGGGGSGGGGSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 5 - protein, 21 aa, Artificial Sequence
  source: /note=“Description of Artificial Sequence Synthetic peptide”
LEPKSCDKTHTCPPCPDPKGT

SEQ ID NO: 6 - protein, 27 aa, Artificial Sequence
  source: /note=“Description of Artificial Sequence Synthetic peptide”
FWVLVVVGGVLACYSLLVTVAFIIFWV

SEQ ID NO: 7 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 8 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 24 aa, Artificial Sequence
  source: /note=“Description of Artificial Sequence Synthetic peptide”
LEGGGEGRGSLLTCGDVEENPGPR

SEQ ID NO: 10 - protein, 354 aa, Artificial Sequence
  source: /note=“Description of Artificial Sequence Synthetic polypeptide”
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 11 - protein, 216 aa, Artificial Sequence
  source: /note=“Description of Artificial Sequence Synthetic polypeptide”
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTH

SEQ ID NO: 12 - DNA, 1380 bp, Artificial Sequence; identical to SEQ ID NO: 1
  source: /note=“Description of Artificial Sequence Synthetic polynucleotide”
  variation (6)..(6): /replace=“any other nucleotide”
  variation (10)..(15): /replace=“any other nucleotide”
  variation (18)..(18): /replace=“any other nucleotide”
  variation (21)..(21): /replace=“any other nucleotide”
  variation (24)..(24): /replace=“any other nucleotide”
  variation (27)..(28): /replace=“any other nucleotide”
  variation (33)..(33): /replace=“any other nucleotide”
  variation (36)..(36): /replace=“any other nucleotide”
  variation (39)..(39): /replace=“any other nucleotide”
  variation (48)..(48): /replace=“any other nucleotide”
  variation (54)..(54): /replace=“any other nucleotide”
  variation (63)..(63): /replace=“any other nucleotide”
  variation (75)..(75): /replace=“any other nucleotide”
  variation (78)..(78): /replace=“any other nucleotide”
  variation (81)..(81): /replace=“any other nucleotide”
  variation (84)..(84): /replace=“any other nucleotide”
  variation (87)..(87): /replace=“any other nucleotide”
  variation (96)..(96): /replace=“any other nucleotide”
  variation (99)..(99): /replace=“any other nucleotide”
  variation (102)..(102): /replace=“any other nucleotide”
  variation (105)..(106): /replace=“any other nucleotide”
  variation (117)..(117): /replace=“any other nucleotide”
  variation (124)..(128): /replace=“any other nucleotide”
  variation (132)..(132): /replace=“any other nucleotide”
  variation (138)..(138): /replace=“any other nucleotide”
  variation (145)..(146): /replace=“any other nucleotide”
  variation (150)..(150): /replace=“any other nucleotide”
  variation (166)..(166): /replace=“any other nucleotide”
  variation (172)..(172): /replace=“any other nucleotide”
  variation (177)..(177): /replace=“any other nucleotide”
  variation (180)..(180): /replace=“any other nucleotide”
  variation (183)..(183): /replace=“any other nucleotide”
  variation (189)..(189): /replace=“any other nucleotide”
  variation (201)..(201): /replace=“any other nucleotide”
  variation (207)..(207): /replace=“any other nucleotide”
  variation (210)..(210): /replace=“any other nucleotide”
  variation (213)..(213): /replace=“any other nucleotide”
  variation (219)..(219): /replace=“any other nucleotide”
  variation (222)..(222): /replace=“any other nucleotide”
  variation (225)..(225): /replace=“any other nucleotide”
  variation (228)..(228): /replace=“any other nucleotide”
  variation (234)..(234): /replace=“any other nucleotide”
  variation (237)..(237): /replace=“any other nucleotide”
  variation (246)..(246): /replace=“any other nucleotide”
  variation (252)..(252): /replace=“any other nucleotide”
  variation (262)..(262): /replace=“any other nucleotide”
  variation (267)..(267): /replace=“any other nucleotide”
  variation (274)..(274): /replace=“any other nucleotide”
  variation (276)..(276): /replace=“any other nucleotide”
  variation (279)..(281): /replace=“any other nucleotide”
  variation (285)..(285): /replace=“any other nucleotide”
  variation (303)..(305): /replace=“any other nucleotide”
  variation (307)..(309): /replace=“any other nucleotide”
  variation (315)..(315): /replace=“any other nucleotide”
  variation (318)..(318): /replace=“any other nucleotide”
  variation (321)..(321): /replace=“any other nucleotide”
  variation (324)..(324): /replace=“any other nucleotide”
  variation (330)..(330): /replace=“any other nucleotide”
  variation (333)..(333): /replace=“any other nucleotide”
  variation (336)..(336): /replace=“any other nucleotide”
  variation (342)..(342): /replace=“any other nucleotide”
  variation (345)..(345): /replace=“any other nucleotide”
  variation (348)..(348): /replace=“any other nucleotide”
  variation (351)..(351): /replace=“any other nucleotide”
  variation (354)..(354): /replace=“any other nucleotide”
  variation (357)..(357): /replace=“any other nucleotide”
  variation (360)..(360): /replace=“any other nucleotide”
  variation (366)..(366): /replace=“any other nucleotide”
  variation (369)..(369): /replace=“any other nucleotide”
  variation (372)..(372): /replace=“any other nucleotide”
  variation (375)..(375): /replace=“any other nucleotide”
  variation (384)..(384): /replace=“any other nucleotide”
  variation (390)..(390): /replace=“any other nucleotide”
  variation (393)..(393): /replace=“any other nucleotide”
  variation (396)..(396): /replace=“any other nucleotide”
  variation (399)..(399): /replace=“any other nucleotide”
  variation (402)..(404): /replace=“any other nucleotide”
  variation (406)..(408): /replace=“any other nucleotide”
  variation (411)..(411): /replace=“any other nucleotide”
  variation (414)..(414): /replace=“any other nucleotide”
  variation (417)..(417): /replace=“any other nucleotide”
  variation (420)..(423): /replace=“any other nucleotide”
  variation (426)..(426): /replace=“any other nucleotide”
  variation (429)..(429): /replace=“any other nucleotide”
  variation (432)..(432): /replace=“any other nucleotide”
  variation (444)..(444): /replace=“any other nucleotide”
  variation (450)..(450): /replace=“any other nucleotide”
  variation (456)..(456): /replace=“any other nucleotide”
  variation (459)..(459): /replace=“any other nucleotide”
  variation (465)..(465): /replace=“any other nucleotide”
  variation (468)..(468): /replace=“any other nucleotide”
  variation (474)..(474): /replace=“any other nucleotide”
  variation (483)..(483): /replace=“any other nucleotide”
  variation (487)..(489): /replace=“any other nucleotide”
  variation (495)..(495): /replace=“any other nucleotide”
  variation (498)..(498): /replace=“any other nucleotide”
  variation (501)..(501): /replace=“any other nucleotide”
  variation (504)..(506): /replace=“any other nucleotide”
  variation (510)..(512): /replace=“any other nucleotide”
  variation (516)..(518): /replace=“any other nucleotide”
  variation (522)..(523): /replace=“any other nucleotide”
  variation (529)..(530): /replace=“any other nucleotide”
  variation (535)..(536): /replace=“any other nucleotide”
  variation (540)..(540): /replace=“any other nucleotide”
  variation (549)..(549): /replace=“any other nucleotide”
  variation (552)..(552): /replace=“any other nucleotide”
  variation (564)..(564): /replace=“any other nucleotide”
  variation (567)..(567): /replace=“any other nucleotide”
  variation (570)..(573): /replace=“any other nucleotide”
  variation (576)..(576): /replace=“any other nucleotide”
  variation (582)..(582): /replace=“any other nucleotide”
  variation (585)..(585): /replace=“any other nucleotide”
  variation (591)..(591): /replace=“any other nucleotide”
  variation (594)..(594): /replace=“any other nucleotide”
  variation (606)..(606): /replace=“any other nucleotide”
  variation (612)..(612): /replace=“any other nucleotide”
  variation (615)..(615): /replace=“any other nucleotide”
  variation (619)..(621): /replace=“any other nucleotide”
  variation (624)..(624): /replace=“any other nucleotide”
  variation (630)..(632): /replace=“any other nucleotide”
  variation (634)..(634): /replace=“any other nucleotide”
  variation (636)..(636): /replace=“any other nucleotide”
  variation (640)..(642): /replace=“any other nucleotide”
  variation (646)..(647): /replace=“any other nucleotide”
  variation (651)..(654): /replace=“any other nucleotide”
  variation (657)..(657): /replace=“any other nucleotide”
  variation (663)..(663): /replace=“any other nucleotide”
  variation (669)..(669): /replace=“any other nucleotide”
  variation (672)..(674): /replace=“any other nucleotide”
  variation (684)..(684): /replace=“any other nucleotide”
  variation (693)..(693): /replace=“any other nucleotide”
  variation (696)..(696): /replace=“any other nucleotide”
  variation (699)..(699): /replace=“any other nucleotide”
  variation (702)..(702): /replace=“any other nucleotide”
  variation (705)..(705): /replace=“any other nucleotide”
  variation (714)..(714): /replace=“any other nucleotide”
  variation (720)..(720): /replace=“any other nucleotide”
  variation (724)..(725): /replace=“any other nucleotide”
  variation (729)..(729): /replace=“any other nucleotide”
  variation (732)..(732): /replace=“any other nucleotide”
  variation (738)..(738): /replace=“any other nucleotide”
  variation (741)..(741): /replace=“any other nucleotide”
  variation (744)..(744): /replace=“any other nucleotide”
  variation (753)..(753): /replace=“any other nucleotide”
  variation (756)..(756): /replace=“any other nucleotide”
  variation (759)..(759): /replace=“any other nucleotide”
  variation (762)..(762): /replace=“any other nucleotide”
  variation (771)..(771): /replace=“any other nucleotide”
  variation (777)..(1380): /replace=“any other nucleotide”
  misc_feature (1)..(1380): /note=“Variant positions given in the sequence have no preference with respect to those in the annotations for variant positions”

SEQ ID NO: 13 - protein, 323 aa, Artificial Sequence
  source: /note=“Description of Artificial Sequence Synthetic polypeptide”
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 14 - DNA, 777 bp, Artificial Sequence
  source: /note=“Description of Artificial Sequence Synthetic polynucleotide”
atgggatggagctctatcatcctcttcttggtagcaacagctacaggtgtccaccaggtccaactgcagcagcctggggctgagcttgtgaagcctggggcttcattgaagctgtcctgcaagtcttccgggtacaccttcaccagctactggatgcactgggtgaggcagaggcctggacatggccttgagtggatcggagagattgatccttctgacagttataaagactacaatcagaagttcaaggacaaggccacattgactgtggacagatcctccaacacagcctacatgcacctcagcagcctgacatctgatgactctgcggtctattattgtgcaagagcgattacgacgaccccctttgacttctggggccaaggcaccactctcacagtctcctcaggcggtggcggttctggtggcggtggctccggcggtggcggttctgatattgtgctaactcagtctccagccaccctgtctgtgactccaggagatagcgtcagtctttcctgcagggccagccagagtattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggagtgtatttctgtcaacagagtaacacctggccgtacacgttcggaggggggaccaagctggaaataaaacgg

SEQ ID NO: 15 - protein, 11 aa, Artificial Sequence
  source: /note=“Description of Artificial Sequence Synthetic peptide”
RASQSISNNLH

SEQ ID NO: 16 - protein, 7 aa, Artificial Sequence
  source: /note=“Description of Artificial Sequence Synthetic peptide”
YASQSIS

SEQ ID NO: 17 - protein, 9 aa, Artificial Sequence
  source: /note=“Description of Artificial Sequence Synthetic peptide”
QQSNTWPYT

SEQ ID NO: 18 - protein, 5 aa, Artificial Sequence
  source: /note=“Description of Artificial Sequence Synthetic peptide”
SYWMH

SEQ ID NO: 19 - protein, 17 aa, Artificial Sequence
  source: /note=“Description of Artificial Sequence Synthetic peptide”
EIDPSDSYKDYNQKFKD

SEQ ID NO: 20 - protein, 9 aa, Artificial Sequence
  source: /note=“Description of Artificial Sequence Synthetic peptide”
AITTTPFDF

SEQ ID NO: 21 - protein, 118 aa, Artificial Sequence
  source: /note=“Description of Artificial Sequence Synthetic polypeptide”
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 22 - protein, 108 aa, Artificial Sequence
  source: /note=“Description of Artificial Sequence Synthetic polypeptide”
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 23 - DNA, 546 bp, Artificial Sequence
  source: /note=“Description of Artificial Sequence Synthetic polynucleotide”
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 24 - DNA, 454 bp, Artificial Sequence
  source: /note=“Description of Artificial Sequence Synthetic polynucleotide”
ttaattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaag

SEQ ID NO: 25 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 26 - protein, 952 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 27 - protein, 5 aa, Artificial Sequence
  source: /note=“Description of Artificial Sequence Synthetic peptide”
GGGGS

SEQ ID NO: 28 - DNA, 648 bp, Artificial Sequence
  source: /note=“Description of Artificial Sequence Synthetic polynucleotide”
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccat

SEQ ID NO: 29 - protein, 8 aa, Artificial Sequence
  source: /note=“Description of Artificial Sequence Synthetic peptide”
  VARIANT (2)..(2): /replace=“Ile”
  MOD_RES (4)..(4): Any amino acid
  SITE (1)..(8): /note=“Variant residues given in the sequence have no preference with respect to those in the annotations for variant positions”
  source: /note=“See specification as filed for detailed description of substitutions and preferred embodiments”
DVEXNPGP

SEQ ID NO: 30 - DNA, 599 bp, Artificial Sequence
  source: /note=“Description of Artificial Sequence Synthetic polynucleotide”
atcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaatt